(19) United States
(12) Patent Application Publication
(10) Pub. No.: US 2016/0340659 A1
(43) Pub. Date: Nov. 24, 2016

(54) ACTIN BINDING PEPTIDES AND COMPOSITIONS COMPRISING SAME FOR INHIBITING ANGIOGENESIS AND TREATING MEDICAL CONDITIONS ASSOCIATED WITH SAME

(71) Applicant: YISSUM RESEARCH AND DEVELOPMENT COMPANY OF THE HEBREW UNIVERSITY OF JERUSALEM LTD., Jerusalem (IL)

(72) Inventors: Oded SHOSEYOV, Karme Yosef (IL); Betty SCHWARTZ, Rechovot (IL); Shani DORON, Rechovot (IL); Liron NESIEL, Rechovot (IL); Assaf FRIEDLER, Mevasseret Zion (IL); Hadar AMARTELY, MaAle Adumim (IL); Levava ROIZ, Kiryat-Ono (IL); Patricia SMIRNOFF, Rehovot (IL); Iris LEWIN, Mevaseret Zion (IL)

(21) Appl. No.: 15/113,187

(22) PCT Filed: Jan. 29, 2015
(86) PCT No.: PCT/IL2015/050108

§ 371 Date: Jul. 21, 2016

Related U.S. Application Data
(60) Provisional application No. 61/933,363, filed on Jan. 30, 2014.

(51) Int. Cl. C12N 9/22

(52) U.S. Cl. CPC C12N 9/22; C12Y 301/27; A61K 38/00

(57) ABSTRACT

The present invention, in some embodiments thereof, relates to biologically active peptides and, more particularly, but not exclusively, to peptides from T2 RNase (RNASET2) having actin binding, pharmaceutical compositions comprising the same, therapeutic use thereof and methods for their production.

### FIELD AND BACKGROUND OF THE INVENTION

The present invention, in some embodiments thereof, relates to biologically peptides and, more particularly, but not exclusively, to peptides from T2 RNase having actin binding, pharmaceutical compositions comprising the same, therapeutic use thereof and methods for their production.

RNases of the T2 RNase family are single strand-specific endoribonucleases acting through base-non-specific nucleolytic cleavage, are broadly distributed throughout all eukaryotic, plant and animal and also bacterial and viral species and are typically characterized by a strongly acidic pH optimum for RNase activity. The T2 RNases have demonstrated a wide range of biological roles, including “housekeeping” functions, prevention of “self” pollination in plants, antimicrobial defense and tumor suppression in humans.

Human RNASET2 is a T₂-RNase glycoprotein encoded by the RNASET2 gene which is located on chromosome 6 (6q27) and known as a tumor suppressor gene (Trubia et al. 1997. Genomics 42:342-344; Acquati et al. 2001. Meth Mol Biol 160:87-101). Mutation and loss of function of RNASET2 has been associated with increased tumorigenicity and cancer, including carcinomas of the ovary, breast, uterus, stomach, liver, colon/rectum, kidney and hematologic malignancies, such as non-Hodgkin, B-cell lymphoma and acute lymphoblastic leukemia.

Acquati et al. (PNAS 2011, PNAS 2013) have shown that overexpression of human RNASET2 can suppress growth of ovarian and other cancerous tumors in in-vivo experiments, independently of its ribonucleolytic activity, although no such anti-tumorigenic activity was observed in the transformed cells in-vitro (Acquati et al, 2011 and Liu et al, 2002).

In PCT WO 2001/15531 (incorporated herein by reference in its entirety), the present inventors have shown that recombinant B1 T2 RNase from A. niger can inhibit in-vitro tumor growth, tumorigenesis metastatic transformation and metastatic spread in-vivo, and effectively bind actin in both cell-free systems and on the surface of cancer cells in culture. Further investigation revealed in-vitro inhibition by A. niger RNase B1 of angiogenesis, inhibition of cell extension and cell migration in cancer cells and endothelial cells, and in-vivo inhibition of tumor angiogenesis metastatic transformation and metastatic spread, in a variety of cancer cell lines, and actin binding activity on Western blots (see U.S. Pat. Nos. 7,811,981 and 8,236,543, incorporated herein by reference in their entirety). Quantitation of actin binding of the A. niger RNase B1 indicated high affinity binding of both catalytically active and inactive RNase to actin.

PCT WO2006/035439 (incorporated herein by reference in its entirety) to Roiz et al discloses the cloning and expression, in P. pastoris, of human RNASET2, having tumor suppressing and anti-angiogenic activity in-vivo and in-vitro, as well as having strong actin-binding properties. None of these properties required the ribonucleolytic activity of the protein.

PCT WO2010/04993 to Shoseyov et al, and Nesiel-nuttman et al (Oncotarget 2014 22:11464-78) (both incorporated herein by reference in their entirety) disclose the cloning and efficient expression in bacteria of truncated human RNASET2 protein, lacking part of the RNase catalytic domain, but retaining full tumor suppressing, anti-angiogenic and actin-binding properties.

Conserved domains and structural motifs of T2 RNase enzymes were investigated by crystallography (MacIntosh, 2011), revealing a general consensus structure comprising 7 α-helices and 8 β-strands, despite the low inter-species sequence homology. A complete structural analysis of human RNASET2, concurring the structural consensus of the T2 family, has been elucidated (Thorn et al, 2012).

Kumar et al, (J Mol Model, 2013; 19:613-21) and Gundampati et al (J Mol Model 2012, 18:653-62) using computer-generated, three dimensional models of A. niger RNase B1 and actin in in-silico molecular dynamics docking simulations, postulated an RNase B1 actin-binding sequence, and proposed that A. niger RNase B1 interaction with cell surface actin in neoplastic cells could provide a rationale for design of anti-cancer drugs based on RNase B1. De Leeuw et al (2012), on the basis of three dimensional conformation and amino acid sequence homology, proposed two actin binding sites for RNase B1, remote from those postulated by Kumar et al.

Additional background art includes Nesiel-nuttman et al, Oncoscience 2014, Advanced Publication Nov. 26, 2014), Medinger et al (J Angiogenesis Res 2010; 2:10) and Rosca et al, (Curr. Res Biotech, 2011; 12:1101).

### SUMMARY OF THE INVENTION

According to an aspect of some embodiments of the present invention there is provided an isolated peptide comprising a core amino acid sequence which comprises at least 10 amino acids of helix 5 of human RNASET2, or naturally occurring homologues thereof, or conservative substitutions thereof, wherein the peptide is 23-50 amino acids in length and wherein the peptide binds actin.

According to an aspect of some embodiments of the present invention there is provided a composition of matter comprising the isolated peptide of the invention formulated with a cell penetrating agent.

According to an aspect of some embodiments of the present invention there is provided a composition of matter comprising the isolated peptide of the invention formulated with a targeting moiety.

According to some embodiments of the invention the cell penetrating agent comprises a cell penetrating peptide covalently attached to the peptide.

According to an aspect of some embodiments of the present invention there is provided a nucleic acid construct comprising a polynucleotide encoding the peptide of the invention.

According to some embodiments of the invention the nucleic acid construct comprises a promoter element functional in mammalian cells.

According to an aspect of some embodiments of the present invention there is provided a pharmaceutical composition comprising the isolated peptide of the invention or the composition of the invention and a pharmaceutically acceptable carrier.

According to an aspect of some embodiments of the present invention there is provided an article of manufacture comprising the isolated peptide of the invention or the composition of the invention and packaging material.

According to an aspect of some embodiments of the present invention there is provided a method of preventing, inhibiting and/or reversing colonization, differentiation and/or development of abnormally proliferating cells in a subject, comprising administering a therapeutically effective amount of the isolated peptide of the invention, the composition of the invention or the pharmaceutical composition of the invention to the subject.

According to an aspect of some embodiments of the present invention there is provided a method of treating or preventing a proliferative disorder or disease in a subject, comprising administering a therapeutically effective amount of the isolated peptide of the invention, the composition of the invention or the pharmaceutical composition of the invention to the subject.

According to some embodiments of the invention the proliferative disorder or disease is selected from the group consisting of papilloma, blastoglioma, Kaposi's sarcoma, melanoma, lung cancer, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's disease, Burkitt's disease, arthritis, rheumatoid arthritis, diabetic retinopathy, pathogenic angiogenesis, restenosis, in-stent restenosis and vascular graft restenosis, proliferative vitreoretinopathy, chronic inflammatory proliferative disease, dermatofibroma and psoriasis.

According to an aspect of some embodiments of the present invention there is provided a method of inhibiting angiogenesis in a subject, the method comprising administering to the subject a therapeutically effective amount of the isolated peptide of the invention, the composition of the invention or the pharmaceutical composition of the invention to the subject.

According to an aspect of some embodiments of the present invention there is provided a method of inhibiting metastatic transformation and/or spread of a tumor in a subject, the method comprising administering to the subject a therapeutically effective amount of the isolated peptide of the invention, the composition of the invention or the pharmaceutical composition of the invention to the subject.

According to some embodiments of the invention the angiogenesis is tumor angiogenesis.

According to some embodiments of the invention the administering is effected by an administration mode selected from the group consisting of oral administration, intravenous administration, subcutaneous administration, systemic administration, topical administration, transmucosal administration, parenteral administration, rectal administration and inhalation.

According to an aspect of some embodiments of the present invention there is provided a method of inhibiting angiogenesis in a tissue, the method comprising contacting the tissue with a therapeutically effective amount of the isolated peptide of the invention, the composition of the invention or the pharmaceutical composition of the invention.

According to some embodiments of the invention the at least 10 amino acids of helix 5 of human RNASET2 correspond to positions 108-121 of SEQ ID NO.: 1.

According to some embodiments of the invention the amino acids of the core amino acid sequence corresponding to positions 116 and 122 of SEQ ID 1 are negatively charged amino acids.

According to some embodiments of the invention the amino acid of the core amino acid sequence corresponding to position 119 is a positively charged amino acid.

According to some embodiments of the invention the isolated peptide comprises at least one additional amino acid sequence.

According to some embodiments of the invention the at least one additional amino acid sequence comprises: a human RNASET2 sequence or conservative amino acid substitutions thereof, or a homologous T2RNase sequence or conservative amino acid substitutions thereof.

According to some embodiments of the invention the at least one additional amino acid sequence is of a non-human T2 RNase sequence or conservative amino acid substitutions thereof.

According to some embodiments of the invention the at least one additional amino acid sequence is of the same species as of the core sequence.

According to some embodiments of the invention the at least one additional amino acid sequence is heterologous to the core sequence.

According to some embodiments of the invention the at least one additional amino acid sequence is an amino acid sequence selected from the group consisting of SEQ ID NO: 25 (1-19 of SEQ ID NO: 1), SEQ ID NO: 26 (20-24 of SEQ ID NO: 1), SEQ ID NO:27 (25-50 of SEQ ID NO: 1), SEQ ID NO: 28 (51-65 of SEQ ID NO: 1), SEQ ID NO: 29 (66-72 of SEQ ID NO: 1), SEQ ID NO: 30 (72-83 of SEQ ID NO: 1), SEQ ID NO: 31 (84-93 of SEQ ID NO: 1), SEQ ID NO: 32 (94-107 of SEQ ID NO: 1), SEQ ID NO: 33 (123-129 of SEQ ID NO: 1), SEQ ID NO: 34 (134-160 of SEQ ID NO: 1), SEQ ID NO: 35 (161-190 of SEQ ID NO: 1) and SEQ ID NO: 36 (191-231 of SEQ ID NO: 1) or a portion thereof.

According to some embodiments of the invention the at least one additional amino acid sequence is positioned N terminally to the core amino acid sequence.

According to some embodiments of the invention the at least one additional amino acid sequence is positioned C terminally to the core amino acid sequence.

According to some embodiments of the invention the at least one additional amino acid sequence is a helix.

According to some embodiments of the invention the helix is selected from the group consisting of SEQ ID NOs. 37, 38 and 39-56 of human T2RNASE.

According to some embodiments of the invention the at least one additional amino acid sequence comprises at least two additional amino acid sequences, flanking the core amino acid sequence.

According to some embodiments of the invention the at least one additional amino acid sequence is linked via a linker to the core amino acid sequence.

According to some embodiments of the invention the core amino acid sequence is selected from the group consisting of SEQ ID NO: 2-24.

According to some embodiments of the invention the core amino acid sequence comprises SEQ ID NO: 127.

According to some embodiments of the invention the core amino acid sequence is as set forth in SEQ ID NO:81.

According to some embodiments of the invention the isolated peptide is 23 amino acids in length.

According to some embodiments of the invention the core amino acid sequence is as set forth in SEQ ID NO: 82.

According to some embodiments of the invention the core amino acid sequence is as set forth in SEQ ID NO:83.

According to some embodiments of the invention the core amino acid sequence is as set forth in SEQ ID NO: 57.

According to some embodiments of the invention the isolated peptide is 24 amino acids in length.

According to some embodiments of the invention the isolated peptide comprises the amino acid sequence:

X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃;

wherein X₁ and X₈ are selected from group E; X₂, X₄, X₁₅ and X₂₁ are selected from group A; X₃, X₉, X₁₉ and X₂₃ are selected from group C; X₅, X₇, X₁₁, X₁₃, X₁₆, X₁₇, X₁₈; X₂₀ and X₂₂ are selected from group D and X₆, X₁₀, X₁₂ and X₁₄ are selected from group B;

wherein group A consists of small, aliphatic, non-polar or slightly polar amino acid residues, group B consists of polar, negatively charged amino acid residues and their (uncharged) amides; group C consists of polar, positively charged amino acid residues, group D consists of large, aliphatic non-polar amino acid residues and group E consists of aromatic residues.

According to some embodiments of the invention, group A comprises an amino acid sequence selected from the group consisting of amino acids Ala, Ser, Thr, Pro and Gly; group B consists of amino acids Asp, Asn, Glu and Gln; group C consists of amino acids His, Arg and Lys; group D consists of amino acids Met, Leu, Ile, Val and Cys and group E consists of amino acids Phe, Tyr and Trp.

According to some embodiments of the invention the isolated peptide further comprises an additional N-terminal amino acid sequence selected from the group consisting of X_{0″}X_{0′}X₀, X_{0′}X₀ and X₀ positioned N-terminally to X₁, wherein X_{0′} and X_{0″} are selected from group C and X₀ is selected from group E.

According to some embodiments of the invention the isolated peptide comprises SEQ ID NO: 83.

According to some embodiments of the invention the isolated peptide, comprises SEQ ID NO: 82.

According to some embodiments of the invention the isolated peptide comprises SEQ ID NO: 81.

According to some embodiments of the invention the isolated peptide comprises SEQ ID NO: 57.

According to some embodiments of the invention the isolated peptide of any one of claims 1-27, comprises SEQ ID NO: 130.

According to some embodiments of the invention the isolated peptide comprises SEQ ID NO: 133.

According to an aspect of some embodiments of the present invention there is provided an isolated peptide comprising a core amino acid sequence, which comprises at least 5 amino acids of helix 5 of human RNASET2, or naturally occurring homologues thereof, or conservative substitutions thereof, wherein the peptide is 5-22 amino acids in length and wherein the peptide binds actin.

According to some embodiments of the invention the isolated peptide is selected from the group consisting of SEQ ID NO: 62, 131 and 132.

According to some embodiments of the invention the isolated peptide comprises at least one synthetic amino acid.

According to some embodiments of the invention the isolated peptide has a biological activity other than actin binding.

According to some embodiments of the invention the biological activity is inhibition of angiogenesis.

According to some embodiments of the invention the angiogenesis is tumor angiogenesis.

According to some embodiments of the invention the biological activity is preventing, inhibiting and/or reversing colonization, differentiation and/or development of abnormally proliferating cells, cell motility and metastatic transformation.

According to some embodiments of the invention the abnormally proliferating cells are cancer cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a three dimensional representations of human T2RNASE protein, from Thorn et al (2012), showing the 8 β-strands and the 7 α-helices;

FIG. 2 is an alignment of T2 RNase peptides, indicating their positions relative to human RNASET2 protein coordinates. Charged amino acids are shaded in grey. Actin binding efficacy, by ELISA, of the individual peptides is indicated in the “ELISA” column. “−−” indicates poor or no actin binding, “−+” indicates weak-moderate actin binding and “++” indicates significant—robust actin binding;

FIG. 3 is a structural analysis of the human RNASET2 polypeptide and 26 amino acid T2 peptide fragment. In green—human RNASET2 structure. In blue—T2 peptide K108-K133 (SEQ ID NO: 57), including helixes 5 and 6. Structural analysis using the PyMole biomolecular visualization program;

FIG. 4 is a graph showing actin binding by purified truncated T2 polypeptides trT2-49 (SEQ ID NO: 84) and trT2-49m (SEQ ID NO.129), representing the trT2-49 polypeptide devoid of amino acids E120-V141, as measured in the solid-phase actin binding assay (immobilized actin) with increasing concentrations (0-500 ng/100 μl well) of the truncated T2 protein. Diamonds represent trT2-49; squares represent trT2-49m polypeptides. Note the effective and concentration-dependent binding of actin by both truncated polypeptides in the range of 0-100 ng/100 μl well;

FIGS. 5A and 5B are graphs showing actin binding by purified T2 peptides A103-Q159 (22A—SEQ ID NO: 60) and K108-K133 (22B—SEQ ID NO: 57), compared to the truncated T2 polypeptide trT2-49 (SEQ ID NO: 84) as measured in the solid-phase actin binding assay (immobilized actin) with increasing concentrations (0-500 ng/100 μl well) of the truncated T2 protein. FIG. 5A-Diamonds represent trT2-49; Xs represent peptides of SEQ ID NO: 60. FIG. 5B—Diamonds represent trT2-49; triangles represent peptides of SEQ ID NO: 57. Note the effective and concentration-dependent binding of actin by both T2 peptides in the range of 0-100 ng/100 μl well;

FIGS. 6A-6D illustrate in-vitro inhibition of angiogenesis in the HUVEC model by T2 peptides. HUVEC cells plated on Matrigel™ were exposed to 2 μM of T2 peptides A103-Q159 (SEQ ID NO: 60), K108-K133 (SEQ ID NO: 57) or K108-L123 (SEQ ID NO: 67), in addition to 1 μg/ml of angiogenin or VEGF as the angiogenic agent, as indicated. Control wells received PBS. FIG. 6B-Results were assessed by imaging software, and quantified, and expressed relative to tube formation with angiogenin or VEGF alone (100%) Column 1=angiogenin, column 2=angiogenin+SEQ ID NO: 60, column 3=angiogenin+SEQ ID NO: 57, column 4=angiogenin+SEQ ID NO: 60, Column 5=VEGF, column 6=VEGF+SEQ ID NO: 60, column 7=VEGF+SEQ ID NO: 57, column 8=VEGF+SEQ ID NO: 60. FIGS. 6C and 6D illustrate the inhibitory effect of truncated human T2 polypeptide trT2-49m (SEQ ID NO: 129) on angiogenin and VEGF induced HUVEC tube formation. Note the significant inhibition of HUVEC tube formation by all of the T2 peptides assayed, and in particular, K108-K133 (SEQ ID NO: 57);

FIGS. 7A-7D are fluorescent micrographs showing the localization of a green fluorescent labeled T2 peptide into HUVEC cells 10 minutes after treatment of the cells with the peptide. T2 peptide (K108-K133, SEQ ID NO: 57) is labeled green, visible in the perinuclear cytoplasm after 10 minutes (FIG. 7A), 2 hours (FIG. 7B) and 8 hours (FIG. 7C). At 24 hours (FIG. 7D) a greatly diminished fluorescence was detected;

FIGS. 8A1-8C4 are fluorescent micrographs showing the co-localization of the green fluorescent-tagged human RNASET2 peptide and angiogenin (using red fluorescent tagged anti-angiogenin) in HUVEC cells after treatment of the cells with the peptide and angiogenin. (FIG. 8A): human T2 peptide K108-K133 (SEQ ID NO: 57) (green). (FIG. 8B): anti-angiogenin (red). (FIG. 8C): The integration of 8A and 8B (nucleus in blue, peptide in green and angiogenin in red). (FIG. 8A1): 10 min of incubation with the peptide and angiogenin—most of the peptide was localized outside the cell (green). (FIGS. 8A2-8A3): 2 h of incubation with the peptide and angiogenin-some of the peptide was observed in the cytoplasm surrounding the nucleus and some was located outside the cell (green). (FIG. 8A4): 8 h of incubation with the peptide and angiogenin—some of the peptide was observed in the cytoplasm surrounding the nucleus and some was located outside the cell (green). (FIG. 8B1): anti-angiogenin, 10 min of incubation with the peptide and angiogenin-angiogenin was located in the nucleus, the cytoplasm surrounding the nucleus, on the actin fibers and outside the cell (red). Same angiogenin localization was observed after 2 and 8 h of incubation with the peptide and angiogenin (FIGS. 8B2, 8B3: 2 h. 8B4: 8 h) (red). (FIGS. 8C1-8C4): angiogenin and peptide K108-K133 (SEQ ID NO: 57) generally co-localized mainly outside the cell after 10 min (FIG. 8C1), 2 h (FIGS. 8C2, 8C3) and 8 h (FIG. 8C4);

FIGS. 9A-9D are graphs illustrating in-vivo anti-angiogenic effects of human RNASET2 peptide in the chick Chorio-Allantoic Membrane (CAM) angiogenesis assay. Following 3 days incubation, fertilized eggs were cracked into dishes and embryos allowed to developed a vascular network. On the eighth day of incubation 3 μg of human RNASET2, or RNASET2 peptides [FIG. 9A-human RNASET2, FIG. 9B-trT2-49, FIG. 9C-peptide A103-Q159 (SEQ ID NO: 60), FIG. 9D-peptide K108-K133 (SEQ ID NO: 57), black diamonds] or 5 μl PBS (control, grey squares) was applied onto paper disks placed on the developing vascular regions, the application repeated each day for four days. The number of blood vessels around the treated disk was counted every day. Note that both of the two peptides, as well as the full length hRNASET2 or truncated trT2-49 exhibit discernible anti-angiogenic activity compared to the controls.

FIG. 10 is a graphic representation of the anti-tumor effect of intravenous T2 RNase peptide administration in the mouse xenograft model. Super metastatic A375SM cells were injected to produce subcutaneous tumors in Hds-athymic nude mice. Tumor growth was monitored regularly and treatments with the T2 RNase peptide (peptide 108-133, SEQ ID NO: 57) initiated when approximately 100 m³ tumor volume was achieved. Treatment groups (10 mice per group) include: Control (PBS, 3× per week), T2 Peptide (2.5 mg/kg, 3× per week), T2 Peptide (2.5 mg/kg, 7× per week), T2 Peptide (5.0 mg/kg, 3× per week), T2 Peptide (7.5 mg/kg, 3× per week). At completion (40 days), tumors were excised, weight recorded and prepared for histopathological assessment. Note the strong anti-tumor effect of the T2 Peptide at 2.5 mg/Kg, 3× per week;

FIG. 11 is a graphic representation of the anti-tumor effect of intra-tumoral T2 RNase peptide administration in the mouse xenograft model. Tumor production and protocol as in FIG. 10. Treatment groups (10 mice per group) include: Control (PBS, 3× per week) and T2 Peptide (2.5 mg/kg, 3× per week). At completion (40 days), tumors were excised, weight recorded and prepared for histopathological assessment. Note the strong anti-tumor effect of intratumoral administration of the T2 Peptide at 2.5 mg/Kg, 3× per week;

FIG. 12 shows the histopathological analysis of an A375SM xenograft tumor section, stained with hematoxylin and eosin. Note the normally appearing cells of the peritumoral region and the apoptotic/necrotic cells of the intra-tumoral region;

FIGS. 13A-13D show both hematoxylin-eosin staining (FIGS. 13A and 13B) and apoptosis analysis (FragEL™ DNA Fragmentation Detection Kit) (FIGS. 13C and 13D) of tumor sections from mice receiving control treatment (intravenous PBS, 3× per week). Note the euchromatic and proliferating nuclei and sporadic apoptotic nuclei in the intratumoral region. FIGS. 13A and 13C—×100 magnification, FIGS. 13B and 13D-×200 magnification);

FIGS. 14A-14D show both hematoxylin-eosin staining (FIGS. 14A and 14B) and apoptosis analysis (FragEL™ DNA Fragmentation Detection Kit) (FIGS. 14C and 14D) of tumor sections from mice treated with the T2 Peptide, 2.5 mg/Kg (intravenous, 3× per week). Note the compact dense nuclei and abundant apoptotic (dark) nuclei in the intratumoral region. FIGS. 14A and 14C—×100 magnification, FIGS. 14B and 14D-×200 magnification);

FIGS. 15A-15D show both hematoxylin-eosin staining (FIGS. 15A and 15B) and apoptosis analysis (FragEL™ DNA Fragmentation Detection Kit) (FIGS. 15C and 15D) of tumor sections from mice treated with the T2 Peptide, 5.0 mg/Kg (intravenous, 3× per week). Note the compact dense nuclei and abundant apoptotic (dark) nuclei in the intratumoral region, similar to the anti-tumor effect of the 2.5 mg/Kg dose. FIGS. 15A and 15C—×100 magnification, FIGS. 15B and 15D—×200 magnification);

FIGS. 16A-16D show both hematoxylin-eosin staining (FIGS. 16A and 16B) and apoptosis analysis (FragEL™ DNA Fragmentation Detection Kit) (FIGS. 16C and 16D) of tumor sections from mice treated with the T2 Peptide, 7.5 mg/Kg (intravenous, 3× per week). Note the compact dense nuclei and abundant apoptotic (dark) nuclei in the intratumoral region, similar to the anti-tumor effect of the 2.5 and 5.0 mg/Kg dose. FIGS. 16A and 16C—×100 magnification, FIGS. 16B and 16D—×200 magnification);

FIGS. 17A-17D show both hematoxylin-eosin staining (FIGS. 17A and 17B) and apoptosis analysis (FragEL™ DNA Fragmentation Detection Kit) (FIGS. 17C and 17D) of tumor sections from mice treated daily with the T2 Peptide, 2.5 mg/Kg (intravenous, 7× per week). Although no effect on tumor volume was observed at this regimen, the histopathology and apoptosis analysis show clear antitumor effects similar to those of the 3× per week regimen. FIGS. 17A and 17C—×100 magnification, FIGS. 17B and 17D—×200 magnification);

FIGS. 18A-18D show both hematoxylin-eosin staining (FIGS. 18A and 18B) and apoptosis analysis (FragEL™ DNA Fragmentation Detection Kit) (FIGS. 18C and 18D) of tumor sections from mice receiving intratumoral control treatment (PBS, intratumorally, 3× per week). Note the disruption of tissue morphology, tumor tissue tearing and internal hemorrhaging (arrows in FIGS. 18A and 18B), and occasional apoptotic nuclei associated with damaged tissues (arrows in FIGS. 18C and 18D). FIGS. 18A and 18C—×100 magnification, FIGS. 18B and 18D—×200 magnification);

FIGS. 19A-19D show both hematoxylin-eosin staining (FIGS. 19A and 19B) and apoptosis analysis (FragEL™ DNA Fragmentation Detection Kit) (FIGS. 19C and 19D) of tumor sections from mice receiving intratumoral T2 Peptide treatment (2.5 mg/Kg T2 Peptide, intratumorally, 3× per week). Note the disruption of tissue morphology, tumor tissue tearing and internal hemorrhaging in the hematoxylin and eosin stained sections (FIGS. 19A and 19B), similar to the control intratumoral regimen. Note, however, the abundant apoptotic (dark staining) nuclei in the tumors of the intratumoral T2 peptide-treated mice. FIGS. 19A and 19C—×100 magnification, FIGS. 19B and 19D—×200 magnification);

FIGS. 20A-20B show vascular structures in hematoxylin-eosin-stained tumor sections from mice receiving control treatment (intravenous PBS, 3× per week). Note the dense masses of tumor cells, some touching the vascular structures (arrow in FIG. 20A) and intact vascular endothelial cells (arrow in FIG. 20B). FIG. 20A—×200 magnification, FIG. 20B—×400 magnification);

FIGS. 21A-21B show the tumor anti-angiogenic effects of treatment with the T2 Peptide. FIGS. 21A and 21B show vascular structures in hematoxylin-eosin-stained tumor sections from mice receiving intravenous T2 Peptide (2.5 mg/Kg, intravenous, 3× per week). Note the numerous necrotic and apoptotic cells among the cancer cells (dark arrows). The necrotic/apoptotic cells are seen to accumulate around the vasculature, while disruption of the endothelial structure is apparent (see FIG. 21B, light arrow). FIG. 21A—×200 magnification, FIG. 21B—×400 magnification);

FIGS. 22A-22B show the tumor anti-angiogenic effects of treatment with the T2 Peptide. FIGS. 22A and 22B show vascular structures in hematoxylin-eosin-stained tumor sections from mice receiving 5.0 mg/Kg intravenous T2 Peptide (3× per week). Note the viable cancer cells surrounding many of the vascular structures (FIG. 22A, arrow), and the partial disruption in endothelial cell structure (FIG. 22B, arrow). FIG. 22A—×200 magnification, FIG. 22B—×400 magnification);

FIGS. 23A-23B show the tumor anti-angiogenic effects of treatment with the T2 Peptide. FIGS. 23A and 23B show vascular structures in hematoxylin-eosin-stained tumor sections from mice receiving 7.5 mg/Kg intravenous T2 Peptide (3× per week). Histopathology and vascular structures appear similar to those of the control treatment. FIG. 23A—×200 magnification, FIG. 23B—×400 magnification);

FIGS. 24A-24B show the tumor anti-angiogenic effects of treatment with the T2 Peptide. FIGS. 24A and 24B show vascular structures in hematoxylin-eosin-stained tumor sections from mice receiving 2.5 mg/Kg daily intravenous T2 Peptide (7× per week). Despite the apparent lack of effect on tumor volume, daily administration of 2.5 mg/kg appeared to be effective in causing necrosis and apoptosis of cells surrounding the vasculature and disrupting endothelial structures. FIG. 24A—×200 magnification, FIG. 24B—×400 magnification);

FIGS. 25A-25B show vascular structures in hematoxylin-eosin-stained tumor sections from mice receiving intratumoral control treatment (intratumoral PBS, 3× per week). Disruption of tissue morphology, internal hemorrhaging and tumor tissue tearing is observed (see arrow in FIG. 25A), but vascular endothelium is undisturbed, with tumor cells touching the vascular structures (see arrow in FIG. 25B), similar to control intravenous administration. FIG. 25A—×200 magnification, FIG. 25B—×400 magnification);

FIGS. 26A-26B show vascular structures in hematoxylin-eosin-stained tumor sections from mice receiving 2.5 mg/Kg daily intratumoral T2 Peptide (3× per week). Characteristic disruption of tissue morphology, internal hemorrhaging and tumor tissue tearing is observed (see arrow in FIG. 26A). Viable tumor cells can be observed accumulating in the vicinity of vascular structures (see arrow in FIG. 26B). FIG. 26A—×200 magnification, FIG. 26B—×400 magnification).

### DESCRIPTION OF SPECIFIC EMBODIMENTS OF THE INVENTION

The present invention, in some embodiments thereof, relates to biologically active peptides and, more particularly, but not exclusively, to peptides from T2 RNase having actin binding and other biological activity, pharmaceutical compositions comprising the same, therapeutic use thereof and methods for their production.

Members of the T2 RNase family possess anti-clonogenic, anti-angiogenic, anti-tumorigenic and anti-metastatic activity of enormous therapeutic potential. This non-ribonucleolytic activity has been correlated with actin binding capacity common to T2 family members. The anti-tumorigenic, anti-clonogenic, anti-angiogenic and anti-metastatic properties of members of the T2 RNase family have been shown to be independent of the ribonucleolytic functions of the T2 proteins. Enzymatically inactivated RNase B1 retained all of the, anti-tumorigenic, anti-metastatic and anti-angiogenic properties of the catalytically active enzyme (see, for example, Roiz, 2006; Smirnoff, 2006; U.S. Pat. No. 7,811,981, incorporated herein by reference in its entirety). Yet further, truncation of recombinant human RNASET2, removing the entire 50 or 70 N-terminal amino acids comprising part of the ribonuclease catalytic center, did not impair the anti-clonogenic, anti-tumorigenic, anti-metastatic and anti-angiogenic properties nor the actin binding of the recombinant, truncated protein (PCT WO2010/04993 to Shoseyov et al, incorporated herein by reference in its entirety). However, the sequences or motifs responsible for T2 RNase actin binding and the anti-clonogenic, anti-angiogenic, anti-metastatic and anti-tumorigenic activity are not known, and are the subject of much speculation (see, for example Kumar et al, 2012 and 2013), with some groups suggesting critical sequences located in the N-terminal region (see, for example, Kumar et al, 2013 and Gundampati et al, 2012).

The present inventors, working with human RNASET2 proteins and protein fragments, have located, through laborious and intensive experimentation and screening, a critical actin binding domain within the amino acid sequence of the RNASET2 polypeptide, in a region which was previously dismissed by at least some researchers as devoid of actin biding activity. The present inventors have identified a plurality of peptides based on this core sequence, comprising peptides both homologous and heterologous to human RNASET2 sequences which are characterized by actin binding activity. Actin binding peptides having anti-angiogenic and anti-tumor activity have also been identified. Thus, the actin binding T2 RNase peptides of the invention can be used for a variety of therapeutic and research purposes.

Thus, according to an aspect of the present invention, there is provided an isolated peptide comprising a core amino acid sequence, the core sequence comprising at least 10 amino acids of helix 5 of human T2 RNase, or naturally occurring homologues thereof, or conservative substitutions thereof, wherein the peptide is 23-50 amino acids in length and wherein the peptide binds actin.

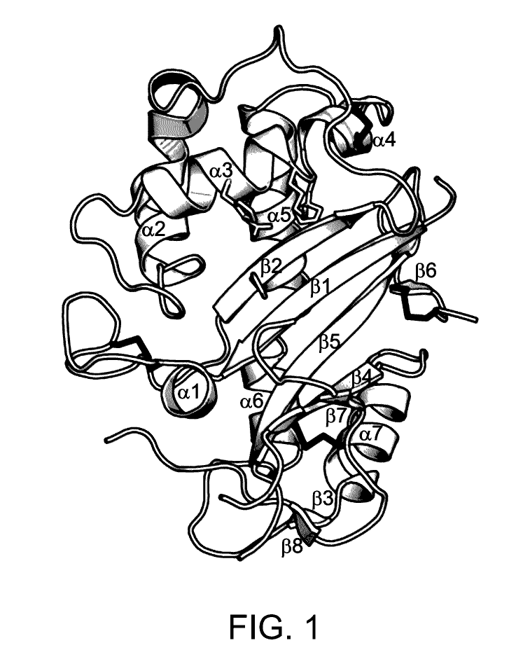

As used herein, the term “human RNASET2” relates to the human member of the T2 family of RNases, previously known as “RNase6Pl” or “human T2 RNase”. “RNASET2” (SEQ ID NO:1) is encoded by the RNASET2 gene, located at the 6q27 region of the human genome (see Campomenosi et al, Arch Biochem Biophys, 2006; 449:17-26). Native RNASET2 is expressed as a pre-protein including a 24 amino acid signal sequence (see FIG. 1 of Thorn et al., 2012), which is cleaved during secretion to yield the mature human RNASET2 (SEQ ID NO: 1). Native mature human RNASET2 is a transferase-type endoribonuclease with an acidic pH optimum, which hydrolizes RNA without base specificity. Like other members of the T2 family, native human RNASET2 is effective in inhibiting pollen tube elongation, binds actin and has anti-clonogenic, anti-angiogenic, anti-tumorigenic and anti-metastatic activity distinct from and independent of its ribonucleolytic function.

According to some embodiments, the core sequence comprises at least 10 amino acids of helix 5 of naturally occurring homologues of human RNASET2. As used herein, the term “naturally occurring homologue” or “homolog” relates to a homologous protein or nucleic acid sequence encoding the protein which occurs in nature, and is similar in structure and/or sequence and/or function to the reference protein. Naturally occurring homologues of human RNASET2 retain similar or identical function to that of the reference sequence, for example, ribonucleolytic activity, actin binding, anti-tumorigenic, anti-metastatic and anti-angiogenic activity. In some embodiments, the naturally occurring homolog has both actin binding and ribonucleolytic activity. The homolog may be a human sequence or a non-human sequence.

It will be appreciated that the T2 RNase family comprises a large number of members from vastly diverse bacterial, fungal, protozoan, plant and animal species, similar in function and three-dimensional structure which share at least 40-60% or more sequence homology to the human RNASET2 protein. Homology to human RNASET2 can be determined by homology of structure (i.e. three dimensional conformation), as well as, and optionally in conjunction with amino acid sequence and functional homology. In some embodiments, the naturally occurring homologues of the human RNASET2 comprise any one or more of the non-human T2 RNase or related polypeptides. A non-exhaustive list of naturally occurring homologues of human RNASET2 is provided in Table 1.

TABLE 1
T2 RNase Homologues

| | Taxonomic | | | Human | |
|---|---|---|---|---|---|
| Organism | classification | Gene | Description | Similarity | Details |
| gorilla | Mammalia | RNASET2 | ribonuclease T2 | 99% | Gene |
| (Gorilla gorilla) | | | | identity(a) | ID101136020 |
| | | | | | GenBank NUCL |
| | | | | | XM_004044947.1 |
| | | | | | GenBank Prot |
| | | | | | XP_004044995 |
| chimpanzee | Mammalia | RNASET2 | ribonuclease T2 | 99.61(n) | GeneID100971822 |
| (Pan troglodytes) | | | | 99.22(a) | GenBank NUCL |
| | | | | | XM_003820224.1 |
| | | | | | GenBank Prot |
| | | | | | XP_003820272.1 |
| chimpanzee | Mammalia | RNASET2 | ribonuclease T2 | 99% | Gene |
| (Pan paniscus) | | | | identity(a) | ID100971822 |
| | | | | | GenBank NUCL |
| | | | | | XM_0038202724.1 |
| | | | | | GenBankProt |
| | | | | | XP_003820272.1 |
| mouse | Mammalia | Rnaset2a | ribonuclease T2A | 75.85(n)¹ | Gene |
| (Mus musculus) | | | | 69.29(a)¹ | ID100037283 |
| | | | | | GenBankNUCL |
| | | | | | XM_0006523276.1 |
| | | | | | GenBankProt |
| | | | | | XP_006523339.1 |
| hamster | Mammalia | Rnaset2a | ribonuclease T2A | 69% | GenBankProt |
| (Cricetulus griseus) | | | | identity(a) | EGV69686 |
| galago | Mammalia | Rnaset2a | ribonuclease T2A | 83% | Gene ID |
| (Otolemur garnetti) | | | | identity(a) | 100962483 |
| | | | | | GenBank NUCL |
| | | | | | XM_0037924261 |
| | | | | | GenBank Prot |
| | | | | | XP_003792469 |
| rat | Mammalia | Rnaset2 | ribonuclease T2 | 75.44(n) | Gene ID 292306 |
| (Rattus norvegicus) | | | | 69.64(a) | GenBank NUCL |
| | | | | | BC168957 cds |
| | | | | | GenBankProt |
| | | | | | AAI68957.1 |
| horse | Mammalia | Rnaset2 | ribonuclease T2 | 72% | Gene ID |
| (Eqqus caballus) | | | | identity(a) | 100055381 |
| | | | | | GenBankNUCL |
| | | | | | XM_005608131.1 |
| | | | | | GenBankProt |
| | | | | | XP_005608188.1 |
| cow | Mammalia | RNASET2 | ribonuclease T2 | 71.34(n) | GeneID |
| (Bos taurus) | | | | 62.5(a) | 508245 |
| | | | | | GenBankNUCL |
| | | | | | NM_001206337.1 |
| | | | | | GenBank Prot |
| | | | | | NP_001193266.1 |
| dog | Mammalia | RNASET2 | ribonuclease T2 | 77.1(n) | Gene ID612451 |
| (Canis familiaris) | | | | 75.2(a) | GenBankNUCL |
| | | | | | XM_005615571.1 |
| | | | | | GenBank Prot |
| | | | | | XP_003792469.1 |
| cat | Mammalia | RNASET2 | ribonuclease T2 | 69% | Gene |
| (Felis catus) | | | | identity(a) | ID101080386 |
| | | | | | GenBankNUCL |
| | | | | | XM_003986718.1 |
| | | | | | GenBank Prot |
| | | | | | XP_003986767 |
| pig | Mammalia | Rnaset2 | ribonuclease T2 | 68% | Gene |
| (Sus scrofa) | | | | identity(a) | ID100157985 |
| | | | | | GenBankNUCL |
| | | | | | XM_001928085.3 |
| | | | | | GenBank Prot |
| | | | | | XP_001928120.2 |
| oppossum | Mammalia | — | Uncharacterized | 64(a) | GeneID 100032595 |
| (Monodelphis domestica) | | | protein | | GenBank NUCL |
| | | | | | XM 001381533 |
| | | | | | GenBank Prot |
| | | | | | XP 001381570 |
| platypus | Mammalia | | Uncharacterized | 59(a) | GeneID 100077456 |
| (Ornithorhynchus | | | protein | | GenBank NUCL |
| anatinus) | | | | | XM 001508626.2 |
| | | | | | GenBank Prot |
| | | | | | XP 001508677 |
| chicken | Aves | RNASET2 | ribonuclease T2 | 62.67(n) | GeneID421569 |
| (Gallus gallus) | | | | 55.6(a) | GenBankNUCL |
| | | | | | NM_001039491.1 |
| | | | | | GenBankProt |
| | | | | | NP_001034580.1 |
| lizard | Reptilia | — | Uncharacterized | 47(a) | GeneID |
| (Anolis carolinensis) | | | protein | | 1000566146 |
| | | | | | GenBank NUCL |
| | | | | | XM 003215895.1 |
| | | | | | GenBank Prot |
| | | | | | XP 003215943 |
| African clawed frog | Amphibia | Rnase T2 | Ribonuclease T2 | 79.95(n) | GeneID 446418 |
| (Xenopus laevis) | | | | | GenBank NUCL |
| | | | | | NM 001093114.1 |
| | | | | | GenBank Prot |
| | | | | | XP 001086583 |
| tropical clawed frog | Amphibia | Rnase T2 | Ribonuclease T2 | 78.59(n) | GeneID 780173 |
| (Xenopus tropicalis) | | | | | GenBank NUCL |
| | | | | | XM 001079248 |
| | | | | | GenBank Prot |
| | | | | | XP 001072716 |
| rattlesnake | Reptilia | Rnase T2-like | Ribonuclease T2 | 53%(a) | GenBank Prot |
| (Crotalus adamantus) | | | | | AFJ51166.1 |
| zebrafish | Actinopterygii | rnaset2 | ribonuclease T2 | 56.84(n) | Gene ID791890 |
| (Danio rerio) | | | | 50.72(a) | GenBank NUCL |
| | | | | | NM_001030064.1 |
| | | | | | GenBank Prot |
| | | | | | NP_001025235.1 |
| Salmon | Teleostei | T2 RNase | RNase Ok2 | 47%(a) | GenBank NUCL |
| (Onchorhynclus keta) | | | | | AB061717.1 |
| | | | | | GenBank Prot |
| | | | | | BAB55596.1 |
| sea squirt | Ascidiacea | | Uncharacterized | 31(a) | GeneID 100182567 |
| (Ciona intestinalis) | | | protein | | GenBank NUCL |
| | | | | | XM 002129609.2 |
| | | | | | GenBank Prot |
| | | | | | XP 002129645 |
| fruit fly | Insecta | RNaseX25 | Ribonuclease X25 | 47.29(n) | Gene ID38885 |
| (Drosophila melanogaster) | | | | 34.88(a) | GenBankNUCL |
| | | | | | NM_079242.2 |
| | | | | | GenBankProt |
| | | | | | NP_523966.2 |
| mosquito | Insecta | AgaP_AGAP009842 | AGAP009842-PA | 46.17(n) | Gene ID1279260 |
| (Anopheles gambiae) | | | | 35.63(a) | GenBankNUCL |
| | | | | | XM_318955.3 |
| | | | | | GenBankProt |
| | | | | | XP_318955.3 |
| worm | Secernentea | K10C9.3 | Protein K10C9.3 | 45.44(n) | Gene ID 190096 |
| (Caenorhabditis elegans) | | | | 32.34(a) | GenBankNUCL |
| | | | | | NM_070969.3 |
| | | | | | GenBankProt |
| | | | | | NP_503370.1 |
| baker's yeast | Saccharomycetes | RNY1(YPL123C) | Vacuolar RNase | 31%(a) | Gene ID855980 |
| (Saccharomyces cerevisiae) | | | of the T2 family | | GenBank Prot |
| | | | | | NP_015202.1 |
| thale cress | Eudicotyledons | RNS3 | ribonuclease 3 | 44.38(n) | GeneID839225 |
| (Arabidopsis thaliana) | | | | 33.73(a) | GenBankNUCL |
| | | | | | NM_102446.2 |
| | | | | | GenBank Prot |
| | | | | | NP_564264.1 |
| tomato | Solanicea | RNase LE | Ribonuclease T2 | 31%(a) | Gene ID 544098 |
| (Solanum lycopersicum) | | | | | GenBankNUCL |
| | | | | | NM_001247266 |
| | | | | | GenBank Prot |
| | | | | | NP_001234195.1 |
| bitter gourd | Eudicotyledons | RNase MC1 | Ribonuclease T2 | 29%(a) | GenBank Prot |
| (Momordica charantie) | | | | | P23540 |
| tobacco | Eudicotyledons | RNase NW | Ribonuclease T2 | 30%(a) | GenBank NUCL |
| (Nicotiana glutinosa) | | | | | (cds) |
| | | | | | AB112028 |
| | | | | | GenBank Prot |
| | | | | | BAC77613.1 |
| bindweed | Eudicotyledons | RNase T2 | Ribonuclease T2 | 27%(a) | GenBank |
| (Calystegia sepium) | | | | | NUCL(cds) |
| | | | | | AF139660 |
| | | | | | GenBank Prot |
| | | | | | AAF45022.1 |
| pear | Eudicotyledons | S3-RNase | Ribonuclease T2 | 28%(a) | GenBank |
| (Pyrus pyrifolia) | | | | | NUCL(cds) |
| | | | | | AB025421 |
| | | | | | GenBank Prot |
| | | | | | BAA93052 |
| rice | Liliopsida | Os08g0434100 | hypothetical | 47.02(n) | Gene ID4345656 |
| (Oryza sativa) | | | protein | 35.27(a) | GenBankNUCL |
| | | | | | NM_001068412.1 |
| | | | | | GenBank Prot |
| | | | | | NP_001061877.1 |
| fungus | Ascomycota | RNase B1 | Ribonuclease T2 | 35%(a) | Gene ID 9977729 |
| (Aspergillus niger) | | (ACTIBIND) | | | GenBank NUCL |
| | | | | | (cds) |
| | | | | | DQ115376.1 |
| | | | | | GenBank Prot |
| | | | | | AAZ22530.1 |
| fungus | Zygomycota | RNase Rh | Ribonuclease T2 | 28%(a) | GenBankNUCL |
| (Rhizopus niveus) | | | | | (cds) |
| | | | | | D12476.1 |
| | | | | | GenBank Prot |
| | | | | | BAA02042.1 |
| bacteria | Eubacteria | RNase I | Ribonuclease T2 | 21%(a) | GenBankNUCL |
| (Escherichia coli) | | | | | EU904439.1 |
| | | | | | GenBank Prot |
| | | | | | ACI86985 |

Partial sequence alignment, including helix 5 and flanking regions, of human RNASET2 and naturally occurring homologues thereof in which the presence of alpha-helical structure corresponding to helix 5 has been identified is provided in Table 2.

TABLE 2
Helix 5 and Helix 6 Homologues
Vertebrates

| | |
|---|---|
| PRIMATES | |
| Homo sapiens | KKYFGRSLELYRELDLNSVLLKLGIK |
| (SEQ ID NO: 57) | |
| Gorilla gorilla | KKYFGRSLELYRELDLNSVLLKLGIK |
| Pan paniscus | KKYFGRSLELYRELDLNSVLLKLGIK |
| Otolemur garnettii | KKYFGKSLALYQKLDLNSVLLKLGIK |
| (SEQ ID NO: 105) | |
| OTHER MAMMALS | |
| Canis lupus | KKYFGGSLDLYRDLDLNSMLQKLGIK |
| (SEQ ID NO: 106) | |
| Equus caballus | KKYFGKSLDLYKELSLNSMLQKLGIK |
| (SEQ ID NO: 107) | |
| Sus scrofa | -KYFGKTLDLYKELALNSTLQKLGIK |
| (SEQ ID NO: 108) | |
| Felis catus | KRYFGGGLDLYQKLALNSMLQKLGIK |
| (SEQ ID NO: 109) | |
| (RODENTS) | |
| Cricetulus griseus | KKYFGKSLDLYKQLDLNSVLLKFGIK |
| (SEQ ID NO: 110) | |
| Mus musculus | KKYFGKSLDLYKQIDLNSVLQKFGIK |
| (SEQ ID NO: 111) | |
| Rattus norvegicus | -KYFGKSLDLYKQIDLNSVLQKFEIK |
| (SEQ ID NO: 112) | |
| BIRDS | |
| Gallus gallus | KKYFSKTLELYQLVNLNGFLLKAGIK |
| (SEQ ID NO: 113) | |
| REPTILES | |
| Crotalus adamanteus | KKYFQKALELYRKIDLNSFLLKVGIK |
| (SEQ ID NO: 114) | |
| Anolis carolinensis | KKYFNKALELYKKLDLNSYLLKLGIK |
| (SEQ ID NO: 115) | |
| AMPHIBIANS | |
| Xenopus laevis | -KYFSKGLEIYKQVDLNSVLEKSGI |
| (SEQ ID NO: 116) | |
| FISH | |
| Danio rerio | -KYFGKALELYHKFDLNSVLLK---- |
| (SEQ ID NO: 117) | |
| Oncorhynchus keta | -KYFGKVLELYHMVDLDGVMKKFNI |
| (SEQ ID NO: 118) | |

Plants, Fungi, Bacteria

| | |
|---|---|
| Aspergillus niger | EEVQDFFQQVVDLFKTLDSYTALSDAGIT |
| (SEQ ID NO: 119) | |
| Rhizopus niveus | EDIVDYFQKAMDLRSQYNVYKAFSSN |
| (SEQ ID NO: 120) | |
| Escherichia coli | DAYFGTMVRLNGEIKESEAGKFLAD |
| (SEQ ID NO: 121) | |
| Lycopsersicum | QHAYFKKALDLKNGIDLLSILGGA |
| esculentum | |
| (SEQ ID NO: 122) | |
| Momordica charantie | QAAYFKLAVDMRNNYSDIIGALRPHAAG |
| (SEQ ID NO: 123) | |
| Nicotiana glutinosa | QHQYFKKALDLKNQINLLEILQQAQINP |
| (SEQ ID NO: 124) | |
| Calystegia sepium | QYEYFSTTLMLYFKYNISEILSES |
| (SEQ ID NO: 125) | |
| Pyrus pyrifolia | ENHYFETVIKMYISKKQNVSRILSKA |
| (SEQ ID NO: 126) | |

1. The human helix 5 is underlined. Helix 6 is double-underlined. The last four amino acids of the human sequence at the C-terminal sequence are in random coil.
2. In invertebrates, helix 5 is underlined, and helix 6 is double underlined.
3. In invertebrates, the linker sequence is italicized.

A non-exhaustive list of homologues of helix 5 of human RNASET2 from naturally occurring homologues of human RNASET2, suitable for inclusion as helix 5 homologues in the isolated peptide of the invention comprises SEQ ID NOs. 3-24.

The term “core amino acid sequence”, as used herein, relates to an amino acid sequence which is found in all of the isolated actin binding peptides of the invention. According to a specific embodiment of the invention, the core amino acid sequence of the isolated peptides of some embodiments of the invention can be critical to the actin-binding function of the isolated peptides of the invention, for example, by conferring a three dimensional conformation allowing intimate molecular interaction between specific amino acid residues of the peptide and those of the actin molecule, of sufficient strength to form a high affinity complex. In some embodiments, the core amino acid sequence comprises at least 10 amino acids of helix 5 of human RNASET2 protein.

Surprisingly, when a peptide array comprising over 190 overlapping human RNASET2 peptide fragments was screened for actin binding (using actin and detected with rabbit anti-actin and peroxidase-conjugated anti-IgG), significant actin binding was observed with a few shorter peptides (e.g. SEQ ID NOs. 62, 131 and 132) from within the helix 5 domain of human RNASET2 (see Example II, below).

As used herein, “helix 5 of the human RNASET2 protein” refers to the amino acid sequence comprising amino acid positions 107-121 of the mature, native RNASET2 protein (SEQ ID NO: 2). When comprised within the whole human RNASET2 polypeptide, and under substantially physiological conditions, the helix 5 sequence of the RNASET2 protein forms an alpha-helical three-dimensional configuration (see Thorn et al). However, it will be noted that when comprised within isolated peptides having the core amino acid sequence, or sequences homologous or heterologous to helix 5 of the human RNASET2, the core sequence may not retain the helical conformation as in the isolated peptide. Thus, in some embodiments, the core amino acid sequence comprises at least one sequence having an alpha-helical three-dimensional conformation in the isolated peptide, while in other embodiments, the core amino acid sequence lacks sequences having an alpha-helical three dimensional conformation in the isolated peptide.

In some embodiments, the core amino acid sequence comprises at least 10, between 10-12, between 10-15 or 15 amino acids of helix 5 of human RNASET2 polypeptide, within the amino acid sequence of coordinates 107-121 of the human RNASET2 polypeptide. In other embodiments, the core amino acid sequence comprises at least 10, between 10-12, between 10-15, 10-18, 18, 10-20 or more amino acids of a helix from a homologous (e.g., naturally occurring, non-human) T2 RNase, the helix corresponding to the position of human RNASET2 helix 5 in the three dimensional conformation.

In one embodiment, the at least 10 amino acids of helix 5 of human RNASET2 correspond to positions 108-121 of SEQ ID NO: 1. In another embodiment, the at least 10 amino acids of helix 5 of human RNASET2 correspond to positions 109-121 of SEQ ID NO: 1. In yet another embodiment, the at least 10 amino acids of helix 5 of human RNASET2 correspond to positions 110-121 of SEQ ID NO: 1. In another embodiment, the at least 10 amino acids of helix 5 of human RNASET2 correspond to positions 111-121 of SEQ ID NO: 1. In some embodiments the core amino acid sequence comprises SEQ ID NO: 127 (Y110-E120 of SEQ ID NO: 1).

According to some embodiments, the isolated T2 RNase peptide of the invention comprises, in addition to the core amino acid sequence comprising at least 10 amino acids of helix 5 of SEQ ID NO: 1, at least one additional amino acid sequence. The at least one additional amino acid sequence can comprise a human RNASET2 sequence or a T2 RNase sequence homologous to the human RNASET2 sequence. The at least one additional sequence can comprise native and/or naturally occurring T2 RNase sequences, or conservative amino acid substitutions.

As used herein, the phrase “homologous additional amino acid sequence” refers to an additional sequence flanking (C-terminally or N-terminally) the core amino acid sequence in native human RNASET2. Thus, in some embodiments, the at least one additional amino acid sequence can be a homologous sequence to the core amino acid sequence—particularly, the additional amino acid sequence can be a sequence corresponding to a region or regions of human RNASET2 contiguous with the core amino acid sequence. In some embodiments, the additional amino acid sequence comprises an amino acid sequence corresponding to human RNASET2 amino acid positions flanking the region of the core amino acid sequence (N-terminally or C-terminally). In other embodiments, the additional amino acid sequence comprises portions of more than one amino acid sequence corresponding to human RNASET2 amino acid positions flanking the region of the core amino acid sequence. It will be appreciated that these homologous additional amino acid sequences are defined relative to the position of the core amino acid sequence.

In some embodiments, the isolated polypeptide comprises at least two additional amino acid sequences, flanking the core amino acid sequence of the peptide (e.g. one additional sequence at the N-terminal side of the core sequence and another, identical, or non-identical additional sequence at the C-terminal side of the core amino acid sequence).

In some embodiments, the additional amino acid sequence comprises a human RNASET2 amino acid sequence selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36, a portion thereof, or conservative substitutions thereof. In some embodiments, the at least one additional amino acid sequence is of a non-human T2 RNase sequence or conservative amino acid sequences thereof, homologous to regions of the human RNASET2 contiguous with the region corresponding to the core amino acid sequence.

The homologous at least one additional amino acid sequence can be a sequence of the same species as that of the core sequence, for example, where the core sequence corresponds to a human RNASET2 sequence, the additional amino acid sequence can comprise sequences from human RNASET2 as detailed herein. Yet further, the at least one additional amino acid sequence can comprise sequences heterologous to the core amino acid sequence, particularly, sequences corresponding to regions not contiguous with region or regions of human RNASET2 or heterologous amino acid sequences from naturally occurring homologues of human RNASET2. Such heterologous sequences may comprise human RNASET2 sequences, or sequences of naturally occurring homologues or conservative substitutions thereof from remote regions of the RNASET2 polypeptide, or other discontinuous (non-contiguous) portions of human RNASET2, or of naturally occurring RNASET2 homologues, or conservative substitutions thereof. In some embodiments, the at least one additional sequence can comprise partly or wholly synthetic amino acid sequences.

It will be appreciated that the isolated peptide, when comprising one or more heterologous additional amino acid sequences, is a fusion peptide or a chimeric peptide. As used herein, the term “fusion peptide” or “chimeric peptide” relates to a peptide, the amino acid sequence of which comprises combinations of a core amino acid sequence, and amino acid sequences not naturally occurring contiguously with the core sequence in nature. In some embodiments, the core amino acid sequence and the additional amino acid sequence(s) of the fusion or chimeric peptide are synthesized together as one single synthetic peptide. In some embodiments, the component portions of the fusion or chimeric peptide are fused by chemical fusion, to produce the chimeric peptide. Yet further, in some embodiments, a nucleic acid sequence encoding the fusion or chimeric peptide of the invention is designed such that the chimeric or fusion peptide can be produced by genetic engineering of a host cell to express and, optionally, release the fusion or chimeric peptide into the medium.

In another embodiment of the invention, the fusion or chimeric peptide of the invention comprises one or more additional amino acid sequences having conservative amino acid substitutions.

Many of the actin binding peptide sequences derived from human RNASET2 include regions corresponding to helix 5 and helix 6 of human RNASET2. Without wishing to be limited to a single hypothesis, one explanation could be that such a configuration is important for actin binding of the peptide. Thus, in some embodiments, the at least one additional amino acid sequence is a sequence corresponding to an alpha helix in human RNASET2, particularly, corresponding to helix 6 (amino acids 123-129 of RNASET2, SEQ ID NO: 37) or helix 7 (amino acids 141-152 of RNASET2, SEQ ID NO: 38) of human RNASET2. In some embodiments, the additional amino acid sequence corresponds to helix 6 of human RNASET2, or a portion thereof.

Where the additional amino acid sequence is a helix, or corresponds to a helix of a T2 RNase, the additional amino acid sequence can also be selected from homologues of helix 6 or 7 of human RNASET2. A non-exhaustive list of homologues of helix 6 of human RNASET2 from naturally occurring homologues of human RNASET2, suitable for inclusion as additional amino acid sequences in the isolated peptide of the invention comprises SEQ ID NOs. 39-56.

Prediction of three-dimensional conformation, for example, to determine the likelihood of helical or non-helical configuration of a peptide, polypeptide or peptide fragment, or to predict likely binding sites, based on approximation of docking of two or more molecules can be performed using X-ray crystallography data, bioinformatics databases and model-building algorithms. Commonly used programs include Agadir, APSSP, CFSSP, GOR, Marcoil, Open Structure, Procheck available from ExPasy (Swiss Institute of Bioinformatics), and CBLAST, Cn3D, VAST and MMDB available from NCBI (National Center for Biotechnology Information, USA).

In some embodiments, the at least one additional amino acid sequence comprises a linker sequence. In some embodiments, the linker sequence is positioned between the core amino acid sequence and a homologous or heterologous additional helical human RNASET2 sequence. In some embodiments the linker comprises 1-9, 3-7, 4-6, or 1, 2, 3, 4, 5, 6, 7, 8, 9 or more amino acids. In a particular embodiment the linker comprises 2 or 3 amino acids, with at least one of the amino acids being a charged amino acid. In some embodiments, the linker comprises negatively charged amino acids. In some embodiments, the linker comprises neutral and negatively charged amino acids, with a negatively charged amino acid at a position corresponding to amino acid coordinate 122 of SEQ ID NO: 1. In some embodiments, the linker sequence is Aspartic acid. In other embodiments, the linker comprises Leucine-Aspartic acid, Aspartic acid-Leucine or Leucine-Aspartic acid-Leucine.

In other embodiments, the core amino acid sequence comprises a T2 RNase sequence comprising conservative amino acid substitutions.

As used herein, the phrase “conservative amino acid substitutions” relates to substitutions of amino acids within groups defined by similar structure, polarity and charge. As used herein, conservative substitutions of the naturally occurring 20 amino acids comprise substitutions within the following groupings:

(A) Small aliphatic, nonpolar or slightly polar residues: Ala, Ser, Thr, Pro and Gly; (B) polar, negatively charged residues and their (uncharged) amides: Asp, Asn, Glu and Gln; (C) polar, positively charged residues: His, Arg and Lys; (D) large aliphatic, nonpolar residues: Met, Leu, Ile, Val and Cys; and (E) aromatic residues: Phe, Tyr and Trp.

More specifically, conservative substitutions comprise substitution of Ala into Gly or into Ser; Arg into Lys; Asn into Gln or into His; Asp into Glu; Cys into Ser; Gln into Asn; Glu into Asp; Gly into Ala or into Pro; His into Asn or into Gln; Be into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Be or into Leu.

However, it will be appreciated that in the context of helical three dimensional conformation, substitution of Proline for another member of group A may affect the tendency of a peptide to helical structure, and thus, in some embodiments, may not be considered a conservative substitution within group A.

According to some embodiments, the isolated peptide is 23-50, 23-45, 23-40, 23-35, 23-30, 23-27, 25-45, 25-40, 30-45 or 30-40 amino acids in. In some embodiments, the isolated peptide is 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

Comparison of the human and non-human T2 RNase amino acid sequences corresponding to the area of helix 5 and its flanking regions has revealed conservation of a few of the charged amino acids in the region corresponding to amino acid positions 112-124 of SEQ ID NO: 1 (see, for example, Table 2). Thus, according to a specific embodiment presence of charged amino acid side-chains may be important to or critical to the structure and/or function (i.e. actin binding) of the isolated peptide.

Thus, according to one aspect of one embodiment of the invention, amino acids of the core amino acid sequence corresponding to positions 116 and 122 of SEQ ID NO: 1 are negatively charged amino acids. In another embodiment, the amino acids of the core amino acid sequence corresponding to positions 116 and 122 of SEQ ID NO: 1 are negatively charged amino acids and the amino acid of the core amino acid sequence corresponding to position 119 of SEQ ID NO: 1 is a positively charged amino acid. Negatively charged amino acids include, but are not limited to Glu and Asp. Positively charged amino acids include, but are not limited to Arg and Lys. Some non-conventional negatively and positively charged amino acids are included in Table 3 herein.

In some embodiments, the isolated peptide of the invention comprises the amino acid sequence:

X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃; (SEQ ID NO: 128) wherein X₁ and X₈ are selected from group E; X₂, X₄, X₁₅ and X₂₁ are selected from group A; X₃, X₉, X₁₉ and X₂₃ are selected from group C; X₅, X₇, X₁₁, X₁₃, X₁₆, X₁₇, X₁₈, X₂₀ and X₂₂ are selected from group D and X₆, X₁₀, X₁₂ and X₁₄ are selected from group B; wherein group A consists of small, aliphatic, non-polar or slightly polar amino acid residues, group B consists of polar, negatively charged amino acid residues and their (uncharged) amides; group C consists of polar, positively charged amino acid residues, group D consists of large, aliphatic non-polar amino acid residues and group E consists of aromatic residues. In other embodiments, group A comprises an amino acid sequence selected from the group consisting of amino acids Ala, Ser, Thr, Pro and Gly; group B consists of amino acids Asp, Asn, Glu and Gln; group C consists of amino acids His, Arg and Lys; group D consists of amino acids Met, Leu, Ile, Val and Cys and group E consists of amino acids Phe, Tyr and Trp. It will be noted that, in specific embodiments, Proline can optionally be excluded from group A due to potential disruption of helical structure.

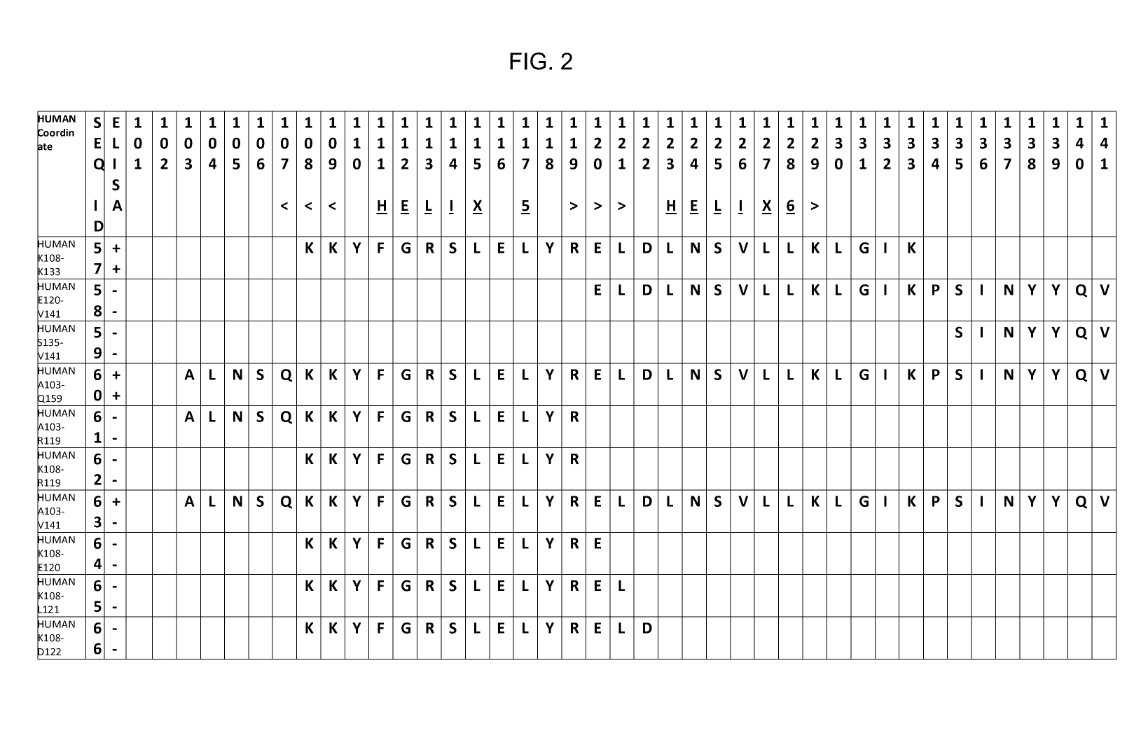

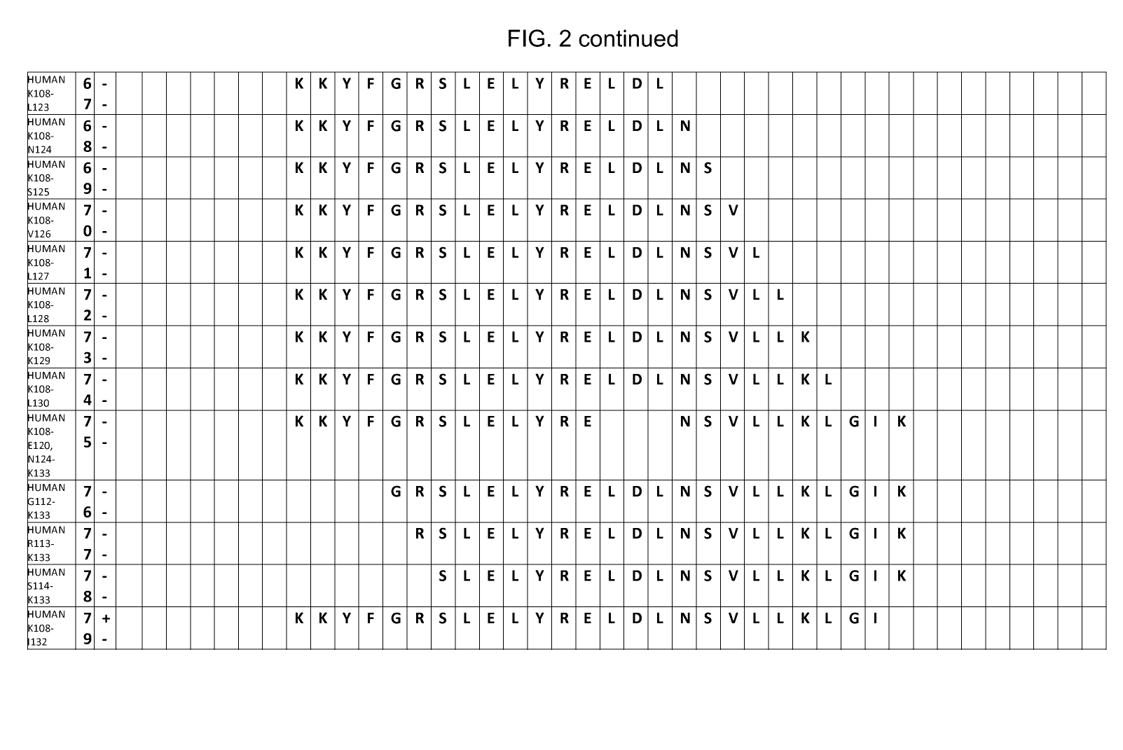

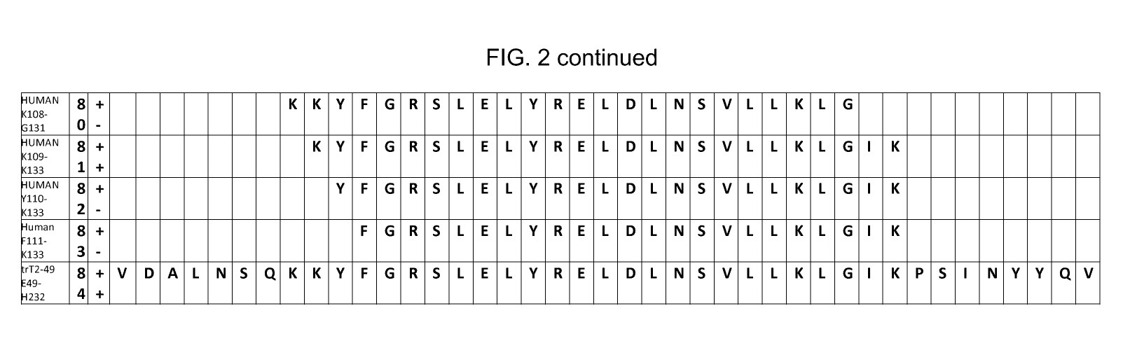

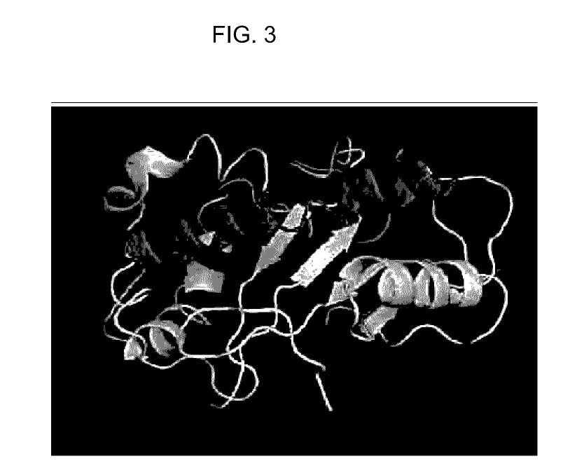

FIG. 2 provides a comparison of RNASET2 peptides corresponding to the region of human RNASET2 comprising helix 5 and helix 6, as well as some flanking regions (SEQ ID NOs. 57-84). ELISA analysis of actin binding of the isolated peptides in FIG. 2 illustrates the actin binding properties of RNASET2 peptides comprising the core amino acid sequence of the present invention. In specific embodiments, the isolated T2 RNase peptide is selected from the group consisting of SEQ ID NOs. 57 and 79-83.

In some embodiments, the isolated peptide of the invention further comprises the amino acid X₀ positioned N-terminally to X₁, wherein X₀ is selected from group E. In further embodiments, the isolated peptide of the invention further comprises the amino acids X_{0′}X₀ positioned N-terminally to X₁, wherein X_{0′} is selected from group C and X₀ is selected from group E. In yet further embodiments, the isolated peptide of the invention further comprises the amino acids X_{0″}X_{0′}X₀ positioned N-terminally to X₁, wherein X_{0′} and X_{0″} are selected from group C and X₀ is selected from group E.

In some embodiments, the core amino acid sequence is as set forth in SEQ ID NO: 83. In some embodiments, the core amino acid sequence is as set forth in SEQ ID NO: 82. In some embodiments, the core amino acid sequence is as set forth in SEQ ID NO: 81. In some embodiments, the core amino acid sequence is as set forth in SEQ ID NO: 57. In some embodiments, the core amino acid sequence is as set forth in SEQ ID NO: 130. In some embodiments, the core amino acid sequence is as set forth in SEQ ID NO: 133.

As used herein, the term “isolated” refers to a protein, polypeptide or peptide removed from its normal physiological context. In some embodiments, the term “isolated” refers to a peptide substantially free (at least 90% of the solution comprises a protein content consisting of the peptide of some embodiments of the invention) of cellular material (e.g., proteins other than T2 RNase) or culture medium when produced by recombinant DNA techniques, or chemical precursors or other chemicals when chemically synthesized.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc), for example, the peptides of the invention may include D-amino acids, e.g. SEQ ID NO: 130.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The isolated peptide can comprise natural amino acids, synthetic amino acids or both naturally occurring and synthetic amino acids. Thus, according to some embodiments, the isolated peptide of the invention comprises at least one synthetic amino acid. In some embodiments, the isolated peptide of the invention comprises at least 2, at least three, at least 4, 5, 8, 10 or more synthetic amino acids.

Tables 3 and 4 below list naturally occurring amino acids (Table 3), and non-conventional or modified amino acids (e.g., synthetic, Table 4) which can be used with some embodiments of the invention.

TABLE 3

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 4

| Non-conventional amino acid | Code | Non-conventional amino acid | Code |
|---|---|---|---|
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline-3- | Tic |
| ethylamino)cyclopropane | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention may be utilized in a linear form, however cyclic forms of the peptide can also be utilized.

Since the present peptides are utilized in therapeutics or diagnostics which may require the peptides to be in soluble form, the peptides of some embodiments of the invention may include one or more non-natural or natural polar amino acids, including but not limited to serine and threonine which are capable of increasing peptide solubility due to their hydroxyl-containing side chain.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

One method of preparing the peptide compounds of some embodiments of the invention involves solid phase peptide synthesis.

Large scale peptide synthesis is described by Andersson, Biopolymers 2000; 55(3):227-50.

The human RNASET2 peptide can be recombinantly produced by expressing a polynucleotide encoding same, using an appropriate expression vector system. Thus, according to one embodiment there is provided an isolated polynucleotide encoding a peptide of the present invention as described herein e.g., a human RNASET2 peptide 23-50 amino acids in length and capable of binding actin. Exemplary polynucleotides encoding the human RNASET2 peptide include, but are not limited to polynucleotides comprising SEQ ID NO: 85, SEQ ID NO: 86, SEQ ID NO: 87 and SEQ ID NO: 88, encoding the peptide sequences SEQ ID NO: 57, SEQ ID NO: 81, SEQ ID NO: 82 and SEQ ID NO: 83, respectively, and polynucleotides encoding SEQ ID NO: 133.

As such, the term “polynucleotide” when used herein in context of T2 RNase peptide in general, or in context of any specific T2 RNase peptide, refers to any polynucleotide sequence which encodes a T2 RNase peptide having actin-binding activity.

The term “nucleic acid” refers to polynucleotides or to ologonucleotides such as deoxyribonucleic acid (DNA), and, where appropriate, ribonucleic acid (RNA) or mimetics thereof. The term should also be understood to include, as equivalents, analogs of either RNA or DNA made from nucleotide analogs, and, as applicable to the embodiment being described, single (sense or antisense) and double-stranded polynucleotides.

Thus, in one embodiment, the polynucleotide is at least 75%, at least 80%, at least 85%, at least 88%, at least 90%, at least 93%, at least 95%, at least 98%, and 100% homologous to SEQ ID NO: 85. In a yet further embodiment, the polynucleotide is as set forth in SEQ ID NOs: 85-88.

As detailed in Example II, some short peptide fragments of human RNASET2 polypeptide sequence exhibit significant actin binding activity, for example, when immobilized on a solid support and/or matrix. Thus, according to another aspect of the present invention, there is provided an isolated peptide comprising the amino acid sequence as set forth in SEQ ID NO: 132, or naturally occurring homologues thereof, or conservative substitutions thereof, wherein the peptide binds actin. According to still another aspect of the present invention, there is provided a core amino acid sequence, which comprises at least 5 amino acids of helix 5 of human RNASET2, or naturally occurring homologues thereof, or conservative substitutions thereof, wherein the peptide is 5-22 amino acids in length and wherein the peptide binds actin. In yet a further embodiment of the invention the isolated peptide is selected from the group consisting of SEQ ID NOs 62, 131 and 132.

According to yet other aspects of the present invention, some peptide fragments of human RNASET2 polypeptide sequence greater than 50 amino acids in length exhibit significant actin binding activity, as well as in-vitro and in-vivo angiogenesis inhibitory activity. Thus, according to another aspect of the present invention, there is provided an isolated peptide comprising the amino acid sequence as set forth in SEQ ID NO: 60 (A103-R159 of human RNASET2), or naturally occurring homologues thereof, or conservative substitutions thereof, wherein the peptide binds actin.

DNA encoding the T2 RNase peptide is easily synthesized using standard nucleic acid synthesis techniques. Once prepared, the DNA can be ligated into expression vectors, which are then transfected into host cells. Thus, according to some aspects of some embodiments of the invention, there is provided a nucleic acid construct comprising a polynucleotide encoding any one of the T2 RNase peptides of any one of the embodiments of the invention. Constructs useful in the methods according to some embodiments of the invention may be constructed using recombinant DNA technology well known to persons skilled in the art. The constructs may be inserted into vectors, which may be commercially available, suitable for transforming into prokaryotic or eukaryotic host cells and suitable for expression of the gene of interest in the transformed cells.

The genetic construct can be an expression vector wherein the nucleic acid sequence is operably linked to one or more regulatory sequences allowing expression in the host cells. Typical regulatory sequences include, but are not limited to, promoters. The promoter may be any nucleic acid sequence functional (having transcriptional activity, capable of directing transcription) in the host cell of choice including mutant, truncated, and hybrid promoters, and may be either homologous or heterologous to the host cell.

Additional regulatory elements include, but are not limited to enhancers, transcription terminator sequences leader sequences, polyadenylation sequences, a signal peptide coding region coding for an amino acid sequence for directing the encoded polypeptide into the cell's secretory pathway and a propeptide coding region, coding for an amino acid sequence positioned at the amino terminus of a polypeptide.

Suitable host cells for recombinant expression include both prokaryotic and eukaryotic hosts. Prokaryotic hosts include a wide variety of bacterial hosts including Gram-positive and Gram-negative bacteria, which can be transformed by suitable expression vector systems including bacterial transformation with bacteriophage DNA, plasmid DNA, or cosmid DNA.

Examples of Gram-negative bacteria which can be used in accordance with the present teachings include, but are not limited to, Escherichia coli, Pseudomonas, Erwinia and Serratia. Choice of host will be made with consideration of cost of operation and optimizing cell culture densities, to provide highest product yields at reasonable expense. Bacterial expression of fully active human recombinant truncated RNASET2 has been reported previously by the present inventors (see WO 2010/049933).

The RNASET2 peptides of the invention can be expressed in a variety of eukaryotic expression vector/host systems. These include but are not limited to microorganisms such as yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., T1 or pBR322 plasmid); or animal cell systems. Eukaryotic host cells may be, but are not limited to mammalian cells (such as Chinese Hamster Ovary (CHO) cells, monkey cells, baby hamster kidney cells, cancer cells or other cells), yeast cells, insect cells and the like. Specific examples include, but are not limited to Pichia cells, insect S. frugiperda or Trichoplusia larva cells and the like. Expression of fully active human recombinant RNASET2 in Pichia cells has been reported previously by the present inventors (see WO 2006/035439, fully incorporated herein in its entirety).

Mammalian cells that are useful in recombinant peptides production include but are not limited to VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Thus, embodiments of the present invention provide for a composition-of-matter comprising bacterial or eukaryotic culture remnants and at least about 70-99%, 75-95%, 80-90%, 70%, 80%, 85%, 90%, 95%, 96%, 97%, 98%, 99% or more recombinant T2 RNase peptides. Culture remnants may be further removed for clinical applications (in vivo) using methods which are well known in the art.

It has been postulated that many of the biological activities observed for human RNASET2 (anti-clonogenic effect, anti-angiogenic effect, anti-metastatic effect, etc) are dependent on steric interaction (for example, binding) of the polypeptide with actin. Thus, the isolated peptide binds actin. As used herein, the phrase “actin binding” relates to a high affinity, high specificity interaction between an isolated peptide of the invention and an actin molecule, variant or fragment thereof. Actins suitable for assessing actin binding can comprise any eukaryotic actin, for example, mammalian actin, human actin, rabbit actin, plant actin and the like. The isolated peptide of the invention can bind G-actin, F-actin, alpha-, beta- or gamma-actin. In some specific embodiments, the isolated peptide binds plant, human or rabbit G-actin, native or recombinant actin.

Actin binding can be assessed by a variety of assays, including but not limited to solution binding assays (e.g. the EDC assay detailed herein), PAGE separation and Western blotting, filter-based assays and ELISA-based assays (as detailed herein). Actin binding assay kits are commercially available, for example, from Cytoskeleton, Inc. (Denver, Colo., USA). Exemplary assays for actin binding include, for example, the solution actin binding assay and the solid-phase actin binding, as described here in detail. In some embodiments, binding of the isolated peptide to an actin sample is assayed by an ELISA assay. In some embodiments, the ELISA assay is performed with an anti T2 RNase antibody. In some embodiments, the ELISA is performed with an anti-RNASET2 antibody directed against the whole protein or a truncated RNASET2 polypeptide lacking about the first 50 N-terminal amino acids of the human RNASET2 polypeptide. The anti-T2 RNase antibody can be a polyclonal or monoclonal antibody or active fragments (scFv, etc) thereof. Whole anti-serum, partially purified or purified (e.g. affinity purified) antibodies can be used.

Some exemplary actin binding assays include, but are not limited to:

Solid Phase Binding Assay:

Solid-phase ELISA for RNASET2 peptide binding to immobilized actin is performed as modified from Mejean 1987 (Biochem. J. 244: 571-577) and Mejean 1992 (Eur. J Biochem. 209: 555-562). An exemplary Solid Phase Binding Assay is described herein. Briefly: All steps are conducted at 37° C. Immuno-96 Microwell™ solid plates (Thermo Fisher Scientific, MA) coated with 500 ng/well of G-Actin from rabbit muscle (Molecular Probes, NY) in 100 ul of 0.05 M carbonate buffer, pH 9.5 (Sigma-Aldrich, Mo), incubated for 1 h and washed with 250 ul/well TBS (Sigma-Aldrich). The wells are blocked with 3% bovine serum albumin (BSA, Sigma-Aldrich) in 200 ul/well Tris Buffered Saline (TBS) for another 1 hour and washed with 250 ul/well TBS. The peptides are then added at concentrations of various concentrations (for example, serial dilutions giving 500, 250 or 125 ng/well) in 100 ul/well Phosphate Buffered Saline (PBS), incubated for 1 hour and washed 3 times with 250 ul/well TBS containing 0.05% Tween®20 (TBST, Sigma-Aldrich). Anti-T2 RNase (such as rabbit anti-rhtrRNASET2, Anilab, Israel, whole serum or antiserum purified by a Protein A antibody purification) is then added (if purified, at a dilution of 1:500), incubated for 1 hour and the plates washed 3 times with 250 ul/well TBST. Labeled second antibody [for example, peroxidase-conjugated affinity pure goat-anti rabbit IgG (Jackson, Pa.)] is added at a dilution of 1:5,000 in 100 ul/well TBS, incubated for 1 hour and the plates washed twice with 250 ul/well TBST. After an additional wash with 250 ul/well TBS, 1-Step Ultra TMB-ELISA (TMB-3,3′,5,5′ Tetramethylbenzidine) (Thermo-Scientific) is added (100 ul/well) and after 15-25 min of incubation, the reaction is read at A650 using Infinite F50-TECAN ELISA READER (TECAN, Austria). Each assay is performed in triplicates. All the wash steps are performed with HydroFlex-TECAN ELISA PLATE Washer (TECAN, Austria).

Another ELISA assay for actin binding can also be used. Actin (5 ug/ml) protein isolated from human platelet (Cytoskeleton, Denver, Colo., USA), is diluted with carbonate-bicarbonate buffer, pH 9.5, and coated directly onto 96-well EIA/RIA plate, incubated overnight at 4° C. The plates are blocked with 5% (w/v) BSA in PBS containing 0.25% Tween-20 (PBST) at room temperature for 1 h and subsequently incubated with hrRNASET2, at serial 1:2 dilutions in PBST overnight at 4° C. Following washing the plates are incubated with rabbit anti-RNASET2 polyclonal affinity pure antibodies (GENEMED SYNTHESIS, San Antonio, Tex., USA) 1:500 diluted in PBST at 37° C. for 1 hour. Following further wash, Peroxidase-conjugated AffiniPure goat anti-rabbit IgG (Jackson ImmunoResearch Laboratories, West Grove, Pa., USA) in TBST is added and the plates incubated as before. Following further washing, signals are generated by 1-Step Ultra TMB-ELISA solution (Thermo Scientific, Pierce Biotechnology, Rockford, Ill., USA) and then the relevant optical absorbance detected at 650 nm with an Infinite F50 multidetection microplate reader (Tecan, Grodig, Austria).

Actin-Binding Monitoring by Microscale Thermophoresis (MST):

The actin-binding capacity (Dissociation Constant, KD) of hrRNASET2 can be measured by MST using a Monolith NT.115 (Nanotemper Technologies, Germany). An exemplary MST assay is described herein. Briefly, each sample is tested at serial 1:2 dilutions, from 10 to 0.31 uM in Buffer G. Actin is labeled with Monolith NT. Protein Labeling kit RED-NHS, diluted in the Buffer G, and added to each sample. The sample mixtures are incubated for 5 min at room temperature and loaded into MST-glass capillaries for MST-Analysis. MST monitors directed movement of particles in a microscopic temperature gradient. Any change of the hydration shell of biomolecules due to changes in their structure/conformation results in a relative change of movement along the temperature gradient and reflects binding affinities and binding kinetics. Each sample was scanned and measured at 40% IR-Laser Power.

Solution Actin Binding Assay:

The solution actin binding assay can be performed as previously described (PCT WO 2006/035439, Smirnoff et al. 2006. Cancer, 107(12), 2760-2769). Actin (for example, 10 μg) is mixed with, for example, 10 μg purified candidate T2 RNase peptide and 20 μL Buffer G (2 mM Tris pH 8.0, 0.2 mM CaCl, 0.2 mM ATP). The mixture is then incubated for 30 min at room temperature, followed by addition of the cross-linking agent 1-[3-(dimethylamino)-propyl]-3-ethyl-carboimide methiodide (EDC) to a final concentration of 50 mM, and incubated for another 30 minutes. The reaction is then quenched with an equal volume of sample buffer and the cross-linked complex separated on SDS-PAGE, adapted for separation of smaller MWs such as the T2 RNase peptides of the invention. The samples from each reaction mixture are separated and stained with Coomassie Blue to visualize the proteins, as well as transfer to nitrocellulose for immunodetection with anti-T2 RNase antibody (for example, polyclonal rabbit-anti-human recombinant truncated RNASET2, Anilab—Rehovot, Israel) or anti-actin (Sigma-Aldrich Company, St. Louis, Mo.; Cat A2066). The membrane after blotting is blocked overnight at 4° C. with 5% (weight/volume) skim milk in TBS with 0.25% Tween 20 (TBST), washed twice for 10 minutes each with TBST and probed with the anti-T2 RNase antibody, or with anti-actin-IgG for actin detection. Following wash and reaction with labeled second antibody [for example, if the primary antibodies are rabbit antibodies, alkaline phosphatase goat anti rabbit-IgG (Chemicon Int., Temecula, Calif.; Cat AP132A], signals can be detected by incubation and development with standard reagents.

BIACore Assay:

BIAcore analysis can assist quantifying and understanding the kinetics of the interaction between actin and the T2 RNase peptides. The strength of a two molecule interaction is characterized by the equilibrium dissociation (binding) constant K_{D}=[P][L]/[PL], where [P] is the concentration of free protein (or peptide), [L] the concentration of ligand and [PL] the concentration of the complex. At equilibrium, K_{D} is related to the rate of complex formation (described by the association rate constant, kₐ) and the rate of breakdown (described by the dissociation rate constant, k_{d}), such that K_{D}=kₐ/k_{d}. A high affinity interaction is characterized by a low K_{D}, rapid recognition and binding by high kₐ, and stability of complex formation by low k_{d} (Bioradiations, 2008, issue 119; 18; Bio-Rad). This data is extremely valuable when modifying small-molecule to optimize binding for drug target. Using actin coupled to a biosensor chip the affinities of T2 RNase peptides can be measured.

Peptide Array Assay:

Peptide arrays are powerful tools for characterizing protein interactions and identifying specific domains involved in mediating these interactions. Peptide arrays can be useful in the specific detection of individual proteins in complex mixtures, for identifying binding sites between proteins, for determining the contribution of individual amino acid residues to binding and more.

An array of partially overlapping peptides derived from a candidate polypeptide, including, optionally, modified peptides based on the candidate peptide sequence and appropriate internal and external control peptides is synthesized and derivatized (e.g acetylated) to allow attachment to a cellulose membrane (e.g. via their C-termini through an amide bond).

The array is typically blocked (e.g. with milk) to eliminate background non-specific binding, rinsed and exposed to the ligand (e.g. actin), washed and exposed to anti-ligand antibody (e.g. anti-actin antibody), and washed. Immuno-detection of actin binding can be accomplished with for example, a labeled second antibody (e.g. anti-anti-actin IgG-labeled).

The T2 RNase peptide or peptides of the invention can be provided alone, without additional agents, or can be provided in a formulation with other agents. In some embodiments, and in order to enhance effective delivery of the T2 RNase peptide across cellular membranes, the isolated peptide is provided as a composition of matter comprising the isolated peptide formulated with a cell penetrating agent. As used herein the phrase “penetrating agent” refers to an agent which enhances translocation of any of the attached peptide across a cell membrane. According to one embodiment, the penetrating agent is a peptide and is covalently attached to the T2 RNase peptide via a peptide bond.

Typically, peptide penetrating agents have an amino acid composition containing either a high relative abundance of positively charged amino acids such as lysine or arginine, or have sequences that contain an alternating pattern of polar/charged amino acids and non-polar, hydrophobic amino acids. Suitable cell penetrating peptides include those discussed in U.S. Patent Publication No. 2007/0129305. The cell penetrating peptides can be based on known peptides, including, but not limited to, penetratins; transportans; membrane signal peptides; viral proteins (e.g., Tat protein, VP22 protein, etc.); and translocating cationic peptides. Tat peptides comprising the sequence YGRKKRRQRRR (SEQ ID NO: 89) are effective in protein translocating activity. Additionally, branched structures containing multiples copies of Tat sequence RKKRRQRRR (SEQ ID NO: 90) can translocate efficiently across a cell membrane. Variants of Tat peptides capable of acting as a cell penetrating agent are described in Schwarze, S. R. et al., Science 285:1569-1572 (1999). A composition containing the C-terminal amino acids 159-301 of HSV VP22 protein is capable of translocating different types of cargoes into cells. Translocating activity is observed with a minimal sequence of DAATATRGRSAASRPTERPRAPARSASRPRRPVE (SEQ ID NO: 91). Active peptides with arginine rich sequences are present in the Grb2 binding protein, having the sequence RRWRRWWRRWWRRWRR (SEQ ID NO: 92) and polyarginine heptapeptide (SEQ ID NO: 93). Another exemplary cell penetrating peptide has the sequence RPKKRKVRRR (SEQ ID NO: 94). Also useful are branched cationic peptides capable of translocation across membranes, e.g., (KKKK)₂GGC (SEQ ID NO: 95), (KWKK)₂GCC (SEQ ID NO: 96), and (RWRR)₂GGC (SEQ ID NO: 97). A cell penetrating peptide can comprise chimeric sequences of cell penetrating peptides, for example, transportan GALFLGFLGGAAGSTMGAWSQPKSKRKV (SEQ ID NO: 98). Other types of cell penetrating peptides are the VT5 sequences DPKGDPKGVTVTVTVTVTGKGDPKPD (SEQ ID NO: 99) unstructured peptides described in Oehlke J., Biochim Biophys Acta. 1330(1):50-60 (1997); alpha helical amphipathic peptide with the sequence KLALKLALKALKAALKLA (SEQ ID NO: 100); sequences based on murine cell adhesion molecule vascular endothelial cadherin, amino acids 615-632 LLIILRRRIRKQAHAHSK (SEQ ID NO: 101); sequences based on third helix of the islet 1 gene enhancer protein RVIRVWFQNKRCKDKK (SEQ ID NO: 102), amphipathic peptide carrier Pep-1 KETWWETWWTEWSQPKKKRKV (SEQ ID NO: 103); and the amino terminal sequence of mouse prion protein MANLGYWLLALFVTMWTDVGLCKKRPKP (SEQ ID NO: 104).

In some embodiments, and in order to enhance effective delivery of the T2 RNase peptide to specific target tissues or cells (i.e. tumor cells, abnormally proliferating cells, abnormal angiogenic endothelium, metastatic cells, etc) the isolated peptide is provided as a composition of matter comprising the isolated peptide formulated with a targeting moiety. As used herein, “targeting moieties” or “targeting agents” are any moieties or agents specific for a characteristic component of the targeted region or cell. In some embodiments the targeting moieties include proteins such as polyclonal or monoclonal antibodies, antibody fragments, or chimeric antibodies, enzymes, peptides or hormones, aptamers or sugars such as mono-, oligo- and polysaccharides. In certain embodiments of the invention, contemplated targeting moieties interact with integrins, proteoglycans, glycoproteins, receptors, or transporters. Suitable moieties include any that are specific or selective for cells of the target organ, or for structures of the target organ exposed to the circulation as a result of local pathology, such as tumors.

For example, the targeting moiety can specifically bind to a marker specifically (only) expressed on cancer cells or a marker up-regulated on cancer cells compared to normal cells. The targeting moiety may specifically bind to a cancer-specific antigen (e.g., CEA (carcinoembryonic antigen) (colon, breast, lung); PSA (prostate specific antigen) (prostate cancer); CA-125 (ovarian cancer); CA 15-3 (breast cancer); CA 19-9 (breast cancer); HER2/neu (breast cancer); .alpha.-feto protein (testicular cancer, hepatic cancer); .beta.-HCG (human chorionic gonadotropin) (testicular cancer, choriocarcinoma); MUC-1 (breast cancer); Estrogen receptor (breast cancer, uterine cancer); Progesterone receptor (breast cancer, uterine cancer); and EGFr (epidermal growth factor receptor) (bladder cancer)). In a particular embodiment, the targeting moiety is an antibody or antibody fragment immunologically specific for a surface protein on cancer cells or a surface protein expressed at higher levels (or greater density) on cancer cells than normal cells, tissues, or organs. In a particular embodiment, the targeting moiety is a ligand or binding fragment thereof for a cell surface receptor on cancer cells. In a particular embodiment, the targeting moiety specifically binds to alpha-5 beta-3 integrin cell surface receptor. In a particular embodiment, the targeting moiety is an RGD peptide or RGD mimic (see, e.g., European Patent Application EP2239329). The targeting moiety can be provided with the T2 RNase peptide of the invention as a composition, and the targeting moiety can be in molecular association with the T2 RNase peptide-covalently (i.e. as a fusion protein, or other chemical linkage) or non-covalently associated with the T2 RNase peptide of the invention. In some embodiments, the isolated T2 RNase peptide of the invention is provided with a delivery system, such as liposomes, nanoparticles and the like, and the targeting moiety or agent is incorporated into the delivery system (see, for example, melanoma-targeted nanoparticles in Vannucci et al Int J Nanomed 2012; 7:1489-509).

Members of the T2 RNase family have been shown to possess biological activity unrelated to catalysis of RNA hydrolysis, including inhibition of pollen tube elongation for pollen rejection pathways in plants, suppression of solid tumors, inhibition of angiogenesis and suppression of abnormal cell growth and development. Effects on cell growth and development include, but are not limited to inhibition of clonogenicity, reduction of metastatic potential, inhibition of metastatic spread, reduction of tumor volume, inhibition of tumor angiogenesis, inhibition of invasiveness and impeding tumor development. Further, administration of enzymatically inactivated T2 RNase has been shown to effectively inhibit tumor development and metastatic transformation in models of both newly induced cancers and in models of long standing, established disease. Exemplary, non-limiting assays for the biological activity of T2 RNase peptides include:

Pollen Tube Growth (PTG) Assay:

The candidate T2 RNase peptide was tested in vitro as previously described (Roiz L et al. 2000. J Amer Soc Hort Sci. 125:9-14) with modifications. In one exemplary PTG assay, Lilium longiflorum (var. white heaven) flowers are left to open in the lab, anthers excised and brought to complete dehiscence and pollen release for 24-48 h at room temperature. The pollen is suspended in culture medium (7% sucrose (w/v), 1.27 mM Ca(NO3)2, 0.16 mM H3BO3, 1 mM KNO3 and 3 mM KH2PO4 in water), about 40 mg/ml, vigorously stirred by vortex and centrifuged for 2 min at 13,400 rpm at room temperature to release the lipid phase from pollen grains. The cell pellet is suspended in 1 ml culture medium. The procedure is repeated three times, then cells counted and suspended in culture medium up to 25,000 cells/ml. Pollen is then germinated with or without T2 RNase (for example, hrRNASET2) or T2 RNase peptides or PBS (as control), and incubated for 1-2 h at 25° C. in the dark, for maximum growth. The pollen tubes are then fixed and stained with Alexander stain (20 mg malachite green, 100 mg acid fuchsin, 5 gr phenol, 2 ml lactic acid, 20 ml ethanol, 40 ml glycerol, 50 ml water). Pollen tube length is measured microscopically, digitalized and analyzed using NIS-Elements Br software (Nikon, Japan). N=3 and at least 100 pollen tubes are measured in each set of determinations.

Cell Invasion Assay Through Matrigel™:

Highly metastatic human melanoma A375SM cells are maintained in minimum essential medium (MEM) containing fetal bovine serum, nonessential amino acids, HEPES buffer, and antibiotics. Assay of the effect of T2 RNase peptides on cell invasiveness is carried out in a 24-well BD BioCoat™ Matrigel™ Invasion Chamber (BD Biosciences), primed according to the manufacturer's directions. Cell suspensions containing approximately 5×10³ cells in serum-free medium containing various concentrations of each of the candidate peptides are placed in each upper chamber of the Matrigel™ plate. Serum-free medium without any supplement is used as control. A chemoattractant (for example, NIH-3T3 fibroblasts conditioned medium) is placed in each lower chamber. After incubation of 24 h at 37° C. and 5% CO₂, the filter at the bottom of each cell is excised following the manufacturer's instructions. Cells that did not penetrate the filter are removed with cotton swabs and cells that migrated to the lower surface of the filter are stained [for example, with Hema-Diff™ Rapid Differential Stain (StatLab, Tx)] and analyzed under the microscope (Leica DMi 3000M, Germany) at ×10 magnification.

It will be appreciated that the disruption of actin assembly and disassembly can affect cell motility, development and growth. Indeed, actin-binding RNASET2 peptides have demonstrated significant anti-angiogenic and anti-tumor properties in both in-vitro (e.g. HUVEC Matrigel angiogenesis assay) and in-vivo (e.g. CAM assay or mouse xenograft assay) assays (see Examples section below). Thus, in some embodiments of any aspects of the invention, the isolated T2 RNase peptide of the invention has a biological activity other than actin binding. In some embodiments of the invention, such a biological activity can include inhibition of angiogenesis, prevention, inhibition or reversal of cell motility and/or metastatic transformation and spread, clonogenicity, differentiation and/or development of abnormally proliferating cells, tumor growth and oncogenic transformation.

Inhibition of angiogenesis by the isolated peptide of the invention can be assessed in in-vitro assays, ex-vivo assays or in-vivo. Suitable in-vitro angiogenesis assays include endothelial cell culture (HUVEC, microvascular endothelial culture, etc), endothelial cell migration assays (e.g. Boyden chamber, wound healing assay), endothelial cell differentiation assays (e.g. tubule formation), and endothelial cell co-culture with mural cells or fibroblasts. Ex-vivo assays for angiogenesis include monitoring of microvessel outgrowth in organ explants (endothelial organs, such as aortal rings) culture. Various in-vivo assays of angiogenesis are available, such as the chick chorio-allantoic membrane assay, zebrafish embryo assay, sponge or polymer implantation assay, corneal angiogenesis assay and dorsal skin-fold (airsac) assay. The isolated peptide of the invention can also be used for inhibition of tumor angiogenesis, thus in-vivo assays of tumor angiogenesis are also informative. Detailed explanation of many of the commonly used angiogenesis assays is provided in Staton, et al (Int J Exp Path, 2009).

Anti-cancer and anti-angiogenic activity of the isolated peptide can be examined as follows:

Colony-Formation Assay:

Exemplary colony-forming assays can be performed as follows: Briefly, cancer cells [e.g. Human colon (1-IT-29) cancer cells] are grown in 50-ml flasks (10⁵ cells per flask). The medium contains 7 ml DMEM supplemented with 10% fetal calf serum (FCS), 1% glutamine, and 1% antibiotic-antimycotic solution, and added candidate T2 RNase peptides. The cells are incubated at 37° C. in a humidified atmosphere containing 5% CO₂. After 48 h, cells (approx 10³ cells/well) are seeded in 96-well plates in 200 μl medium, in the presence or absence of predetermined concentrations of the candidate T2 RNases or T2 RNase peptides. After 5 days, the cells are fixed in 4% formaldehyde and stained with methylene blue. The number of colonies is then counted to determine inhibition of colony formation and growth.

Human Umbilical Vein Endothelial Cell (HUVEC) Angiogenesis Assay:

HUVEC (CC-2519)(fresh or commercially provided) are maintained in M199 rrrediurn supplemented with 20% FCS, 1% glutamine, 1% antibiotic-antimycotic solution, ECGF, and heparin, plated in a 96-well plate (14×10³ cells/well) previously coated with growth factor-depleted Matrigel™ (BD Biosciences), in M199 medium containing 5% FCS and 0.005% ECGF. The wells are supplemented with candidate T2 RNase peptides or PBS, and angiogenic growth factors (1 μg/ml angiogenin, bFGF or VEGF) to a final volume of 120 pl. Plates are incubated (overnight), photographed and the extent of tube formation (angiogenesis) assessed.

Chicken Chorio-Allantoic Membrane (CAM) Angiogenesis Assay:

In this assay, in-vivo angiogenesis is assessed on live chick embryos grown in petri dishes. Fertilized eggs are cracked into a petri dish after a few (e.g. 3) days incubation at appropriate temperature (e.g. 37° C.) and humidity. In the following days, the embryos developed a vascular network, which can then easily be exposed to any treatment or combination of treatments, for example, via delivery onto absorbent disks placed on/about the developing vascular regions. Angiogenesis is scored by periodically (e.g. 1×/day, 2×/day, 1×/2 days, 1×/4 days, etc) counting the number of blood vessels around the disk. Comparison to control embryos reveals effect (or lack thereof) on in-vivo angiogenesis.

Cellular motility assays suitable for use with the T2 RNase peptides of the invention include, but are not limited to the Phagokinetic assay (gold particles are phagocytized by the cell) and the Transwell Migration or Boyden Chamber assays.

Metastatic transformation of tumors can be assessed, for example, by the soft agar transformation test, assessing growth of cells suspended in agar. In-vivo, transition from pre-cancerous to cancerous states can be monitored, for example, as in the appearance of aberrant crypt foci in induced colon cancer models. Xenografts also provide an opportunity to assess tumor growth, tumor angiogenesis and metastatic transformation.

An exemplary xenograft assay is as follows:

Xenograft Model:

Cancer cells (e.g. HT-29 cancer cells or A375SM supermetastatic cells) are injected subcutaneously into the left hip of athymic mice. When the tumors are palpable (10-13 days after cancer cell injection), the candidate T2 RNase peptides, or PBS alone are injected into the tail vein, (IV) in a predetermined regimen (for example, every other day (three times a week), totaling 12-15 injections altogether). During the experiment, the tumors are measured at regular intervals (for example, twice a week, three times or more a week). After 5, 7, 10, 14, 21, 25 or 30 or more days, mice are sacrificed and the tumors or the area of injection excised for size measurements and histopathological examination. Tumor volume is calculated using the equation (length×width)/2. Angiogenic and apoptotic status of the tumors can be assessed in the median tumor cross-sections, for example, by Hematoxylin and Eosin stain, anti-CD31 immunostaining or as described in Matsuzaki et al (2007, Calcif Tissue Int. 80: 391-399). Using image analysis software (Image J; NIH, Bethesda, Md.), a binary image is created using a threshold value midway between background (white) and blood vessels (black). The number and size of all black objects (blood vessels) greater than 10 pixels in size is determined using the particle analysis function of Image J. Vessel number, total vessel area and the relative area (the ratio between total blood-vessel area and tumor-section area) are determined from these data. From each tumor section, 2-4 different field areas are determined. Means are compared by using an analysis of variance and analyzed statistically for significance. Apoptotic status can be measured by TUNEL or FragEL™ TdT staining.

The isolated peptide of the invention effectively binds actin, which is the basic and most abundant component of the cellular cytoskeleton. Disruption of actin assembly and disassembly affects cell motility, development, growth, proliferation and reproduction. Thus, in some embodiments, the peptides of the present invention can be used for treating conditions, syndromes or diseases characterized by abnormal accumulation of cells. Further, the T2 RNase peptides of the present invention, and compositions comprising such, may be used as therapeutic agents for controlling cellular disorders related to motility, and diseases or conditions characterized by abnormal accumulation of cells including, but not limited to, cancer (e.g. tumor angiogenesis and metastasis), disorders of immune regulation, neurodegenerative and inflammatory diseases.

Some inflammatory diseases or conditions are characterized by abnormal angiogenesis, for example, pathological situations such as atherosclerosis, diabetes, and arthritis. The inflammatory response increases capillary permeability and induces endothelial activation, which, when persistent, results in capillary sprouting. Thus, the T2 RNase peptides of the present invention can be used as therapeutic agents for treating or preventing disorders or diseases of inflammation-associated angiogenesis, such as atherosclerosis, diabetes, and arthritis.

The isolated T2 RNase peptides of the invention can be used to prepare a medicament according to the present invention by means of conventional mixing, dissolving, granulating, dragee-making, levigating, emulsifying, encapsulating, entrapping or lyophilizing processes with the addition of the appropriate pharmaceutically acceptable carriers and/or excipients or alternatively it can be linked to appropriate delivery vehicles as described hereinabove.

Thus, according to one embodiment of the present invention there is provided a method of inhibiting angiogenesis in a subject in need thereof. The method is effected by providing an isolated peptide of the invention having actin binding activity.

The compositions and methods of the present invention can be used for inhibiting actin filament assembly and disassembly in a cell or a tissue, affected by providing to the cell or tissue an isolated peptide of the invention having actin binding activity, for example, SEQ ID NO: 57, 81, 82, 83, 130, 133, 62, 131 or SEQ ID NO: 132. In some embodiments of the present invention the compositions and methods comprise an isolated RNASET2 peptide having the amino acid sequence as set forth in SEQ ID NO: 57.

Thus, the peptides present invention can be used for treating conditions, syndromes or diseases characterized by abnormally proliferating cells, such as cancerous or other cells, such as, but not limited to, a malignant or non-malignant cancer including biliary tract cancer; brain cancer; breast cancer; cervical cancer; choriocarcinoma; endometrial cancer; esophageal cancer; gastric cancer; intraepithelial neoplasms; lymphomas; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas, papilloma, blastoglioma, Kaposi's sarcoma, squamous cell carcinoma, astrocytoma, head cancer, neck cancer, bladder cancer, colorectal cancer, thyroid cancer, pancreatic cancer, gastric cancer, hepatocellular carcinoma, leukemia, lymphoma, Hodgkin's disease, Burkitt's disease, arthritis, rheumatoid arthritis, diabetic retinopathy, angiogenesis, restenosis, in-stent restenosis, vascular graft restenosis, proliferative vitreoretinopathy, chronic inflammatory proliferative disease, dermatofibroma and psoriasis.

As used herein the terms “cancer” or “tumor” are clinically descriptive terms which encompass a myriad of diseases characterized by cells that exhibit abnormal cellular proliferation. The term “tumor”, when applied to tissue, generally refers to any abnormal tissue growth, characterized in excessive and abnormal cellular proliferation. A tumor may be “benign” and unable to spread from its original focus, or “malignant” or “metastatic” and capable of spreading beyond its anatomical site to other areas throughout the host body. The tumor may be a “primary” tumor, residing in the organ in which it has developed, and which is not a metastatic growth, or it may be a metastatic tumor, developing in an organ other than that of the primary tumor. A tumor may be a “solid tumor”, or a “fluid filled, cystic” tumor. The term “cancer” is an older term which is generally used to describe a malignant tumor or the disease state arising therefrom. Alternatively, the art refers to an abnormal growth as a neoplasm, and to a malignant abnormal growth as a malignant neoplasm. The term “tumor” is also refers to tissue “nodes” or tissue “masses”.

The isolated T2 RNase peptides of the present invention can be used in the preventive treatment of a subject at risk of having a cancer. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is exposed to the isolated T2 RNase peptides of the present invention, the subject may be able to prevent any cancer that does form from becoming metastatic.

The isolated T2 RNase peptides of the present invention are also useful for treating and/or preventing disorders associated with inflammation in a subject. Immune or hematopoietic cells exposed to isolated T2 RNase peptides having an actin binding activity would have a reduced ability to migrate. Thus, in some embodiments isolated T2 RNase peptides having actin binding activity of the invention are useful for preventing inflammation associated with immune cell migration and for treating and preventing inflammatory disorders and ischemic diseases.

Inflammatory disorders and ischemic diseases are characterized by inflammation associated with neutrophil migration to local tissue regions that have been damaged or have otherwise induced neutrophil migration and activation. While not intending to be bound by any particular theory, it is believed that excessive accumulation of neutrophils resulting from neutrophil migration to the site of injury, causes the release toxic factors that damage surrounding tissue. When the inflammatory disease is an acute stroke a tissue which is often damaged by neutrophil stimulation is the brain. As the active neutrophils accumulate in the brain an infarct develops.

An “inflammatory disease or condition” as used herein refers to any condition characterized by local inflammation at a site of injury or infection and includes autoimmune diseases, certain forms of infectious inflammatory states, undesirable neutrophil activity characteristic of organ transplants or other implants and virtually any other condition characterized by unwanted neutrophil accumulation at a local tissue site. These conditions include but are not limited to meningitis, cerebral edema, arthritis, nephritis, adult respiratory distress syndrome, pancreatitis, myositis, neuritis, connective tissue diseases, phlebitis, arteritis, vasculitis, allergy, anaphylaxis, ehrlichiosis, gout, organ transplants and/or ulcerative colitis.

An “ischemic disease or condition” as used herein refers to a condition characterized by local inflammation resulting from an interruption in the blood supply to a tissue due to a blockage or hemorrhage of the blood vessel responsible for supplying blood to the tissue such as is seen for myocardial or cerebral infarction. A cerebral ischemic attack or cerebral ischemia is a form of ischemic condition in which the blood supply to the brain is blocked. This interruption in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue.

In some aspects of the invention the isolated T2 RNase peptides of the present invention are provided in an effective amount to prevent migration of a tumor cell across a barrier. The invasion and metastasis of cancer is a complex process which involves changes in cell adhesion properties which allow a transformed cell to invade and migrate through the extracellular matrix (ECM) and acquire anchorage-independent growth properties (Liotta, L. A., et al., Cell 1991 64:327-336). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

In yet another embodiment, the isolated T2 RNase peptides of the present invention can be used to assay cells for sensitivity to inhibition of cellular motility, for example, in testing their ability to cross a barrier. In some embodiments the tumor cells are prevented from crossing a barrier. The barrier for the tumor cells may be an artificial barrier in vitro or a natural barrier in vivo. In vitro barriers include but are not limited to extracellular matrix-coated membranes, such as Matrigel™. Thus, isolated T2 RNase peptides can be provided to cells which can then be tested for their ability to inhibit tumor cell invasion in a Matrigel™ invasion assay system. Other in vitro and in vivo assays for metastasis have been described in the prior art, see, e.g., U.S. Pat. No. 5,935,850, which is incorporated herein by reference. An in vivo barrier refers to a cellular barrier present in the body of a subject.

The isolated T2 RNase peptides according to one aspect of the present invention can be administered to an organism, such as a human being or any other mammal, per se, or in a pharmaceutical composition where the peptide or peptides are mixed with suitable carriers or excipients.

As used herein a “pharmaceutical composition” or “medicament” refers to a preparation of one or more of the isolated T2 RNase peptides as described herein, with other chemical components such as physiologically suitable carriers and excipients. The purpose of a pharmaceutical composition is to facilitate administration of a compound to an organism.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Pharmaceutical compositions may also include one or more additional active ingredients, such as, but not limited to, anti inflammatory agents, antimicrobial agents, anesthetics, cancer therapeutic agents and the like in addition to the main active ingredient. A detailed description of commonly used additional agents suitable for use with the compositions of the present invention is presented hereinbelow.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which can be used pharmaceutically. Proper formulation is dependent upon the route of administration chosen.

Significant therapeutic effects of ribonucleases of the T2 family have been revealed using a broad variety of means of administration, in diverse models of abnormal cell proliferation and accumulation, angiogenesis, metastatic proliferation and tumor growth (see WO 2006/035439 and WO 2010/049933, fully incorporated herein by reference). Intraperitoneal administration of T2 RNase was found effective in suppressing tumor growth and development in subcutaneous tumors in nude mice and intraperitoneal tumors. Intravenous administration, providing even more rapid systemic uptake of T2 RNase, was also found effective in suppressing and treating subcutaneous xenografts and remote (lung) metastatic spread of intravenous tumors. Direct administration of, and preincubation of cells with T2 RNase has been found effective in preventing tumor growth in breast carcinoma, colon carcinoma, melanoma in-vivo, angiogenic factor induced angiogenesis and microvessel density and cell tube formation in both plant and human HUVE cells in-vitro. Oral administration of T2 RNase, in the form of microcapsules, has been found effective in reducing tumor growth, clonogenicity, tumor size, tumor vascularization and the number of aberrant crypt foci when administered early in colon tumor (DMH model) induction. Similar oral administration of T2 RNase to animals harboring already well developed tumors reduced the degree of vascularization and malignancy of colon cancer tumors in rats, despite exposure of the RNase to digestive processes and low doses presumed delivered intraintestinally. It will be appreciated that encapsulation methods providing effective intestinal release of compositions are well known in the art, and use of such is expected to increase the effectiveness of oral administration of isolated T2 RNase peptides in cases of already established tumors.

The T2 RNase peptides of the invention can be used to directly affect tumor growth, angiogenesis and metastatic transformation of tissues, in particular, tissues having cells exhibiting abnormal and/or aberrant proliferation. Thus, according to some embodiments of the invention, there is provided a method of inhibiting angiogenesis, tumor growth and/or metastatic transformation in a tissue (e.g. a tumor, cancerous tissue and the like), comprising contacting the tissue with a therapeutically effective amount of the isolated peptide or compositions of the invention.

Thus, to effect administration the pharmaceutical composition of the present invention includes a suitable pharmaceutical carrier and an effective amount of an isolated T2 RNase peptide having actin binding activity of the invention, and is administered, for example, topically, intraocularly, parenterally, orally, intranasally, intravenously, intramuscularly, intraperitoneally, subcutaneously or by any other effective means via methods well known in the art.

For intravenous, intraperitoneal, intramuscular or subcutaneous injection, the isolated T2 RNase peptides may be formulated in aqueous solutions, for example, in physiologically compatible buffers such as Hank's solution, Ringer's solution, or physiological saline buffer. For example, a physiologically appropriate solution containing an effective amount of an isolated T2 RNase peptide or peptides of the invention can be administered systemically into the blood circulation to treat a cancer or tumor which cannot be directly reached or anatomically isolated. A physiologically appropriate solution containing an effective amount of an isolated T2 RNase peptide or peptides may be directly injected into a target cancer or tumor tissue by a needle or catheter in amounts effective to treat the tumor cells of the target tissue.

For transmucosal administration, penetrants appropriate to the barrier to be permeated are used in the formulation. Such penetrants are generally known in the art.

For oral administration, the pharmaceutical composition of the present invention can be formulated readily by combining an isolated T2 RNase peptide or peptides of the invention with pharmaceutically acceptable carriers well known in the art. Such carriers enable the isolated T2 RNase peptide or peptides to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Additional pharmaceutical compositions, which can be used orally, include push-fit capsules made of gelatin as well as soft, sealed capsules made of gelatin and a plasticizer, such as glycerol or sorbitol. The push-fit capsules may contain the isolated T2 RNase peptide or peptides of the invention in admixture with filler such as lactose, binders such as starches, lubricants such as talc or magnesium stearate and, optionally, stabilizers. In soft capsules, the isolated T2 RNase peptide or peptides of the invention may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Oral delivery of the pharmaceutical composition of the present invention may not be successful due to the pH and enzyme degradation present in the gastrointestinal tract. Pharmaceutical compositions can be formulated to avoid undesirable circumstances. For example, enteric coating can be applied to oral solid formulation. Substances with acidic-resistant properties such as cellulose acetate phthalate (CAP), hydroxypropyl methycellulose phthalate (HPMCP) and acrylic resins are most commonly used for coating tablets or granules for micro encapsulation. Wet granulation can be used to prepare the enteric-coated granules to avoid reactions between the active ingredient and the coating (Lin, S. Y. and Kawashima, Y. 1987, Pharmaceutical Res. 4:70-74). A solvent evaporation method can also be used. The solvent evaporation method was used to encapsulate insulin administered to diabetic rats to maintain blood glucose concentration (Lin, S. Y. et al., 1986, Biomater, Medicine Device, Artificial organ 13:187-201 and Lin, S. Y. et al., 1988, Biochemical Artificial Cells Artificial Organ 16:815-828). It was also used to encapsulate biological materials of high molecular weight such as vial antigen and concanavalin A (Maharaj, I. Et al. 1984, J. Pharmac. Sci. 73:39-42).

For buccal administration, in one embodiment, the pharmaceutical composition of the present invention may take the form of tablets or lozenges formulated in conventional manner.

For administration by inhalation, the isolated T2 RNase peptide or peptides of the invention, or polynucleotides encoding same, for use according to one embodiment of the present invention is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of an isolated T2 RNase peptide or peptides, or polynucleotides encoding the peptides of the invention and a suitable powder base such as lactose or starch.

According to another embodiment, the pharmaceutical composition of the present invention may also be formulated for parenteral administration, e.g., by bolus injection or continuous infusion. A composition for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the isolated T2 RNase peptide or peptides of the invention may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the isolated T2 RNase peptide or peptides of the invention to allow for the preparation of highly concentrated solutions.

Alternatively, an isolated T2 RNase peptide or peptides of the invention may be in a powder form for constitution with a suitable vehicle, e.g., sterile, pyrogen-free water, before use.

The pharmaceutical composition of the present invention may also be formulated in rectal compositions such as suppositories or retention enemas, using, e.g., conventional suppository bases such as cocoa butter or other glycerides.

In addition, a cancer or tumor present in a body cavity, such as in the eye, gastrointestinal tract, genitourinary tract (e.g., the urinary bladder), pulmonary and bronchial system and the like, can receive a physiologically appropriate composition (e.g., a solution such as a saline or phosphate buffer, a suspension, or an emulsion, which is sterile) containing an effective amount of an isolated T2 RNase peptide or peptides of the invention via direct injection with a needle or via a catheter or other delivery tube placed into the cancer or tumor afflicted hollow organ. Any effective imaging device such as X-ray, sonogram, or fiber optic visualization system may be used to locate the target tissue and guide the needle or catheter tube in proximity thereto.

Yet further, the peptides or compositions of the present invention can also be delivered as a dry, non-aqueous formulation, comprised within non-aqueous, water insoluble polymer matrix, for implantation, for example, into or in proximity with the target tissues or cells. Such a method of administration is particularly suited for delivery of compositions comprising peptides formulated with liposome delivery vehicles, as detailed, for example, in U.S. Patent Application Publication No. 20110244029. Delivery vehicles for local administration can also include lipid-protein complexes as detailed in U.S. Patent Application Publication Nos. 20110117197, 20120119756, 20120294944, 20140271861 and 20140335165 and can also be of the kind of controlled/slow release type.

The pharmaceutical composition of the present invention can also be delivered by osmotic micro pumps. The osmotic micro pumps are implanted into one of the body cavities and the drug is constantly released onto the tissue to be treated. This method is particularly advantageous when an immune response to the pharmaceutical composition is experienced. This method has been employed for ONCONASE (Vasandani V. M., et al., 1996, Cancer Res. 15; 56(18):4180-6).

Alternatively and according to yet another embodiment of the present invention, the pharmaceutically acceptable carrier includes a delivery vehicle capable of delivering an isolated T2 RNase peptide or peptides of the invention to cells or tissue.

Numerous delivery vehicles and methods are known in the art for targeting proteins or nucleic acids into or onto tumors or cancer cells. For example, liposomes are artificial membrane vesicles that are available to deliver proteins or nucleic acids into target cells (Newton, A. C. and Huestis, W. H., Biochemistry, 1988, 27:4655-4659; Tanswell, A. K. et al., 1990, Biochmica et Biophysica Acta, 1044:269-274; and Ceccoll, J. et al., Journal of Investigative Dermatology, 1989, 93:190-194). Thus, a T2-RNase or T2 RNase peptide or a polynucleotide encoding same can be encapsulated at high efficiency with liposome vesicles and delivered into mammalian cells. In addition, the isolated T2 RNase peptide or peptides of the invention or nucleic acid can also be delivered to target tumor or cancer cells via micelles as described in, for example, U.S. Pat. No. 5,925,628 to Lee, which is incorporated herein by reference.

Liposome- or micelle-encapsulated isolated T2 RNase peptide or peptides of the invention may be administered topically, intraocularly, parenterally, intranasally, intratracheally, intrabronchially, intramuscularly, subcutaneously or by any other effective means at a dose efficacious to treat the abnormally proliferating cells of the target tissue. The liposomes may be administered in any physiologically appropriate composition containing an effective amount of encapsulated isolated T2 RNase peptide or peptides of the invention.

Alternatively and according to still another embodiment of the present invention the delivery vehicle can be, but it is not limited to, an antibody or a ligand capable of binding a specific cell surface receptor or marker. An antibody or ligand can be directly linked to an isolated T2 RNase peptide or peptides of the invention via a suitable linker, or alternatively such an antibody or ligand can be provided on the surface of a liposome encapsulating an isolated T2 RNase peptide or peptides of the invention.

For example, an isolated T2 RNase peptide or peptides of the invention, or polynucleotides encoding same can be fused with specific membranal protein antibodies or ligands for targeting to specific tissues or cells as previously described in the art. It will be appreciated in this respect that fusion of RNase A of the ribonuclease A superfamily with antibodies to the transferrin receptor or to the T cell antigen CD5 lead to inhibition of protein synthesis in tumor cells carrying a specific receptor for each of the above toxins (Rybak, M. et al., 1991, J. Biol. Chem. 266:21202-21207 and Newton D L, et al., 1997, Protein Eng. 10(4):463-70).

Pharmaceutical compositions suitable for use in context of the present invention include compositions wherein the active ingredients are contained in an amount effective to achieve the intended purpose. More specifically, a therapeutically effective amount means an amount of the active ingredients effective to prevent, alleviate or ameliorate symptoms of disease or prolong the survival of the subject being treated.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Determination of a therapeutically effective amount is well within the capability of those skilled in the art, especially in light of the detailed disclosure provided herein.

Toxicity and therapeutic efficacy of the active ingredients described herein can be determined by standard pharmaceutical procedures in cell cultures or experimental animals, e.g., by determining the IC₅₀ and the LD₅₀ (lethal dose causing death in 50% of the tested animals) for a subject active ingredient. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to yet another aspect of the present invention there are provided methods of enhancing therapeutic treatment of a cancer. The methods are effected by administering to a subject in need thereof, in combination with the therapeutic treatment, an isolated T2 RNase peptide or peptides of the invention. It will be appreciated that such synergistic activity of the isolated T2 RNase peptide or peptides of the invention with additional therapeutic methods or compositions has the potential to significantly reduce the effective clinical doses of such treatments, thereby reducing the often devastating negative side effects and high cost of the treatment.

Therapeutic regimen for treatment of cancer suitable for combination with the isolated T2 RNase peptide or peptides of the present invention or polynucleotide encoding same include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

Anti-cancer drugs that can be co-administered with the peptides and compounds of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Anti-inflammatory drugs that can be administered in combination with the T2 RNase peptide/s or polynucleotide encoding same of the present invention include but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

In yet another embodiment of the present invention, gene therapy with isolated T2 RNase peptide or peptides of the invention is envisaged. According to this aspect of the present invention a polynucleotide encoding an isolated T2 RNase peptide or peptides of the invention is introduced into a mammalian cell along with a pharmaceutically acceptable carrier, which introduction results in a genetic modification of this cell, enabling the expression of the isolated T2 RNase peptide or peptides of the invention therein.

Acquati et al have shown that transfection of RNase 6PL cDNA into HEY4 and SG10G ovarian tumor cell lines suppresses tumorigenicity in nude mice (Aquati et al. Oncogene. 2001 22; 20(8):980-8), thus demonstrating the feasibility of such genetic modification with T2 RNase.

As used herein in the specification and in the claims section below, the term “genetic modification” refers to a process of inserting nucleic acids into cells. The insertion may, for example, be effected by viral infection, injection, transfection, particle bombardment or any other means effective in introducing nucleic acids into cells, some of which are further detailed herein below. Following the genetic modification the nucleic acid is either integrated in all or part, to the cell's genome (DNA), or remains external to the cell's genome, thereby providing stably modified or transiently modified cells.

As used herein the phrases “gene therapy” or “genetic therapy” are used interchangeably and refer to a method of therapy in which a stable or transient genetic modification of a proliferative cell(s) such as a cancer cell, leads to the inhibition of proliferation of this cell. Any polynucleotides encoding isolated T2 RNase peptide or peptides of the invention, for example SEQ ID NOs: 85-88 and the like can be employed according to the present invention as a polynucleotide encoding isolated T2 RNase peptide or peptides of the invention. In addition, polynucleotides homologous to any of SEQ ID NOs: 85-88 can also be employed as a polynucleotide encoding a isolated T2 RNase peptide or peptides of the invention, provided that the protein encoded thereby is characterized as an isolated T2 RNase peptide and exhibits the desired actin-binding and anti-angiogenic activities. Furthermore, it will be appreciated that portions, mutants, chimeras or alleles of such polynucleotides can also be employed as a polynucleotide encoding an isolated T2 RNase peptide or peptides of the invention according to one embodiment of the present invention, again, provided that such portions, mutants chimeras or alleles of such polynucleotides encode an isolated T2 RNase peptide or peptides of the invention which exhibits the desired activities.

In another embodiment, a polynucleotide according to the present invention can be fused, in frame, to any other peptide- or protein-encoding polynucleotide to encode for a fused peptide or protein using methods well known in the art. In one embodiment, the polynucleotide encoding an isolated T2 RNase peptide or peptides of the invention is fused to a polynucleotide encoding a recognition entity peptide (e.g. His-tag). In yet a further embodiment, an optional polynucleotide sequence encoding a protease cleavage site (e.g. TEV cleavage site, enterokinase cleavage site, thrombin cleavage site, etc) is inserted in between the polynucleotide encoding the isolated T2 RNase peptide or peptides of the invention and the polynucleotide encoding the recognition entity peptide, encoding an T2 RNase-cleavage site-recognition entity peptide fusion protein. The cleavage site and recognition entity sequences can be fused to the N-terminal or C-terminal region of the isolated T2 RNase peptide or peptides of the invention.

An isolated T2 RNase peptide or peptides of the invention can be fused (conjugated) to other peptides or proteins using methods well known in the art. Many methods are known in the art to conjugate or fuse (couple) molecules of different types, including proteins. These methods can be used according to the present invention to couple an isolated T2 RNase peptide or peptides of the invention to other molecules such as ligands or antibodies to thereby assist in targeting and binding of the isolated T2 RNase peptide or peptides of the invention to specific cell types. Any pair of proteins can be conjugated or fused together using any conjugation method known to one skilled in the art. The proteins can be conjugated using a 3-(2-pyridyldithio) propionic acid N-hydroxysuccinimide ester (also called N-succinimidyl 3-(2pyridyldithio) propionate) (“SDPD”) (Sigma, Cat. No. P-3415), a gluteraldehyde conjugation procedure or a carbodiimide conjugation procedure.

Expression vectors compatible with mammalian host cells for use in genetic therapy of tumor or cancer cells, include, but are not limited to, plasmids, retroviral vectors, adenovirus vectors, herpes viral vectors, and non-replicative avipox viruses, as disclosed, for example, by U.S. Pat. No. 5,174,993.

Several methods can be used to deliver the expression vector according to this aspect of the present invention to the target mammalian cell(s).

According to yet another aspect of the present invention there is provided an anti-isolated T2 RNase peptide antibody, capable of specifically binding isolated T2 RNase peptide or peptides of the invention. In some embodiments, the antibody specifically binds at least one epitope of an isolated T2 RNase peptide or peptides of the invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). In one embodiment, the anti-T2 RNase peptide antibody is a polyclonal antibody raised in rabbits against the whole isolated T2 RNase peptide or peptides of the invention.

Isolated T2 RNase peptide or peptides binding actin of the invention and compositions (e.g., pharmaceutical composition) comprising same may be used in diagnostic and therapeutic applications and as such may be included in therapeutic or diagnostic kits.

Thus, compositions and combinations of compositions of the isolated peptide of the present invention may, if desired, be presented in an article of manufacture such as a pack or dispenser device, such as an FDA approved kit, which may contain one or more unit dosage forms containing the active ingredient i.e., isolated T2 RNase peptide or peptides of the invention. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Compositions comprising a peptide of the invention formulated in a compatible pharmaceutical carrier may also be prepared, placed in an appropriate container, and labeled for treatment or prevention of an indicated condition or induction of a desired event. Suitable indica on the label may include treatment and/or prevention of cellular disorders related to motility, and diseases or conditions characterized by abnormal accumulation of cells including, but not limited to, cancer (e.g. tumor angiogenesis and metastasis), disorders of immune regulation, neurodegenerative and inflammatory diseases and the like.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

It is expected that during the life of a patent maturing from this application many relevant actin-binding T2 RNase peptides will be developed and the scope of the term “actin binding” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1, 2, 317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Actin Binding RNASET2 Peptides

Peptides:

Peptides corresponding to a variety of amino acid sequences in the region of human RNASET2 comprising helix 5 and helix 6 were synthesized and assayed for actin binding in an ELISA actin binding assay.

ELISA Actin Binding Assay:

Solid-phase ELISA for RNASET2 peptide binding to immobilized actin was performed as modified from Mejean 1987 (Biochem. J. 244: 571-577) and Mejean 1992 (Eur. J Biochem. 209: 555-562). All steps were conducted at 37° C. Immuno-96 Microwell™ solid plates (Thermo Fisher Scientific, MA) coated with 500 ng/well of G-Actin from rabbit muscle (Sigma-Aldrich, St Louis, Mo.) in 100 ul of 0.05 M carbonate buffer, pH 9.5 (Sigma-Aldrich, Mo), incubated for 1 h and washed with 250 ul/well Tris Buffered Saline (TBS) (Sigma-Aldrich). The wells were blocked with 3% bovine serum albumin (BSA, Sigma-Aldrich) in 200 ul/well TBS for another 1 hour and washed with 250 ul/well TBS. The candidate peptides are then added at various concentrations (for example, serial dilutions giving 500, 250 or 125 ng/well) in 100 ul/well Tris Buffered Saline (TBS), incubated for 1 hour and washed 3 times with 250 ul/well TBS containing 0.05% Tween®20 (TBST, Sigma-Aldrich). Anti-T2 RNase (rabbit anti-rhtrRNASET2, Anilab, Israel, whole serum or antiserum) was then added, incubated for 1 hour and the plates washed 3 times with 250 ul/well TBST. Labeled second antibody [peroxidase-conjugated affinity pure goat-anti rabbit IgG (Jackson, Pa.)] was added at a dilution of 1:10:000 in 100 ul/well TBS, incubated for 1 hour and the plates washed twice with 250 ul/well TBST. After an additional wash with 250 ul/well TBS, 1-Step Ultra TMB-ELISA (TMB-3,3′,5,5′ Tetramethylbenzidine) (Thermo-Scientific) was added (100 ul/well) and after 15-25 min of incubation, the reaction was read at A650 using Infinite F50-TECAN ELISA READER (TECAN, Austria). Each assay was performed in triplicates.

Results: Actin Binding of Helix 5-Containing Peptides

Peptides representing serial truncations of the amino acid sequence corresponding to helix 5-linker-helix 6 region of human RNASET2 were assayed, in order to determine whether this region includes an actin-binding motif. As has been repeatedly shown, the truncated RNASET2 polypeptide trT2-49 (SEQ ID NO: 84), representing amino acids 49-232 of full length, native RNASET2, effectively binds actin. Within the sequence of the truncated trT2-49, a sub-sequence representing amino acids 120-141 of native human RNASET2 polypeptide (SEQ ID NO: 58) can be discerned, which includes the sequence proposed (Kumar et al. 2012) to be responsible for RNASET2's actin binding properties. As shown in FIG. 2, SEQ ID NO: 58 was unable to bind actin in the ELISA actin binding assay. A smaller peptide, SEQ ID NO: 59, representing a C-terminal portion of SEQ ID NO: 58, which was also identified by Kumar et al (J Mol. Mod. 2013) and Gundampati et al (J Mol Mod 2012) as critical for actin binding of RNASET2, also lacked any discernible actin binding activity on ELISA.

Peptides representing portions of the region of RNASET2 including helix 5, and helix 6, however, showed a range of actin binding activity on the ELISA assay. Both a large peptide representing amino acids 103-159 of RNASET2 (SEQ ID NO: 60) and a smaller portion thereof representing amino acids 103-141 of RNASET2 (SEQ ID NO: 63) showed strong and moderate actin binding, respectively. A yet shorter 26-amino acid peptide representing amino acids 108-133 of RNASET2 (SEQ ID NO: 57) also showed strong actin binding on ELISA. ELISA actin binding assays with peptides representing serial truncations of both the C-terminal and N-terminal portions of the region representing amino acids 103-133 of RNASET2 indicated that peptide structures conferring actin binding activity of RNASET2 most probably reside within the region corresponding to helix 5 of RNASET2, and portions of the region corresponding to helix 6, when separated by a peptide or amino acid linker (see SEQ ID NOs. 57, 81, 82 and 83).

### Example H

### Actin Binding RNASET2 Peptides II

Materials and Methods

trT2-49m Polypeptide

trT2-49m polypeptide (SEQ ID NO: 129) was prepared by cloning into a pUC57 vector (Genscript Corporation, Piscataway, N.J., USA), transformation into competent E. coli DH5α, selection by ampicillin, growth and harvested for plasmid DNA preparation and sequencing. Protein expression was induced by 1 mM isopropyl β-D-thiogalactopyranoside (IPTG) (Dushefa, Haarlem, The Netherlands), the cells harvested by centrifugation and lysed with lysis buffer containing 20 mM phosphate buffer, 8 M urea, 0.1 NaCl, 1 mM EDTA (pH 8) and 2 mg/ml complete protease inhibitor (Roche Diagnostics, Mannheim, Germany). The harvested cells were then stirred for 2 h at 4° C., the lysates centrifuged at 14,000 g for 30 min at room temperature and the supernatant filtered (Whatman® FP30/0.2 μm, cellulose acetate filter). trT2-49m protein was purified from the filtrated bacterial lysate on a 1 ml HisTrap Ni-Sepharose affinity column (GE-Healthcare Bio Sciences AB, Uppsala, Sweden), eluted with an imidazol gradient (5-500 mM), prepared in equilibration buffer containing 20 mM sodium phosphate (pH 8.0), 1 M NaCl, 8 M urea and 5 mM β-mercaptoethanol using GE-Healthcare's ACTAprime plus FPLC system (GE-Healthcare Bio Sciences AB). The fractions collected from the peak were analyzed by 12.5% SDS-PAGE followed by Coomassie R250 staining. Protein containing fractions were pooled for refolding by dialysis against 20 mM Tris solution (pH 12.0). Dialysis solution was exchanged once per hour for four hours and then left overnight at room temperature. The same procedure was then used with 20 mM Tris solution (pH 10) and finally with 20 mM ammonium bicarbonate buffer (pH 8). The refolded protein was lyophilized and kept at 4° C.

Peptide Library

A peptide library containing 29 peptides based on ACTIBIND (A. niger T2 RNase) sequence and on human RNASET2 structural analysis was ordered from Genemed Synthesis Inc. (Tx USA, by HyLabs, Israel) or from GenScript Corporation (Piscataway, N.J.).

Actin Binding Solid Phase Assay

96-well plates (MaxiSorp® flat-bottom 96-well plate, Fisher Scientific Inc., Fair Lawn, N.J.) were coated with 500 ng actin in 100 μl carbonate-bicarbonate buffer (pH 9.5) for 1 h at 37° C. The plate was washed once with TBS and then blocked with 3% BSA in 200 μl TBS buffer at 37° C., for 1 h. Wells were then washed once with 250 μl TBS. trT2-49, trT2-49m or each of the 29 peptides were added at 1:2 dilutions in 100 μl TBS, starting from 500 ng/well, incubated for 1 h at 37° C. and then washed three times with TBS containing 0.1% Tween-20 (TBST). Each well was then treated with 100 μl rabbit anti-trT2-49 diluted 1:500 in TBS and incubated for 1 h at 37° C. The wells were washed three times with TBST and then incubated with 100 μl goat anti-rabbit IgG-HRP (Jackson ImmunoResearch, West Grove, Pa.) diluted 1:10,000 in TBS, for 1 h at 37° C. Wells were then washed twice with TBST, and once with TBS before 100 μl substrate (1-step Ultra TMB-ELISA, Pierce, Fisher Scientific Inc.) were added. Absorbance at 655 nm was measured 10 min thereafter using a Power Wave 200 Microplate Scanning Spectrophotometer (Bio-Tek Instrument, Winooski, Vt.). Affinity was evaluated by double reciprocal plotting.

Surface Plasmon Resonance (SPR) of Actin Binding

SPR was performed on a BIAcore 3000 instrument (BIAcore, Uppsala, Sweden). Actin was diluted in 100 mM CH3COONa pH 4.6 to a final concentration of 20 μg/ml (200 μl total) resulting in ˜1 ng immobilized actin on a CM5 BIAcore sensor chip (GE Healthcare Bio-Sciences AB) using the standard BIAcore amine coupling chemistry protocol [38]. The CM5 chip allows four separate flow cells to be operated in the BIAcore 3000 instrument used in these experiments. Coupling is achieved by activating the surface of the sensor chips with equal volumes of 50 mM N-hydroxysuccimide (NHS)/200 mM Nethyl-N-(3-diethylaminopropyl)-carboiimide (EDC) (BIAcore, AB, Uppsala, Sweden) to form activated carboxyl groups. An actin solution (15 μg/ml) was run over the chip for 5 min at a rate of 10 μl/min, allowing the amine groups on the protein to react with the activated esters. In the final stage of immobilization, the surface was blocked by 1M ethanol amine, pH 8.0. The binding assay was performed by injecting the hRNASET2, trT2-49, trT2-49m or peptides solutions in running buffer (HBS—10 mM Hepes, 150 mM Sodium Chloride, 3 mM EDTA, 0.005% Polysorbate 20) at 5 different concentrations at a flow rate of 10 μl/min at 25° C. Injections were performed simultaneously over all four channels and blank surface (a plain dextran matrix, channel 1) was used as control. The net signal was obtained by subtracting the blank signal from the signal of the immobilized surface. The association phase for protein or peptides binding to actin was followed for 4 min, and dissociation phases were monitored for 3 min. Surface regeneration between consecutive binding cycles included a 1 min injection of 1 mM NaOH. The response was monitored as a function of time (sensorgram) at 25° C. Multi-concentration data were globally fit using BIA evaluation 3.2 software.

The strength of a two molecule interaction is characterized by the equilibrium dissociation (binding) constant KD=[P][L]/[PL], where [P] is the concentration of free protein (or peptide), [L] the concentration of ligand and [PL] the concentration of the complex. At equilibrium, KD is related to the rate of complex formation (described by the association rate constant, ka) and the rate of breakdown (described by the dissociation rate constant, kd), such that KD=ka/kd. A high affinity interaction is characterized by a low KD, rapid recognition and binding by high ka, and stability of complex formation by low kd.

Ex Ovo CAM Angiogenesis Assay

Fertilized chicken eggs were incubated horizontally at 37° C. and humidity of 60-62% and cracked into Petri dishes at embryonic day 4. Incubation was continued under the same conditions. On day 8, sterile filter paper disks (5.5 mm in diameter) were layered on top of the CAM and soaked either with 5 μl PBS or 3 μg hRNASET2, trT2-49, peptide A103-Q159 (SEQ ID NO: 60) or peptide K108-K133 (SEQ ID NO: 57). Treatment was given every day for four days, then, the number of blood vessels around the treated disks was counted. Sample numbers—for hRNASET2 and trT2-49, N=3; for peptide A103-Q159 (SEQ ID NO: 60), peptide K108-K133 (SEQ ID NO: 57) and PBS, N=5.

Immunofluorescence

Cells were cultured on PBS-covered slides in 12-well plates with 0.1% pork gelatin (Sigma-Aldrich) and were incubated with peptide K108-K133 (SEQ ID NO: 57) or angiogenin. Cells were fixed with 3% Paraformaldehyde (PFA) (Merck Millipore, Dermstadt, Germany) containing 0.5% triton, washed three times with PBS and blocked with 5% donkey serum (Jackson ImmunoResearch). Rabbit anti-angiogenin (Merck Millipore) was added (1:100 dilution; prepared in 5% donkey serum) and incubated overnight at 4° C. After washing three times with TBST, the slides were incubated for 1 h with Alexa 488-conjugated anti-rabbit antibody (Invitrogen Life Technologies) and phalloidin tetramethylrhodamine B isothiocyanate-conjugated anti-rabbit antibody (Sigma-Aldrich), and then washed, and mounted with a mixture containing 30% mounting medium, 4′,6-diamidino-2-phenylindole (DAPI) (Santa Cruz Biotechnology Inc., Santa Cruz, Calif.) and 70% fluoromount (Sigma-Aldrich). The slides were viewed under a Leicactr4000 laser scanning confocal microscope.

Results

Affinity Chromatography Purification of trT2-50m

The truncated version of hRNASET2 missing the sequence of ELDLNSVLLKLGIKPSINYYQV amino acids (SEQ ID NO: 58), termed trT2-49m (SEQ ID NO: 129), was optimized for E. coli. Recombinant trT2-49m, expressed in E. coli, was purified on a HisTrap affinity column and analyzed by SDS-PAGE. A pure protein at the expected size of about 25 kDa was eluted in about 75 mM imidazol. Following protein purification and refolding, the protein was lyophilized and the yield was ˜15 mg/100 ml growth medium.

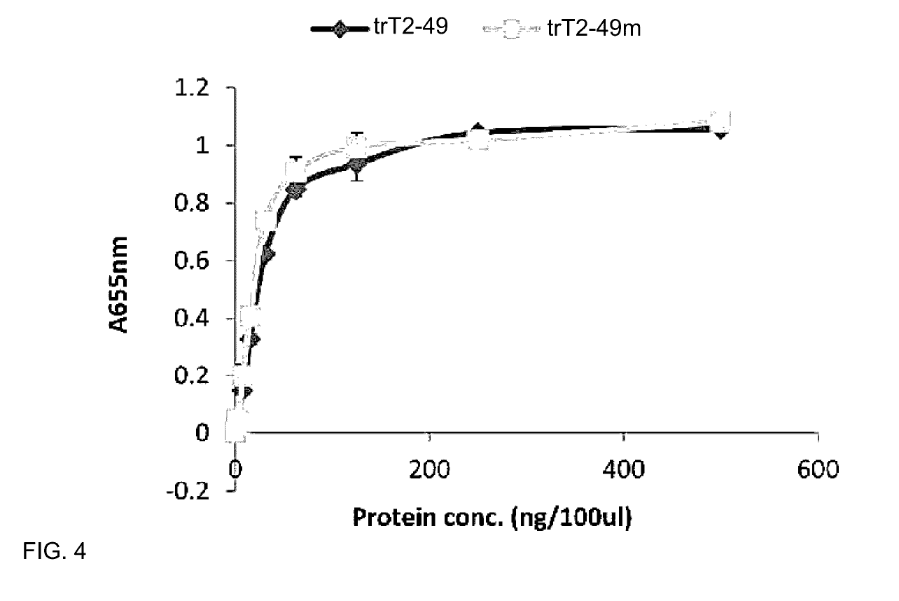

trT2-49m Binds Actin In Vitro Binding of trT2-49m to actin was analyzed by solid-phase actin binding assay. trT2-49m (SEQ ID NO: 129) bound actin in a concentration-dependent manner (FIG. 4) with a binding affinity of 34.5×10 M. This actin binding capability is similar to that of the truncated polypeptide trT2-49 (SEQ ID NO: 84). To further evaluate the strength of this interaction BIAcore analysis was performed in order to quantify and understand the kinetics of the interaction between these two molecules. Using actin coupled to a biosensor chip, the affinities of trT2-49 (SEQ ID NO: 84) and trT2-49m (SEQ ID NO: 129) were measured. Similar affinity constants were measured for both proteins. A summary of the derived constants is shown in Table 5. Furthermore, there is a linear relation between the protein concentration and the maximal (steady-state) response for hRNASET2 (SEQ ID NO: 1), trT2-49 (SEQ ID NO: 84) and trT2-49m (SEQ ID NO: 129), indicating the pseudo-first-order regime in relation to the immobilized actin. Altogether, these results indicate that the ELDLNSVLLKLGIKPSINYYQV protein sequence (SEQ ID NO: 58) is not essential for actin binding.

### Affinities Constants Measured by BIAcore Analysis.

TABLE 5
Summary table of the affinities measured by BIAcore analysis.

| | Kd | KD × 10⁻⁶ | Ka × 10³ |
|---|---|---|---|
| SEQUENCE | (1/s) | M | (1/Ms) |
| hRNASET2 (SEQ ID NO: 1) | 2.40 | 0.00346 | 1.44 |
| trT2-49 (SEQ ID NO: 84) | 7.72 | 0.0132 | 1.71 |
| trT2-49m (SEQ ID NO: 129) | 19.4 | 0.00969 | 0.50 |
| A103-Q159 (SEQ ID NO: 60) | 3.04 | 0.00322 | 1.06 |
| K108-K133 (SEQ ID NO: 57) | 14.2 | 0.0458 | 3.32 |

Human RNASET2 Peptides Bind Actin In Vitro

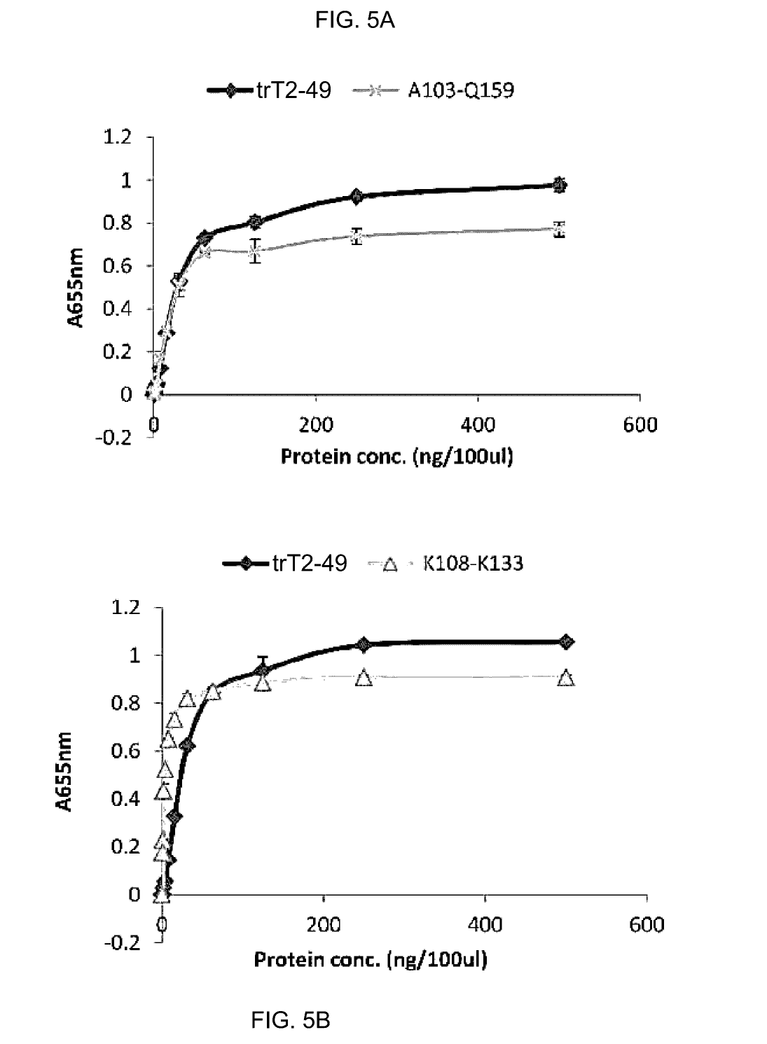

For further insight on the actin binding site within the hRNASET2 protein sequence 29 candidate peptides were tested for their ability to bind actin. The actin-binding capability of the 29 synthetic peptides was compared to the binding capability of trT2-49 (SEQ ID NO: 84) using a solid phase actin binding assay. The results obtained revealed that the peptide A103-Q159 (SEQ ID NO: 60) and the peptide K108-K133 (SEQ ID NO: 57) are the most effective actin binding peptides amongst the 29 candidate peptides (FIGS. 5A and 5B) with affinities of 68×10⁻⁹M and 10.5×10 M, respectively. Next, the interaction kinetics were tested to evaluate the interaction nature as described for trT2-49 (SEQ ID NO: 84) and trT2-49m (SEQ ID NO: 129). Similar affinity constants (same order) were measured for peptides A103-Q159 (SEQ ID NO: 60) and K108-K133 (SEQ ID NO: 57) (Table 5). In addition, there is a linear relation between the protein concentration and the maximal (steady-state) response for peptides A103-Q159 (SEQ ID NO: 60) and K108-K133 (SEQ ID NO: 57), indicating the pseudo-first-order regime in relation to the immobilized actin.

Human RNASET2 Peptides Inhibit HUVEC Tube Formation on Matrigel′

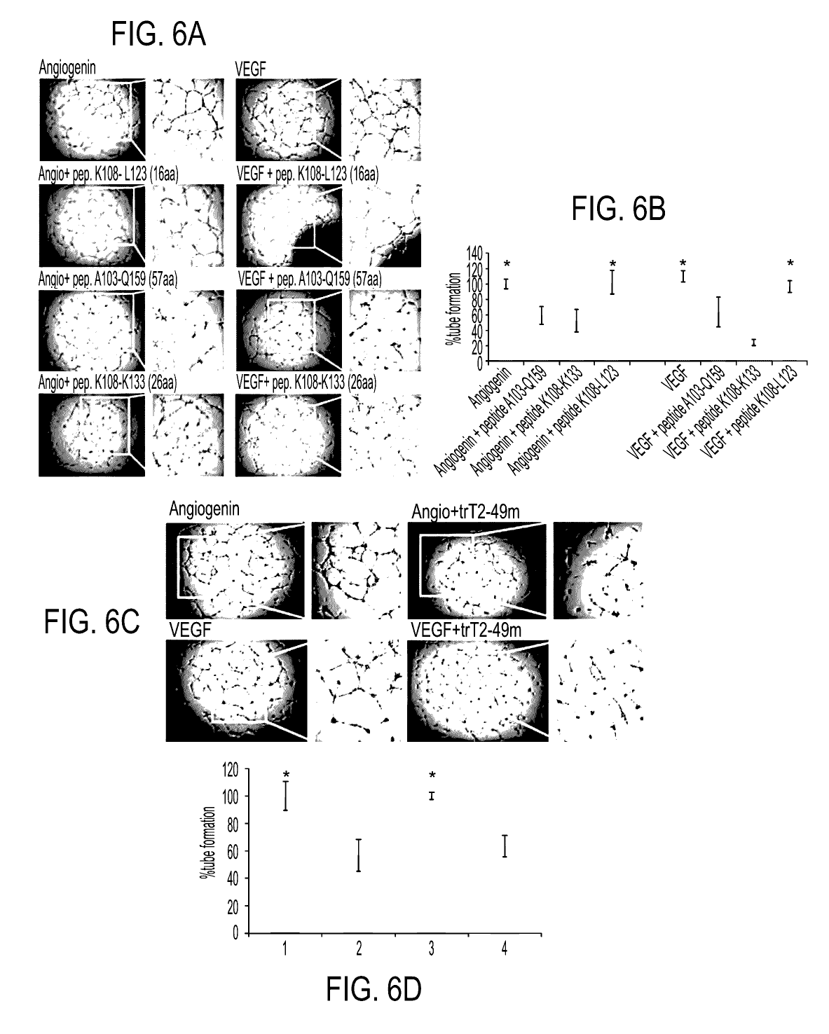

The antiangiogenic effect of trT2-49m (SEQ ID NO: 129) and peptides A103-Q159 (SEQ ID NO: 60) and K108-K133 (SEQ ID NO: 57) was assessed in a HUVEC tube formation assay. The antiangiogenic effect was compared to the effect of peptide K108-L123 (SEQ ID NO: 67, 16 aa) that failed to bind actin and to a control (PBS). Treatment with 2 μM peptide A103-Q159 (SEQ ID NO: 60) led to statistically significant ˜40% inhibition (P<0.05) of angiogenin- and VEGF-induced tube formation compared to the Control (FIGS. 6A and 6B). Treatment with peptide K108-K133 (SEQ ID NO: 57) led to statistically significant ˜50% inhibition (P<0.05) of angiogenin-induced tube formation (FIGS. 6A and 6B) and ˜75% inhibition of VEGF-induced tube formation compared to the Control (FIGS. 6A and 6B). Peptide K108-L123 (SEQ ID NO: 67), in addition to failing to bind actin, was also unable to inhibit tube formation (FIGS. 6A and 6B). Treatment with trT2-49m led to statistically significant 40% inhibition (P<0.05) of angiogenin-induced tube formation (FIGS. 6C and 6D) and to statistically significant 35% inhibition (P<0.05) of VEGF-induced tube formation (FIGS. 6C and 6D), (N=5 for each treatment).

Human RNASET2 Peptides Inhibit Blood Vessels Formation in an Ex Ovo CAM Assay, Similar to Full Length and Truncated Human RNASET2

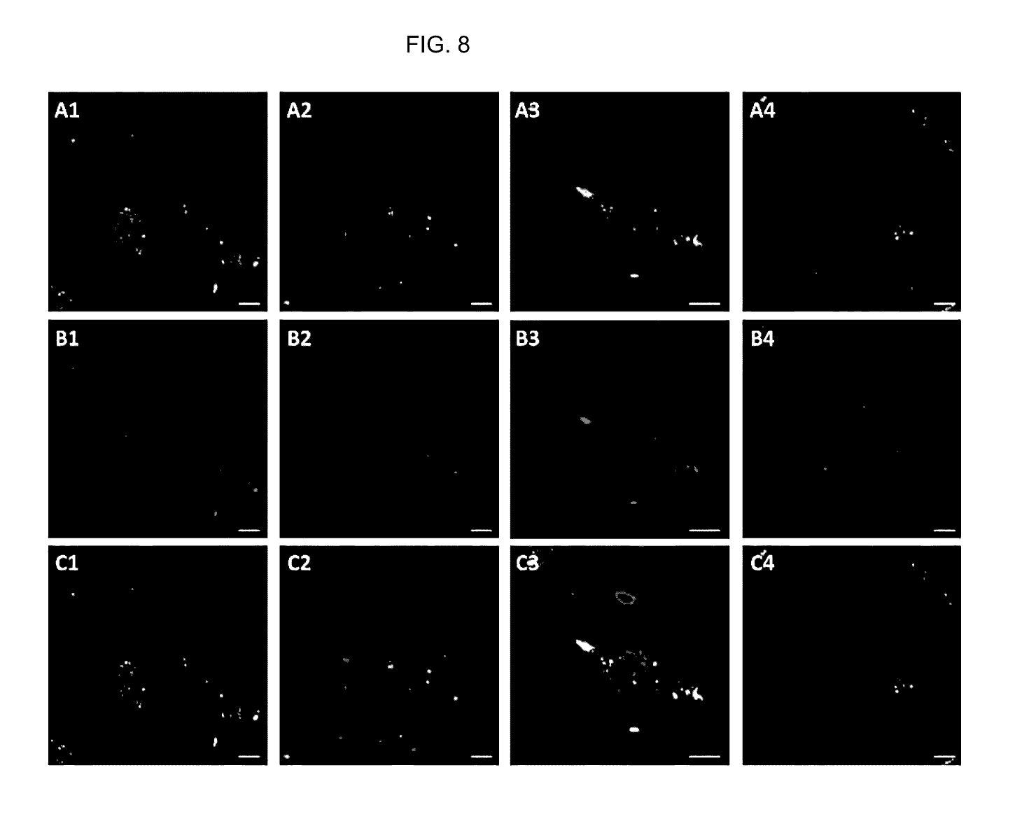

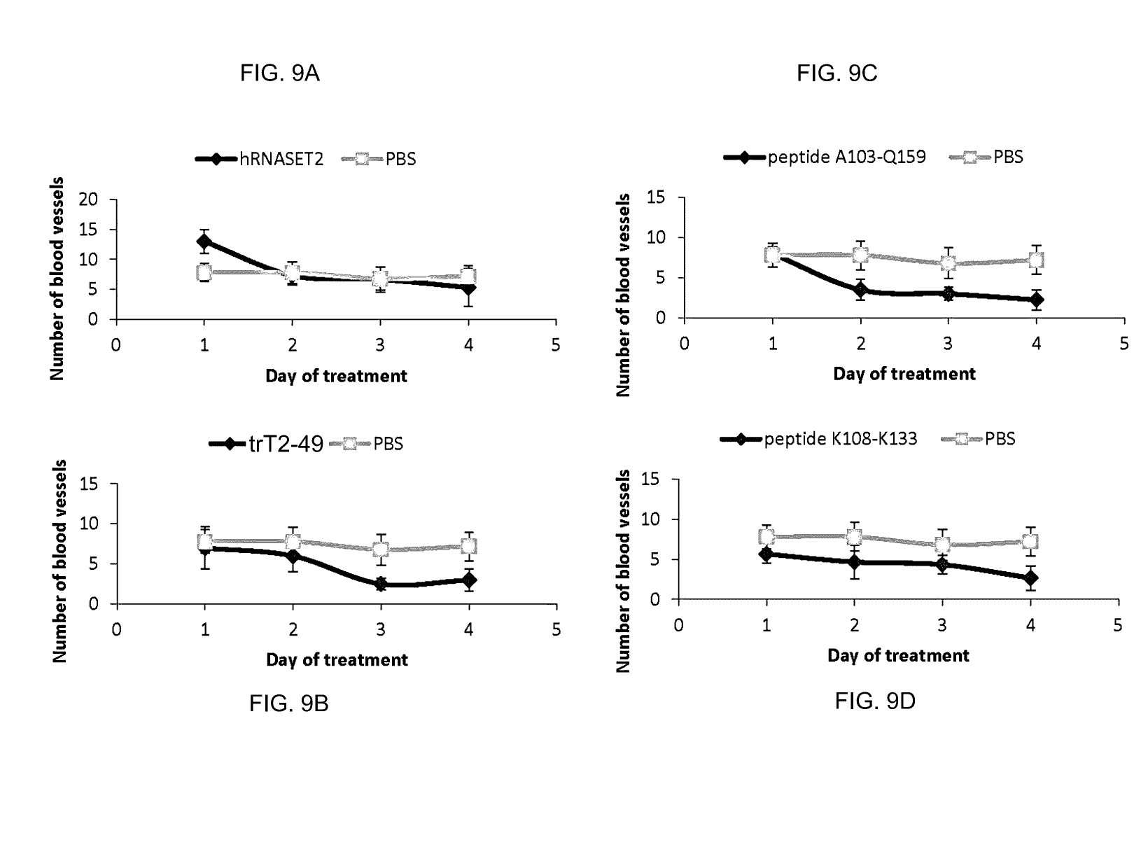

In this in-vivo assay (fertilized embryo is growing) the embryos develop a vascular network, and inhibition can be easily visualized. After eight days of incubation, treatment with human RNASET2 peptides revealed that all of the actin-binding RNASET2 peptides tested (SEQ ID NO: 1, SEQ ID NO: 84, SEQ ID NO: 60 and SEQ ID NO: 57) were effective in inhibiting angiogenesis (fewer blood vessels observed, compared to control) (FIGS. 9A-9D).

Immunofluorescence Human RNASET2 Peptide Rapidly Internalizes into the Cell

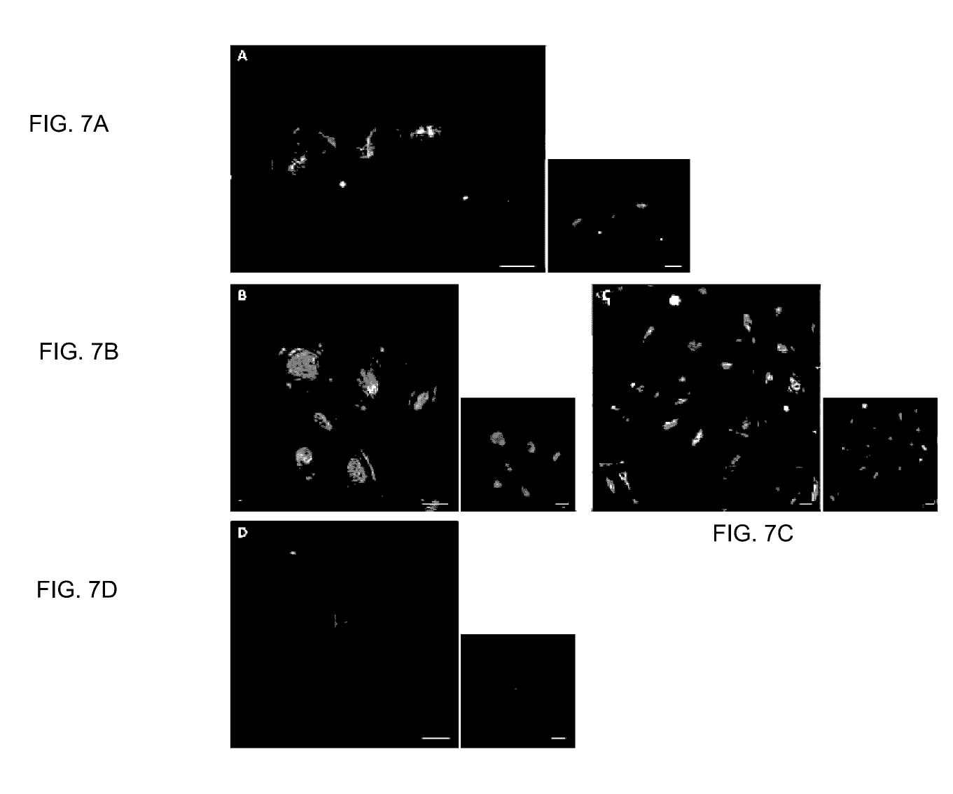

When labeled with green fluorescence, peptide K108-K133 (SEQ ID NO: 57) was observed in the cytoplasm surrounding the nucleus of HUVE cells in the Matrigel™ assay (FIGS. 7A-7D). The peptide accumulated in the cytoplasm after 2 (FIG. 7B) and 8 (FIG. 7C) hours, but after 24 h (FIG. 7D) the cellular signal was less pronounced.

Human RNASET2 Peptides Co-Localize with Angiogenin

In the presence of peptide K108-K133 (SEQ ID NO: 57) and angiogenin co-localization of both molecules was observed (FIGS. 8A1-8C4), similar to the co-localization previously observed with trT2-49 (SEQ ID NO: 84) and angiogenin. While not intending to be bound by any particular theory, this observation suggests that both the peptides and the angiogenin bind (or compete) for similar cellular epitopes as previously reported for ACTIBIND. Moreover, the addition of angiogenin to the cultured cells resulted in a reduction of the peptide signal inside the cell compared with the signal observed for the peptide alone (compare FIGS. 7A-7D with FIGS. 8A1-C4).

Taken together, these results indicate that actin binding capacity correlates well with the in-vitro and in-vivo anti-angiogenic effects of the human RNASET2 peptides of the invention. trT2-49m (SEQ ID NO: 129), lacking the previously reported putative actin binding peptide (SEQ ID NO: 67) of Kumar et al and Gundampati et al, and some of the shorter RNASET2 peptides are able to bind actin and concomitantly inhibit angiogenesis. In addition, immunofluorescent labeled RNASET2 peptide K108-K133 (SEQ ID NO: 57, 26aa) rapidly undergoes translocation towards the cell cytoplasm and nucleus of HUVECs.

Using solid-phase actin binding assay (ELISA) and BIAcore analysis, it was observed that trT2-49 (SEQ ID NO: 84), trT2-49m (SEQ ID NO: 129), peptide A103-Q159 (SEQ ID NO: 60) and peptide K108-K133 (SEQ ID NO: 57) bound actin with similar affinities. For the full length protein it was impossible to perform the ELISA assay probably due to steric disturbance, but in BIAcore analysis, the measured affinity was similar to the above molecules indicating that the three proteins (hRNASET2, trT2-49 and trT2-49m) and the two peptides (A103-Q159 SEQ ID NO: 60 and K108-K133 SEQ ID NO: 57) are similar in their actin-binding ability.

While not intending to be bound by any one particular theory, it is possible that hRNASET2 or its actin-binding peptide derivatives may compete with proteins which play a central role in angiogenesis and tumor progression (such as angiogenin) through binding of endothelial cell surface actin in endothelial cells, thereby inhibiting cell invasiveness and migration.

### Peptide Array Screen for Actin-Binding Human RNASET2 Peptides

Peptide arrays are powerful tools for characterizing protein interactions and identifying specific domains involved in mediating these interactions. A peptide array based on overlapping peptides of the sequence of human RNASET2 was designed and used to assay actin binding of immobilized human RNASET2 peptides.

### Materials and Methods

An array of 192 partially overlapping RNASET2 derived peptides, modified peptides based on the human RNASET2 peptide K108-K133 sequence (SEQ ID NO: 57) and actin derived peptides was synthesized by INTAVIS Bioanalytical Instruments AG, Koeln, Germany. The peptides were acetylated at their N-termini and attached to a cellulose membrane via their C-termini through an amide bond. Peptide array (INTAVIS Bioanalytical Instruments AG, Koeln, Germany).

### Materials:

Rabbit muscle actin (A2522, Sigma-Aldrich, St Louis, Mo.);

TBST Buffer: 50 mM Tris HCl pH=7.5, 150 mM NaCl, 0.05% Tween20;

Skim milk;

Blocking solution: TBST Buffer with 2.5% milk;

Rabbit anti-actin antibody (A2066 Sigma-Aldrich, St Louis, Mo.);

Peroxidase-conjugated affinipure goat anti rabbit IgG 111-035-003 (Jackson Laboratories, Bar Harbor Minn.).

The array was immersed in 5 ml blocking solution (TBST containing 2.5% milk) for 4 hours with shaking, then washed once with blocking solution (30 sec.) and twice with TBST (5 min. each). 5 μM actin were dissolved in the blocking solution (˜1:3), and incubated with the array at 4° C. with shaking overnight. After washing with TBST (1×30 sec. and 2×5 min.) the array was incubated first with rabbit anti actin antibody diluted 1:100 in blocking solution for 4 hours at room temperature followed by washings three times with TBST (1×30 sec. and 2×5 min.). The array was then incubated with horseradish peroxidase-conjugated goat anti rabbit IgG antibody diluted 1:10,000 in blocking solution at room temperature for 2 hours followed by washing with TBST (1×30 sec. and 2×5 min.). Immunodetection was performed using chemiluminiscence with ECL reagents.

Results

The array containing a library of human RNASET2 modified peptides derived from the sequences of peptide K108-K133 (SEQ ID NO: 57). The modifications included an alanine scan, truncations and systematic replacements of each residue in each peptide by the corresponding D-amino acid residue and N-methylated residue, C terminal truncations and N terminal truncations. The array was screened for binding the actin protein. Intensity of the spots for all peptides was estimated by densitometry and compared to that of the unmodified RNASET2 peptide K108-K133 (SEQ ID NO: 57).

Table 6 shows the immobilized peptides with strong actin binding, relative to RNASET2 peptide K108-K133 (SEQ ID NO: 57). Note the predominance of helix 5 sequences in the strongest actin-binding peptides.

TABLE 6

| | PEPTIDE COORDINATES & | |
|---|---|---|
| SEQUENCE | SEQ ID NO. | DESCRIPTION |
| KKYFGRSLELYRELDLNSVLLKLGIK | Pep.K108-K133 | Unmodified peptide |
| | (SEQ ID NO: 57) | K108-K133 (26aa) |
| KKYFGRSLELYRELDLNSVLLKL_{(D)}GIK | Pep. K108-K133 D-Leu130 | D-amino acid scan |
| | (SEQ ID NO: 130) | |
| KKYFGRSLELYR | Pep. K108-R119 | C term. Truncations |
| | (SEQ ID NO: 62) | |
| KKYFGRSLEL | Pep. K108-L117 | C term. Truncations |
| | (SEQ ID NO: 131) | |
| LYREL | Pep. L117-L121 | Middle R |
| | (SEQ ID NO: 132) | |
| KKYFGRSLELYRADFKDALARVYG | Pep. K108-K119 + | Helix 5 and 7 |
| | A142-G153 | |
| | (SEQ ID NO: 133) | |

Thus, in addition to the longer RNASET2 peptides identified by ELISA (see Example I, above), short actin-binding RNASET2 peptides, comprising sequences from helix 5 of RNASET2 (e.g. SEQ ID NOs. 62, 131 and 133), and pharmaceutical compositions comprising the same can have therapeutic potential, for example, in anti-angiogenic and anti-tumor applications. Further, as observed for peptide K108-K133 D-leu 130 (SEQ ID NO: 130), actin-binding peptides of the present invention having amino acid modifications can also have therapeutic potential, for example, in anti-angiogenic and anti-tumor applications.

### Example III

### Anti-Tumor Effect of Human RNASET2 Peptides in an In-Vivo Xenograft Model

Materials and Methods

Animals

Female Hds-athymic nude mice (Harlan, Rehovot, Israel) were housed in laminar flow cabinets under specific pathogen-free conditions. Experiments were performed on mice at 7-8 weeks of age. The Animals were maintained in facilities approved by the Ethics Committee for Animal Experimentation, The Israeli Ministry of Health.

Xenograft Tumor Model

Subcutaneous tumors were produced by injecting 2.5×10⁵ Super metastatic A375SM cells (single-cell suspensions, >95% viability by a trypan blue exclusion test) in 0.2 ml of Hanks' buffered salt solution into the left flank of each mouse. Tumor growth was recorded three times weekly with a caliper and calculated as a×b²/2 cm³ (a, long diameter; b, short diameter). Treatments commenced when tumors arrived to about 100 mm³ volumes, as follows (iv=intravenous; it=intra-tumor, direct injection, q3×6=three times per week for six weeks, q7×6=daily for six weeks):

Group I: PBS as Control (100 ul/mouse, iv, q3×6),

Group II: Human RNASET2 peptide SEQ ID NO: 57 (2.5 mg/kg in 100 ul PBS/mouse, iv, q3×6);

Group III: Human RNASET2 peptide SEQ ID NO: 57 (5 mg/kg in 100 ul PBS/mouse, iv, q3×6);

Group IV: Human RNASET2 peptide SEQ ID NO: 57 (7.5 mg/kg in 100 ul PBS/mouse, iv, q3×6);

Group V: Human RNASET2 peptide SEQ ID NO: 57 (2.5 mg/kg in 100 ul PBS/mouse, iv, q7×6);

Group VI: PBS as Control (100 ul/mouse, it, q3×6),

Group VII: Human RNASET2 peptide SEQ ID NO: 57 (2.5 mg/kg in 100 ul PBS/mouse, it, q3×6)

The tumor diameters were measured three times per week.

Each group comprised 10 mice. At the end of experiment, tumors were excised, weighed, and prepared for histopathological examination.

Histopathology

Sections from the excised tumors were examined for athological changes, using Hematoxylin-Eosin staining (nuclei stain blue) and FragEL™ TdT DNA Fragmentation Detection stain (Calbiochem, EMD Millipore, MA) for visualizing apoptotic nuclei.

The FragEL™ DNA Fragmentation Detection Kit allows the recognition of apoptotic nuclei on tissue sections, cryo-sections and cells fixed on slides.

The enzyme terminal deoxynucleotidyl transferase (TdT) catalyzes the addition of biotin-labeled and unlabeled deoxynucleotides to the free 3′-OH groups at the ends of DNA fragments generated by the apoptotic endonucleases. Biotinylated nucleotides are then detected using a streptavidin-horseradish peroxidase conjugate and the reaction is then visualized with diaminobenzidine (DAB), H₂O₂ and urea. Finally, counterstaining is performed with methyl green. Apoptotic nuclei stain brown.

Results

Human RNASET2 Peptide Significantly Inhibits Tumor Growth

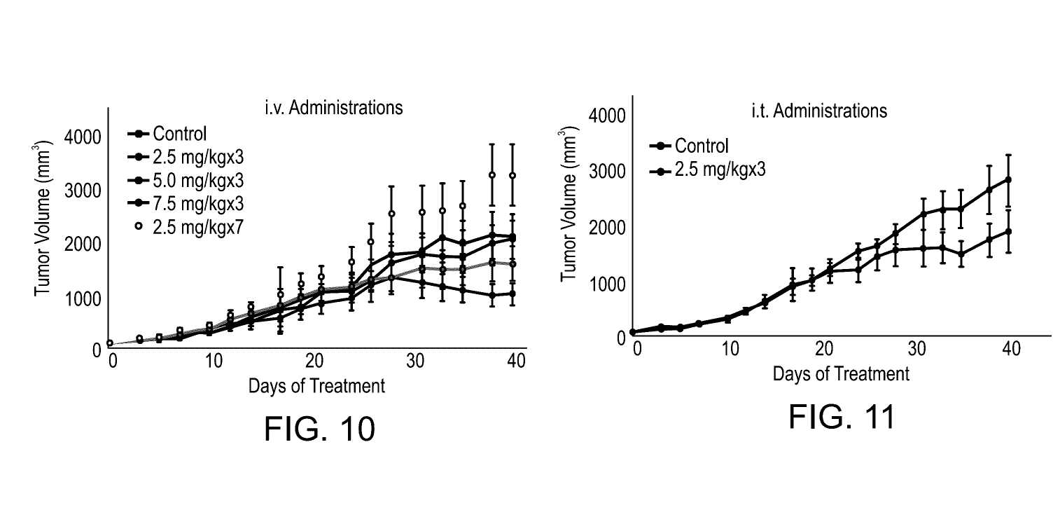

Tumor volume measurements showed that a daily intravenous (iv) treatment with human RNASET2 peptide K108-K133 (SEQ ID NO: 57), at a dose of 2.5 mg/kg, significantly (P<0.05) increased the rate of tumor development compared to control and all other treatments (FIG. 10). Within the groups receiving treatment with human RNASET2 peptide three times a week, a trend of increasing inhibition of tumor development can be discerned for dosage of 2.5 mg/kg (FIG. 10). However, statistical analysis (P<0.05) showed no significance between control and the different doses.

Increasing inhibition of tumor growth (measured by volume) compared to control was also observed with intra-tumor (it) treatment (FIG. 11), three times per week with human RNASET2 peptide K108-K133 (SEQ ID NO: 57), at a dose of 2.5 mg/kg, however statistical analysis showed that the difference was not significant.

When tumor weight was measured (Table 7), statistical analysis of the results confirmed the anti-tumor effects indicated by the trends of the tumor volume results: at a dose of 2.5 mg/kg, both intravenous and intratumor treatment with human RNASET2 peptide K108-K133 (SEQ ID NO: 57) significantly inhibited tumor growth.

TABLE 7

| Treatment | | Tumor Weight |
|---|---|---|
| Intravenous (iv) | Control iv | 1702 ± 394^{b} |
| | 2.5 mg/kg q3x6 iv | 987 ± 246^{b} |
| | 5 mg/kg q3x6 iv | 1798 ± 385^{b} |
| | 7.5 mg/kg q3x6 iv | 1471 ± 418^{b} |
| | 2.5 mg/kg q7x6 iv | 3469 ± 648^{a} |
| Intra-tumor (it) | Control it | 2746 ± 471^{a} |
| | 2.5 mg/kg q3x6 it | 1874 ± 354^{a} |

Tumor weight analysis.

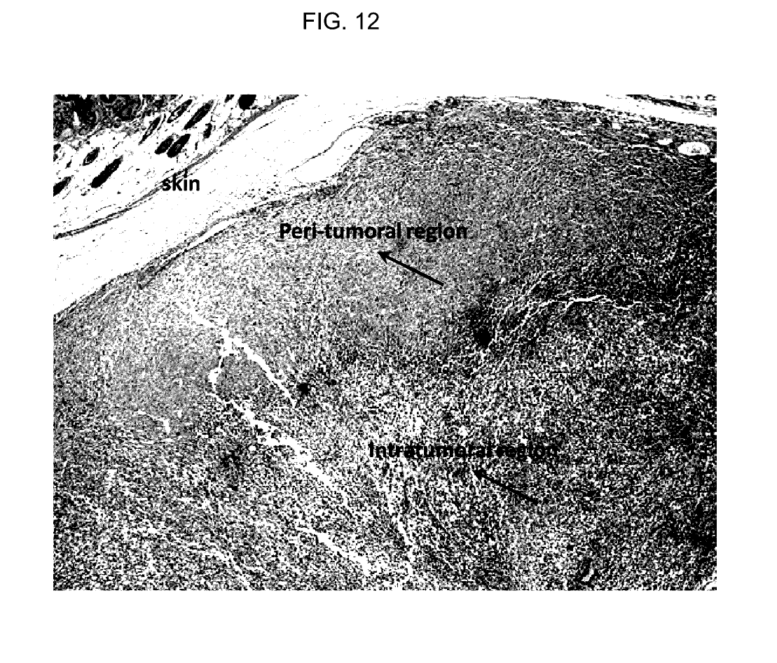

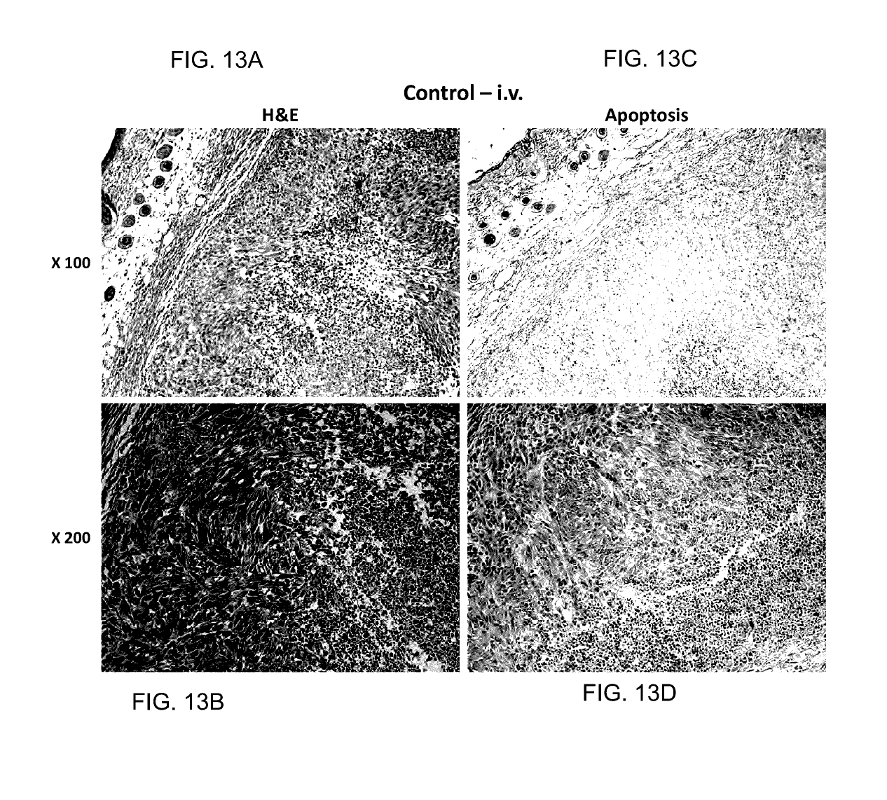

Histological analysis (H&E stain) of the tumor sections showed that apoptosis/necrosis generally occurs in the intratumoral region, as indicated by the morphology of the nuclei, localized in the inner part of the tumor section (FIG. 12, red arrow). The peri-tumoral region (FIG. 12, blue arrow), in contrast, is of normal appearing cells. Comparison of H&E staining (FIGS. 13A and 13B) with apoptosis-specific stain (FIGS. 13C and 13D) of sections from control (untreated) tumors reveals cells with generally euchromatic and proliferating nuclei, with only sporadic apoptotic nuclei (brown) detected, mostly in the intratumoral region.

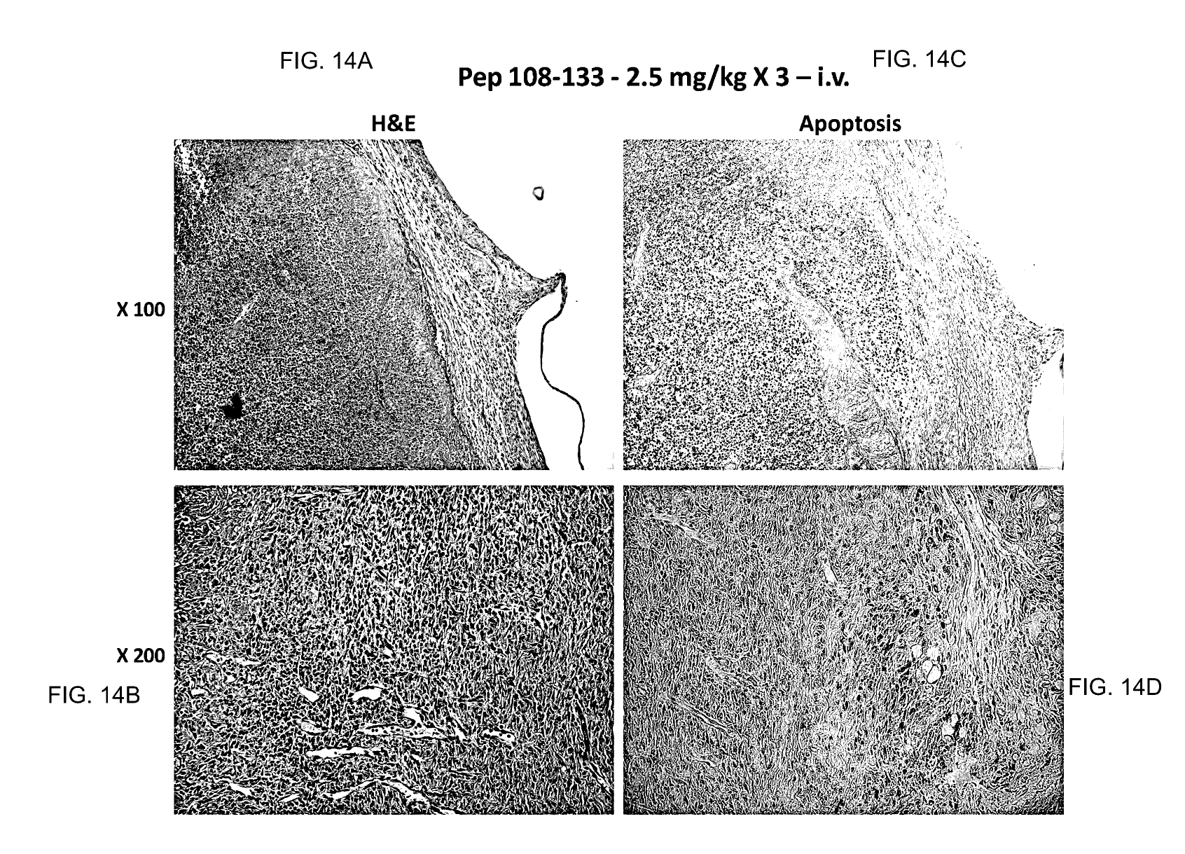

In stark contrast, H&E-stained sections from tumors from mice receiving intravenous treatment with 2.5 mg/kg human RNASET2 peptide K108-K133 (SEQ ID NO: 57), three times per week revealed compact, dense nuclei, mainly in the intratumoral region (FIGS. 14A and 14B). Apoptosis-specific stain (FIGS. 14C and 14D) of sections from the same tumors (FIGS. 14C and 14D) reveals abundant apoptotic cells (brown nuclei) cells.

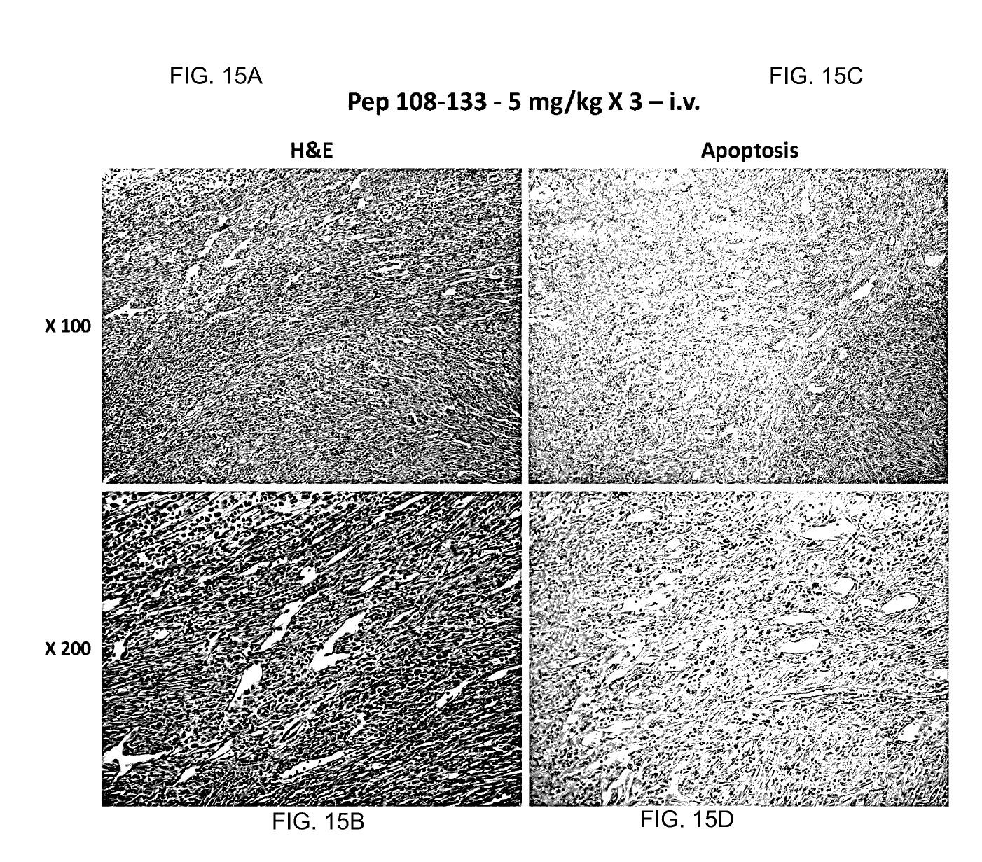

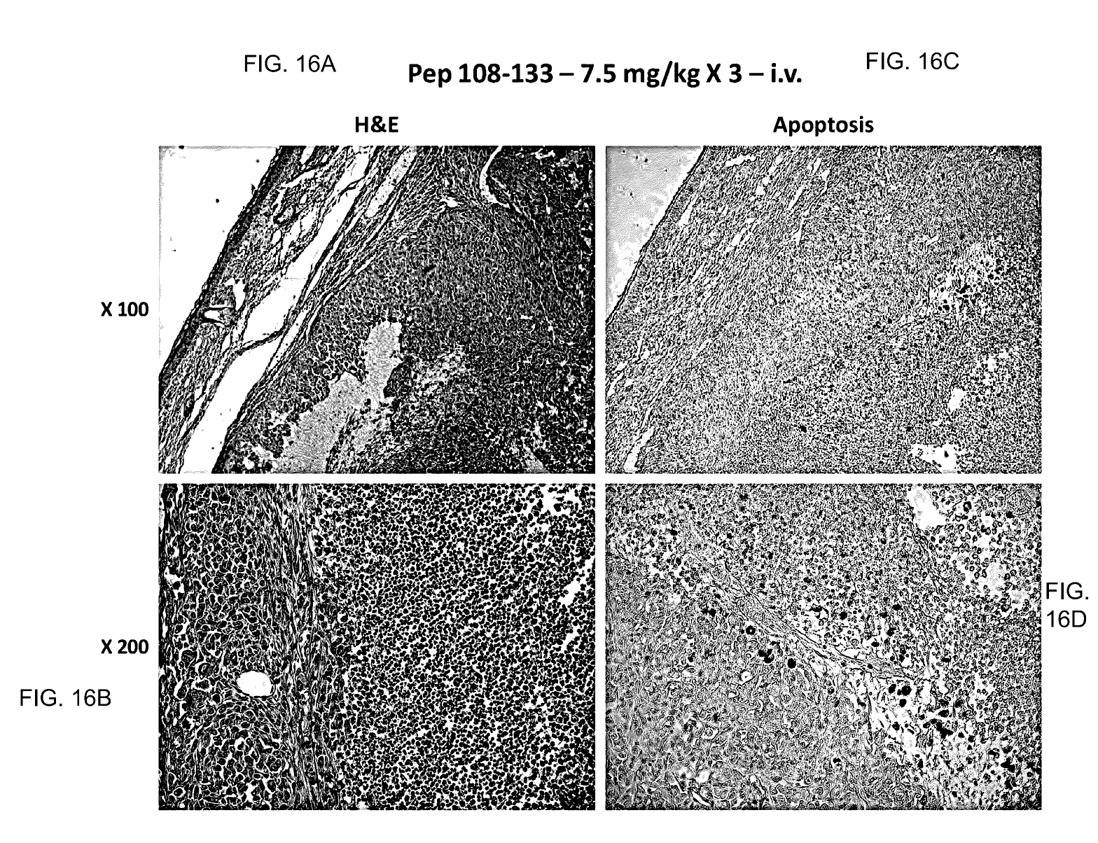

Increasing the dosage of the RNASET2 peptide to 5 mg/kg, three times per week administered intravenously resulted in much the same pattern of disrupted tumor histology (FIGS. 15A and 15B) and heightened apoptosis (FIGS. 15C and 15D) as observed with 2.5 mg/kg of the peptide. Further increasing the dosage to 7.5 mg/kg did not enhance or decrease the anti-tumor effects of the peptide (FIGS. 16A-16D).

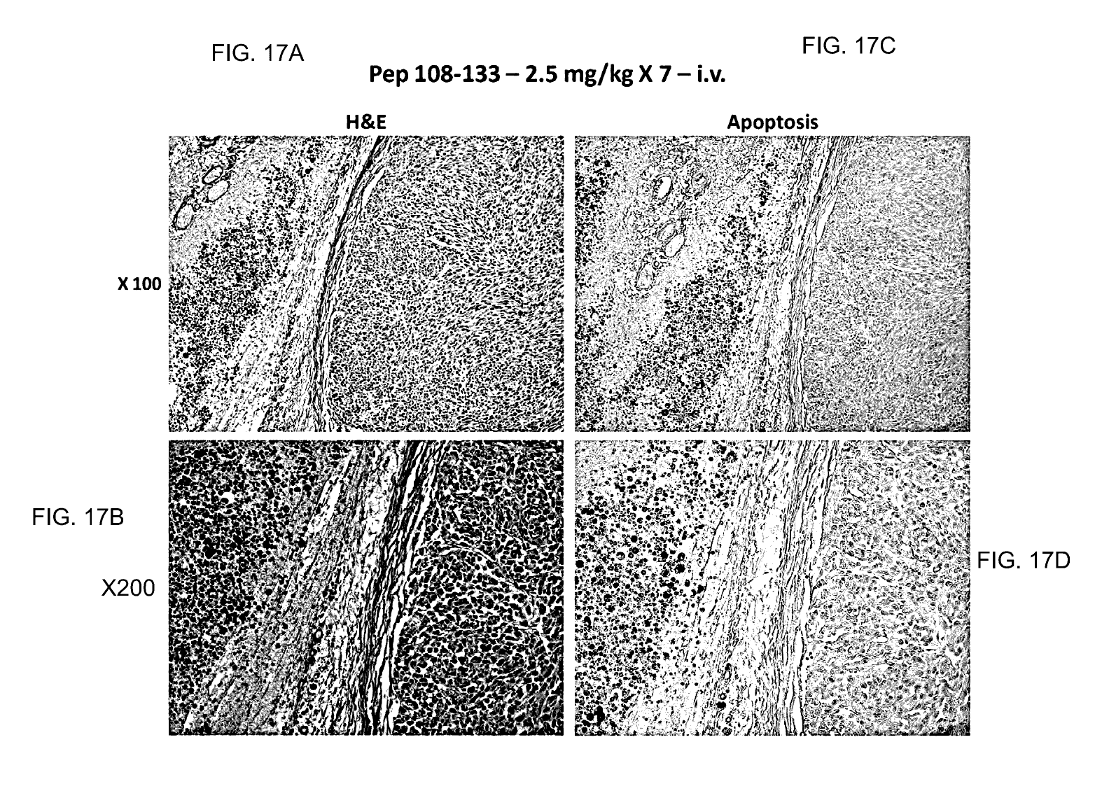

While the manual caliper measurements showed no apparent effect of the daily (Group V) intravenous administration of 2.5 mg/kg of Human RNASET2 peptide SEQ ID NO: 57, analysis of tumor sections revealed significant disruption of tumor histology (FIGS. 17A and 17B) and heightened apoptosis (FIGS. 17C and 17D), similar to that observed with the thrice weekly intravenous regimens.

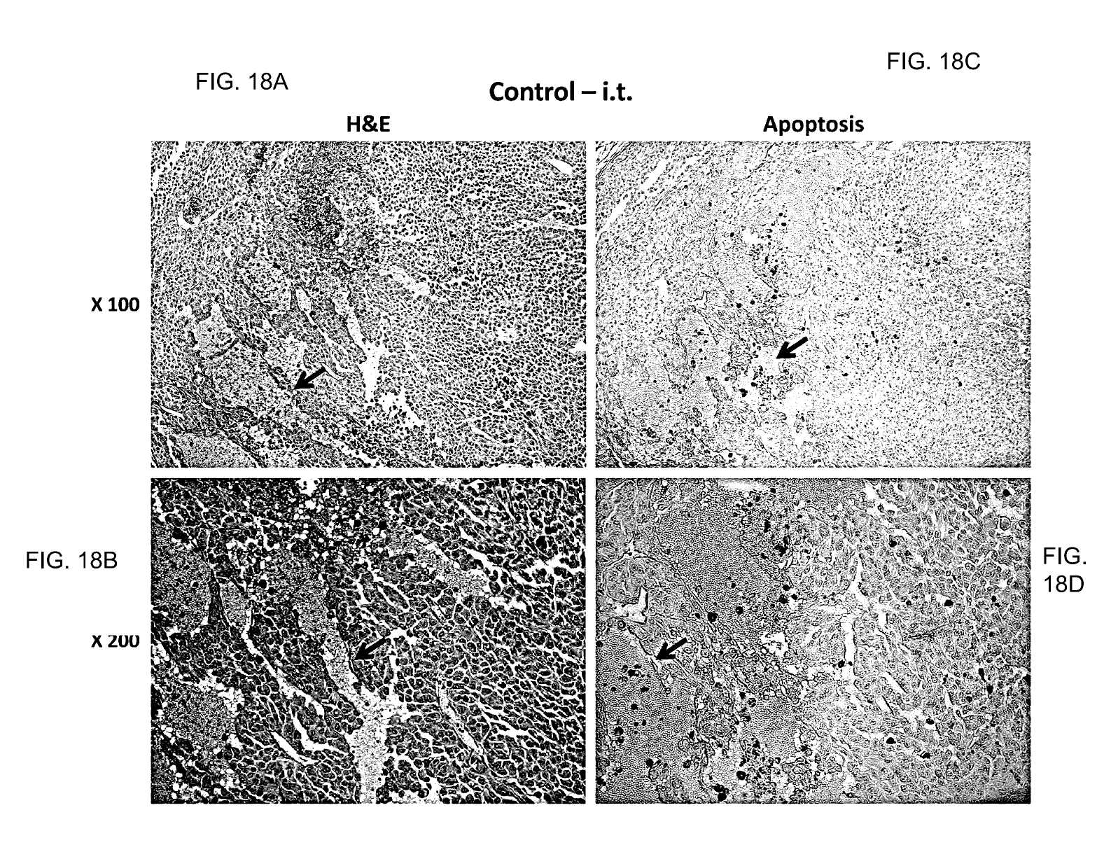

Intratumoral (direct) administration of PBS (control) to the tumors results in mechanical damage to the tumor tissue, evident in the tearing and hemorrhages (FIGS. 18A-18D, arrows) observed in both H&E and apoptotic stained sections. Note that, in these control sections, apoptotic nuclei (brown) appear only around areas of tissue damage (FIGS. 18C and 18D).

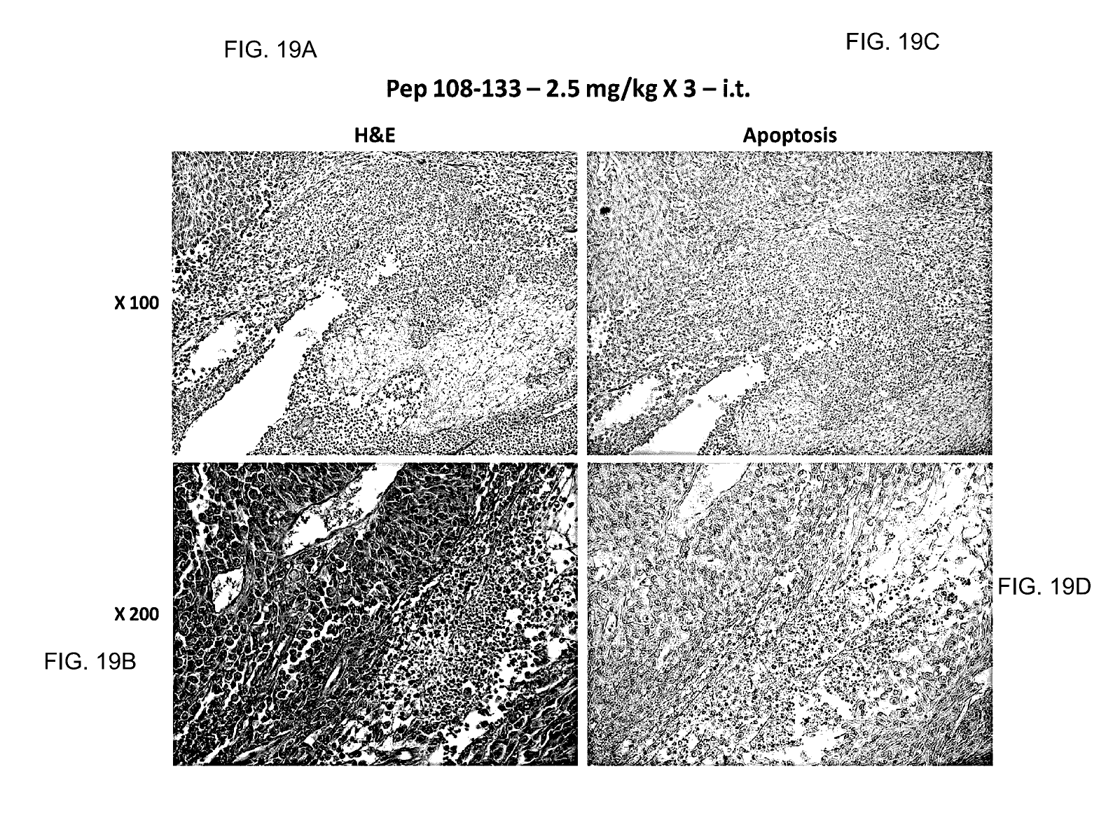

Similar mechanical disruption is evident with intratumoral (direct) administration of 2.5 mg/kg of the RNASET2 peptide, three times per week (FIGS. 19A-19D), however, abundant apoptosis (FIGS. 19C and 19D, brown nuclei) is also observed, similar to the patterns observed with intravenous administration of the peptide.

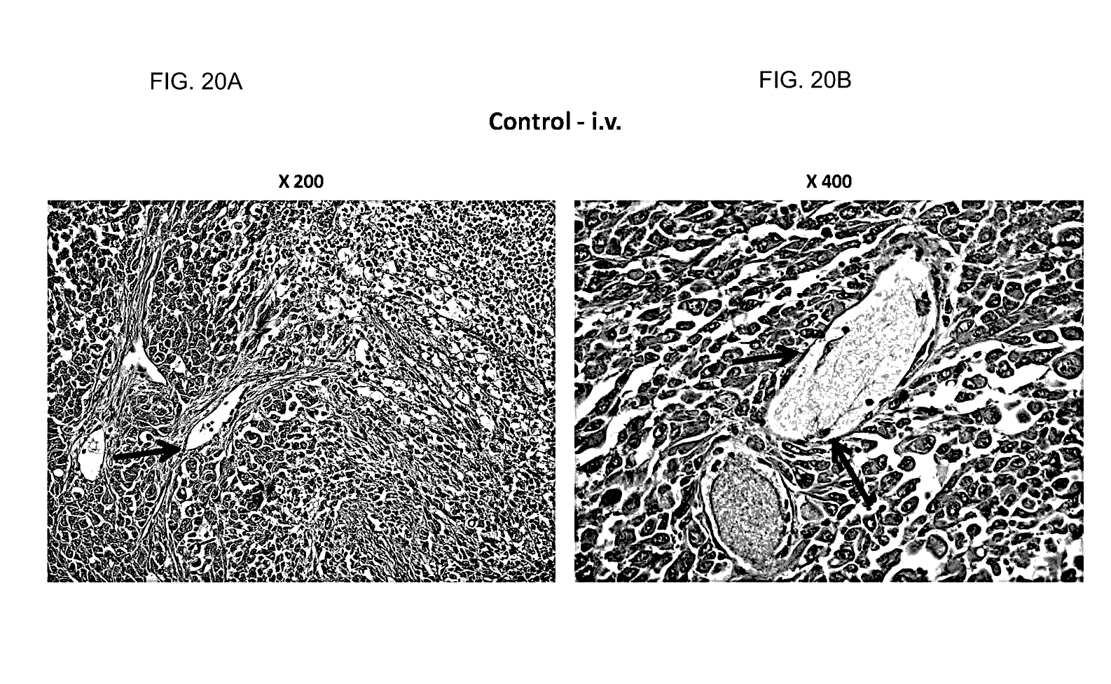

Observation of the vascular structures of tumors from mice treated with the human RNASET2 peptide reveals antiangiogenic effects of peptide administration on the tumor. In control tumor sections, tumor cells can be seen arranged in dense masses, with tumor cells adjacent to and touching blood vessels (blue arrows, FIGS. 20A and 20B) and intact vascular epithelial cells (red arrow, FIG. 20B).

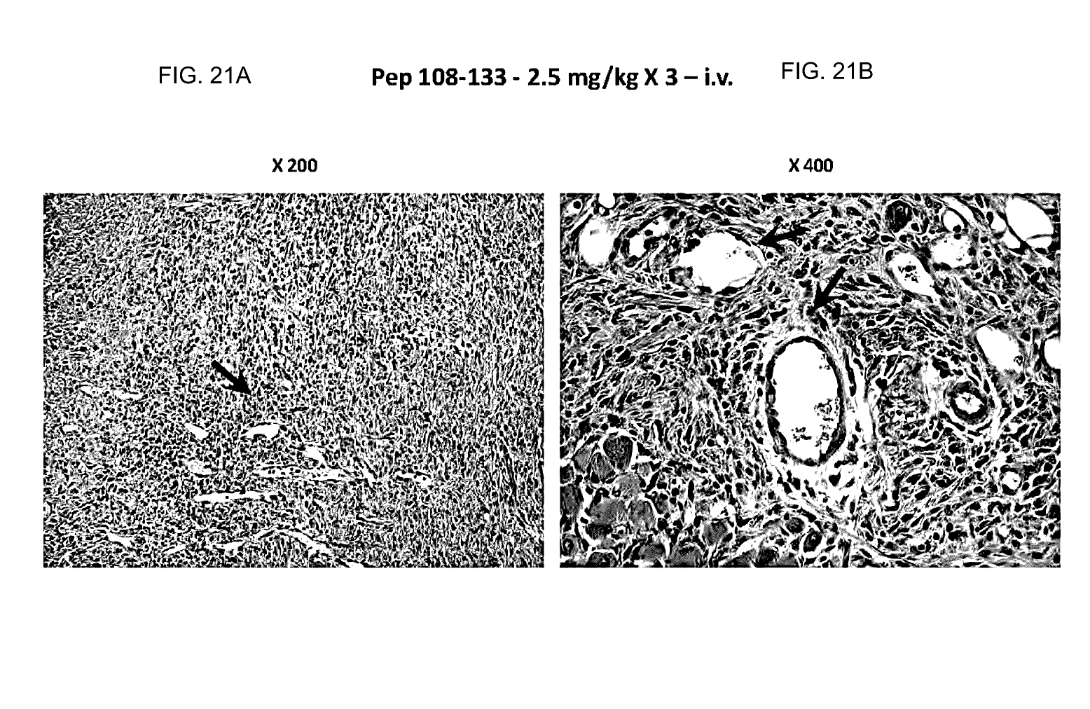

In stark contrast, H&E-stained sections from tumors from mice receiving intravenous treatment with 2.5 mg/kg human RNASET2 peptide K108-K133 (SEQ ID NO: 57), three times per week revealed numerous apoptotic cells (blue arrows, FIGS. 21A and 21B), accumulating towards the vascular structures, and visible disruption of the endothelial structure (red arrow, FIG. 21B).

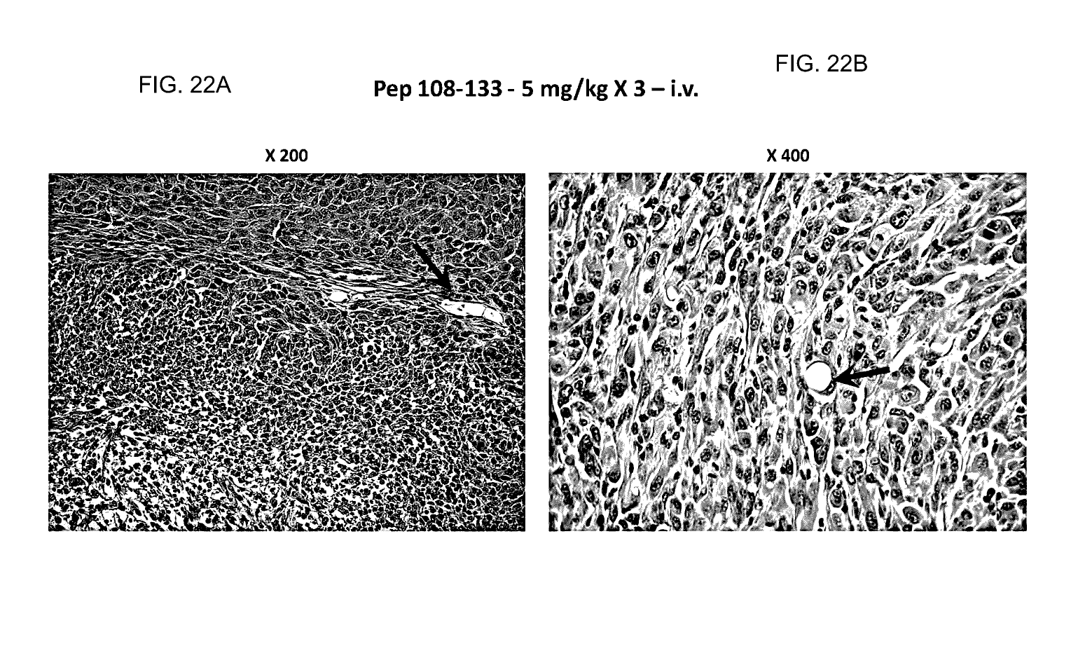

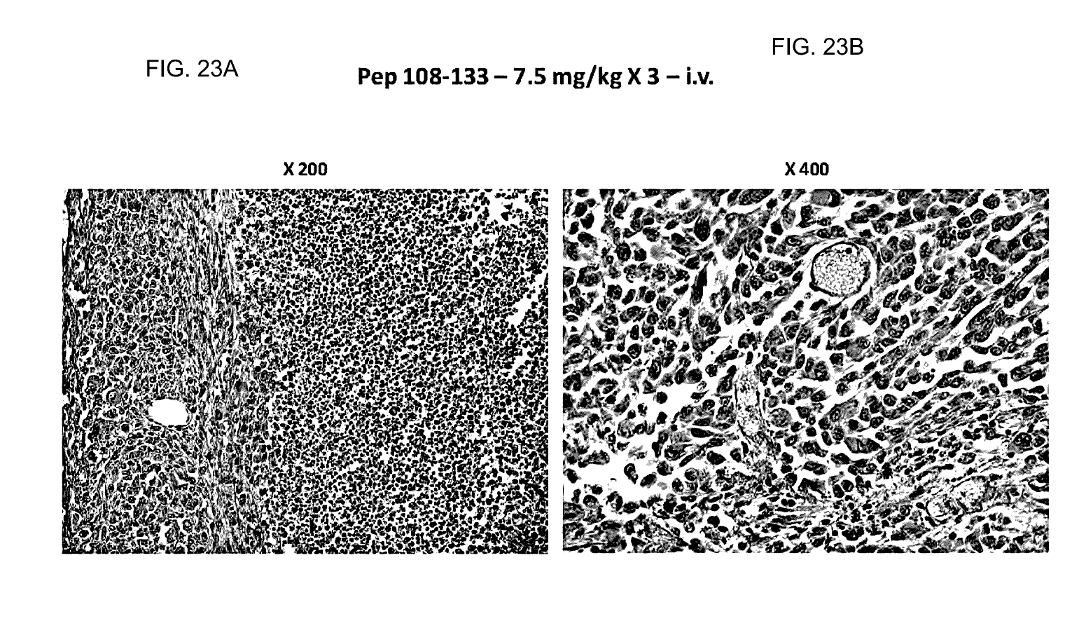

When the dosage is increased to 5 mg/kg human RNASET2 peptide K108-K133 (SEQ ID NO: 57), three times per week, many blood vessels can be seen surrounded by cancer cells (blue arrows, FIGS. 22A and 22B), with only partial disruption of the endothelial cell structures. A small, if any effect, relative to PBS controls, of further increasing the dosage to 7.5 mg/kg human RNASET2 peptide K108-K133 (SEQ ID NO: 57), three times per week is apparent (FIGS. 23A and 23B).

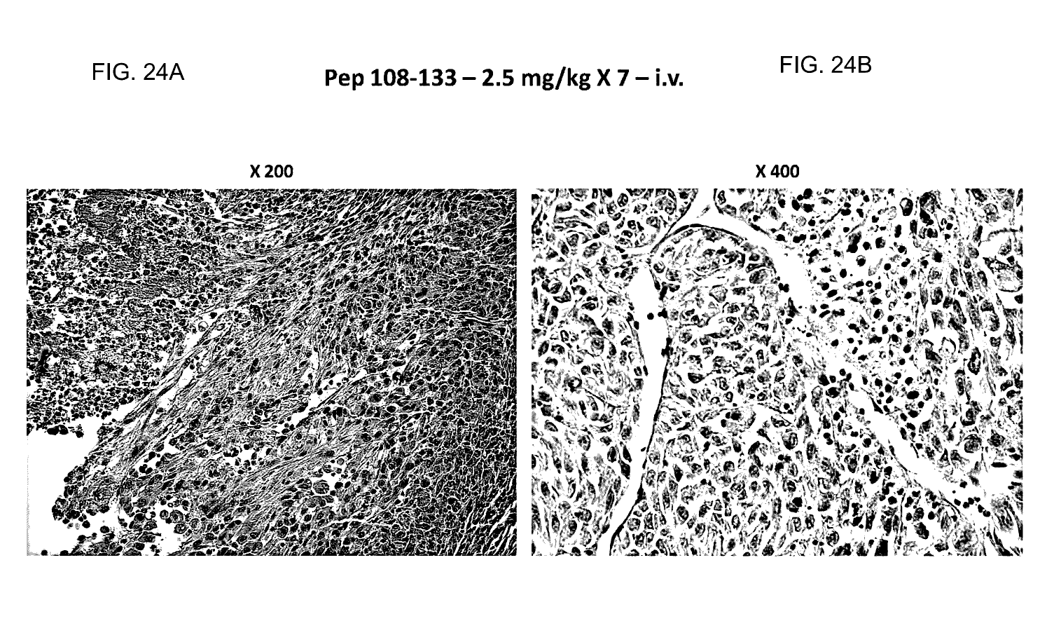

While the manual caliper measurements showed no apparent effect of the daily (Group V) intravenous administration of 2.5 mg/kg of Human RNASET2 peptide SEQ ID NO: 57, analysis of tumor sections through vascular structures revealed significant disruption of tumor histology and vascular structures (FIGS. 24A and 24B), similar to that observed with the thrice weekly intravenous regimens.

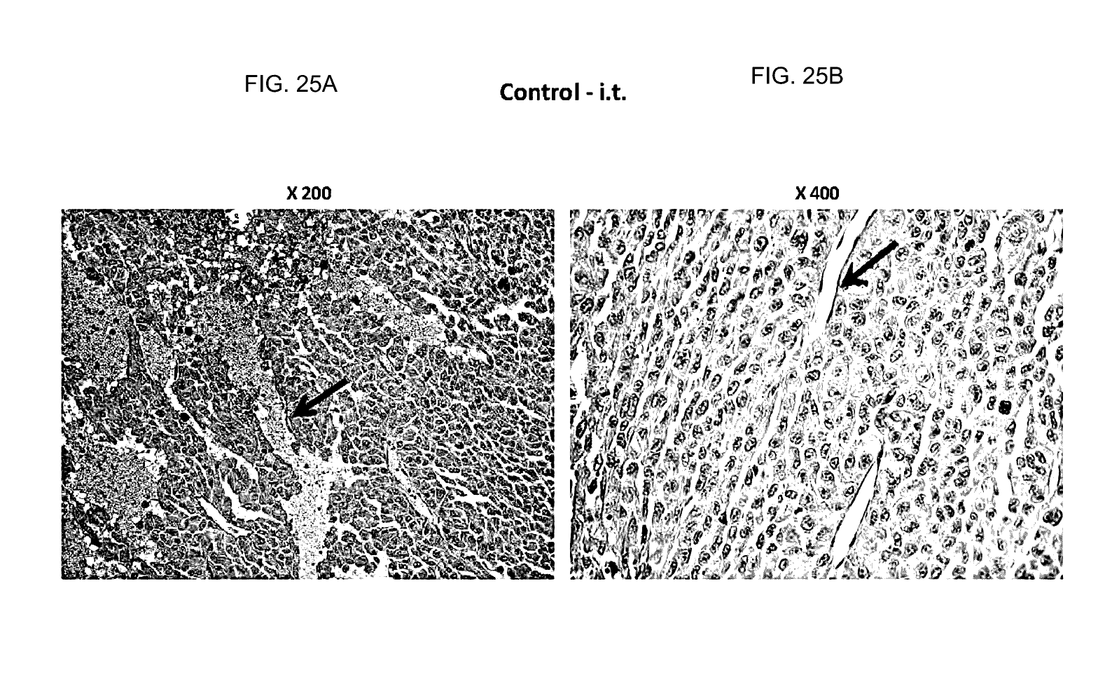

Intratumoral (direct) administration of PBS (control) to the tumors results in mechanical damage to the tumor tissue, evident in the tearing and hemorrhages (FIG. 25A, red arrow) observed. Note that, in these control sections, blood vessel endothelial cells are intact, and tumor cells are adjacent to and touching the vascular structures (blue arrow, FIG. 25B), similar to state of the tumor in control intravenous administration of PBS.

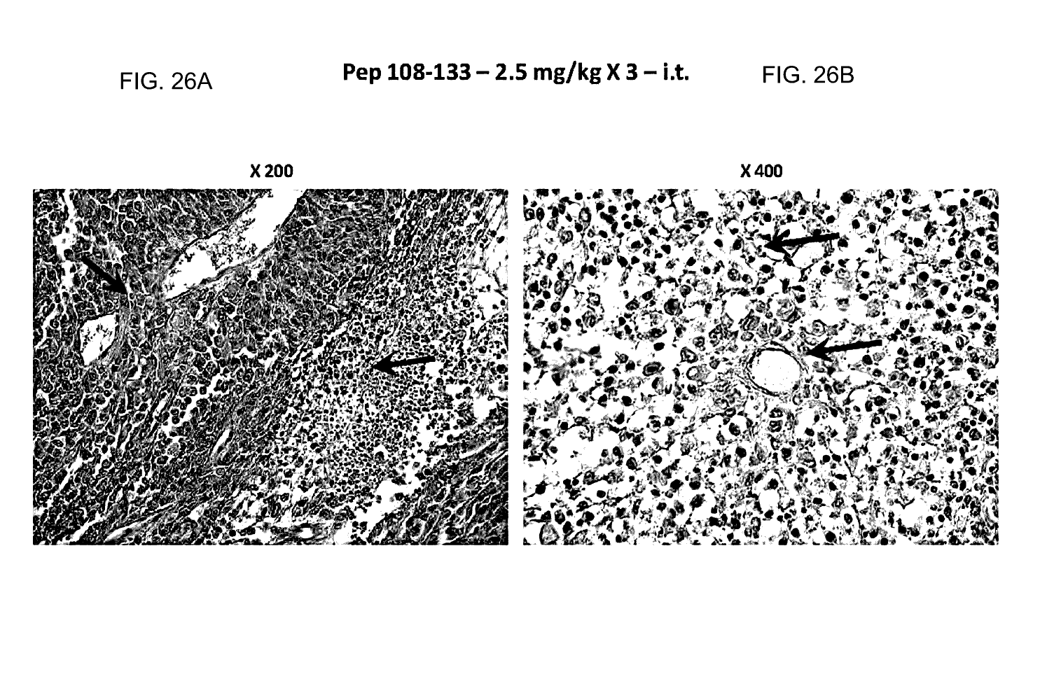

Similar mechanical disruption and hemorrhaging is evident with intratumoral (direct) administration of 2.5 mg/kg of the RNASET2 peptide, three times per week (FIGS. 26A-26B, black arrows), however, tumor cells can be seen accumulating towards the blood vessels (FIGS. 26A and 26B, red arrows).

Taken together, these results show that, upon histological analysis of the tumor sections, the gross anti-tumor effects observed with treatment of mice with human RNASET2 peptide (SEQ ID NO: 57) are reflected in significantly increased proportion of apoptotic tumor cells. Upon iv administration, but not with intratumoral administration, necrotic/apoptotic cells were observed accumulating towards the blood vessels, and tumor endothelial structures was visibly disrupted. Further, while a dose of 2.5 mg/kg, administered intravenously daily, led to an increase in tumor growth, relative to control and other treatments, the effect of daily administration on tumor apoptosis, as well as on tumor histology and vascular structures, was similar to the three-times-a-week intravenous regimen.

Still further, it was observed that increasing the dosage of the human RNASET2 peptide (SEQ ID NO: 57) to 5 and 7.5 mg/kg had no visible significant effect on tumor growth or on blood vessel histology, relative to control PBS administration. However, an increase in apoptotic rate, similar to all the above treatments, was observed with the increased dosages.

Thus, these results support a role for actin binding RNASET2 peptides of the present invention in anti-tumor and anti-angiogenic therapies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Trubia et al. 1997. Genomics 42:342-344
Acquati et al. 2001. Meth Mol Biol 160:87-101
Acquati et al. 2011, PNAS 108:1104-09
Smirnoff et al, 2006, Cancer 107:2760-69
Liu et al, 2002 Oncogene, 21:387-99
Luhtala and Parker, 2010 Trends Biochem Sci, 35:253-59
Schwartz et al, 2007, Canc Res 67:5258-66
Roiz et al, 2006 Cancer 106:2295-2308
de Leeuw et al, 2007 Acta Crystallogr Sect F Struct Biol Cryst. Communic, 63:716-719
MacIntosh, 2011, in Ribonucleases, Nucl Acids and Mol Biol 26, A. W. Nicholson, ed, pg 89-114.
Gundampati et al, 2012, J Mol Model 18:658-62
Kumar et al 2013, J Mol Model, 19:613-21
de Leeuw et al., 2012, J Med Chem 55:1013-20
Medinger et al 2010, J Angiogenesis Res 2:10
Rosca et al, 2011, Curr. Res Biotech, 12:1101
Campomenosi et al, 2006, Arch Biochem Biophys, 449:17-26
Mejean 1987 Biochem. J. 244: 571-577
Mejean 1992, Eur. J Biochem. 209: 555-562
Vannucci et al Int J Nanomed 2012; 7:1489-509
Roiz L et al. 2000. J Amer Soc Hort Sci. 125:9-14
Liotta, L. A., et al., 1991 Cell 64:327-336
Vasandani V. M., et al., 1996, Cancer Res. 15; 56 (18):4180-6
Nesiel-nuttman et al, Oncoscience 2014, Advanced Publication Nov. 26, 2014

1. An isolated peptide comprising a core amino acid sequence, which comprises at least 10 amino acids of helix 5 of human RNASET2, or naturally occurring homologues thereof, or conservative substitutions thereof, wherein the peptide is 23-50 amino acids in length and wherein the peptide binds actin.

2. The isolated peptide of claim 1, characterized by at least one of the following:
(a) said at least 10 amino acids of helix 5 of human T2RNase correspond to positions 108-121 of SEQ ID NO.: 1;
(b) amino acids of said core amino acid sequence corresponding to positions 116 and 122 of SEQ ID NO: 1 are negatively charged amino acids; and
(c) the amino acid of said core amino acid sequence corresponding to position 119 is a positively charged amino acid.

3-4. (canceled)

5. The isolated peptide of claim 1, comprising at least one additional amino acid sequence.

6. The isolated peptide of claim 5, wherein said at least one additional amino acid sequence is characterized by at least one of the following:
(a) comprises a human RNASET2 sequence or conservative amino acid substitutions thereof;
(b) comprises a homologous T2RNase sequence or conservative amino acid substitutions thereof;
(c) is of the same species as of said core sequence;
(d) is heterologous to the core sequence;
(e) comprises an amino acid sequence selected from the group consisting of SEQ ID NO: 25, SEQ ID NO: 26, SEQ ID NO:27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 and SEQ ID NO: 36, or a portion thereof;
(f) is positioned N-terminally to the core amino acid sequence;
(g) is positioned N-terminally to the core amino acid sequence; and
(h) comprises a helix.

7-13. (canceled)

14. The isolated peptide of claim 6, wherein said at least one additional amino acid sequence is a helix selected from the group consisting of SEQ ID NOs. 37, 38 and 39-56 of human T2RNASE.

15. The isolated peptide of claim 5, wherein said at least one additional amino acid sequence comprises at least two additional amino acid sequences, flanking said core amino acid sequence.

16. (canceled)

17. The isolated peptide of claim 2, characterized by at least one of the following:
(a) said core amino acid sequence is selected from the group consisting of SEQ ID NO: 2-24;
(b) said core amino acid sequence comprises SEQ ID NO: 127;
(c) said core amino acid sequence is as set forth in SEQ ID NO:81;
(d) said core amino acid sequence is as set forth in SEQ ID NO: 82;
(e) said core amino acid sequence is as set forth in SEQ ID NO: 83;
(f) said core amino acid sequence is as set forth in SEQ ID NO: 57;
(g) said core amino acid sequence is 23 amino acids in length, and
(h) said core amino acid sequence is 24 amino acids in length.

18-24. (canceled)

25. The isolated peptide of claim 1, comprising the amino acid sequence:
X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃ (SEQ ID NO: 128);
wherein X₁ and X₈ are selected from group E; X₂, X₄, X₁₅ and X₂₁ are selected from group A; X₃, X₉, X₁₉ and X₂₃ are selected from group C; X₅, X₇, X₁₁, X₁₃, X₁₆, X₁₇, X₁₈, X₂₀ and X₂₂ are selected from group D and X₆, X₁₀, X₁₂ and X₁₄ are selected from group B;
wherein group A consists of small, aliphatic, non-polar or slightly polar amino acid residues, group B consists of polar, negatively charged amino acid residues and their (uncharged) amides; group C consists of polar, positively charged amino acid residues, group D consists of large, aliphatic non-polar amino acid residues and group E consists of aromatic residues.

26-27. (canceled)

28. The isolated peptide of claim 25, comprising a sequence selected from the group consisting of SEQ ID NOs: 57, 81, 82, 83, 130 and 133.

29. The isolated peptide of claim 25, comprising SEQ ID NO: 57.

30. An isolated peptide comprising a core amino acid sequence, which comprises at least 5 amino acids of helix 5 of human RNASET2, or naturally occurring homologues thereof, or conservative substitutions thereof, wherein the peptide is 5-22 amino acids in length and wherein the peptide binds actin.

31. The isolated peptide of claim 30 selected from the group consisting of SEQ ID NO: 62, 131 and 132.

32. (canceled)

33. The isolated peptide of claim 1, having a biological activity other than actin binding.

34. The isolated peptide of claim 33, wherein said biological activity is characterized by at least one of:
(a) inhibition of angiogenesis; and
(b) prevention, inhibition and/or reversal of colonization, differentiation and/or development of abnormally proliferating cells, cell motility and metastatic transformation.

35. (canceled)

36. The isolated peptide of claim 34, wherein said abnormally proliferating cells are cancer cells.

37. A composition of matter comprising the isolated peptide of claim 1 formulated with a cell penetrating agent and/or a targeting moiety.

38. (canceled)

39. A nucleic acid construct comprising a polynucleotide encoding the peptide of claim 2 and a promoter element functional in mammalian cells.

40. (canceled)

41. A pharmaceutical composition comprising the isolated peptide of claim 2 and a pharmaceutically acceptable carrier.

42. (canceled)

43. A method of preventing, inhibiting and/or reversing colonization, differentiation and/or development of abnormally proliferating cells in a subject, and/or treating or preventing a proliferative disorder or disease in a subject, comprising administering a therapeutically effective amount of the isolated peptide of claim 2 to said subject.

44. (canceled)

45. The method of claim 43, wherein said proliferative disorder or disease is a metastatic disease.

46. (canceled)

47. A method of inhibiting angiogenesis in a subject, the method comprising administering to said subject a therapeutically effective amount of the isolated peptide of claim 2 to said subject.

48. The method of claim 47, wherein said angiogenesis is tumor angiogenesis.

49-50. (canceled)


SEQUENCE LISTING (133 sequences)

SEQ ID NO: 1 - protein, 232 aa, Homo sapiens
DKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 2 - protein, 15 aa, Homo sapiens
QKKYFGRSLELYREL

SEQ ID NO: 3 - protein, 14 aa, Otolemur garnetti
KKYFGKSLALYQKL

SEQ ID NO: 4 - protein, 14 aa, Canis lupus
KKYFGGSLDLYRDL

SEQ ID NO: 5 - protein, 14 aa, Equus caballus
KKYFGKSLDLYKEL

SEQ ID NO: 6 - protein, 13 aa, Sus scrofa
KYFGKTLDLYKEL

SEQ ID NO: 7 - protein, 14 aa, Felis catus
KRYFGGGLDLYQKL

SEQ ID NO: 8 - protein, 14 aa, Cricetulus griseus
KKYFGKSLDLYKQL

SEQ ID NO: 9 - protein, 14 aa, Mus musculus
KKYFGKSLDLYKQI

SEQ ID NO: 10 - protein, 13 aa, Rattus norvegicus
KYFGKSLDLYKQI

SEQ ID NO: 11 - protein, 14 aa, Gallus gallus
KKYFSKTLELYQLV

SEQ ID NO: 12 - protein, 14 aa, Crotalus adamanteus
KKYFQKALELYRKI

SEQ ID NO: 13 - protein, 14 aa, Anolis carolinensis
KKYFNKALELYKKL

SEQ ID NO: 14 - protein, 13 aa, Xenopus laevis
KYFSKGLEIYKQV

SEQ ID NO: 15 - protein, 13 aa, Danio rerio
KYFGKALELYHKF

SEQ ID NO: 16 - protein, 13 aa, Oncorhynchus keta
KYFGKVLELYHMV

SEQ ID NO: 17 - protein, 16 aa, Aspergillus niger
EEVQDFFQQVVDLFKT

SEQ ID NO: 18 - protein, 16 aa, Rhizopus niveus
EDIVDYFQKAMDLRSQ

SEQ ID NO: 19 - protein, 16 aa, Escherichia coli
DAYFGTMVRLNGEIKE

SEQ ID NO: 20 - protein, 14 aa, Lycopsersicum esculentum
QHAYFKKALDLKNG

SEQ ID NO: 21 - protein, 14 aa, Momordica charantie
QAAYFKLAVDMRNN

SEQ ID NO: 22 - protein, 15 aa, Nicotiana glutinosa
QHQYFKKALDLKNQI

SEQ ID NO: 23 - protein, 14 aa, Calystegia sepium
QYEYFSTTLMLYFK

SEQ ID NO: 24 - protein, 14 aa, Pyrus pyrifolia
ENHYFETVIKMYIS

SEQ ID NO: 25 - protein, 19 aa, Homo sapiens
DKRLRDNHEWKKLIMVQHW

SEQ ID NO: 26 - protein, 5 aa, Homo sapiens
PETVC

SEQ ID NO: 27 - protein, 26 aa, Homo sapiens
EKIQNDCRDPPDYWTIHGLWPDKSEG

SEQ ID NO: 28 - protein, 15 aa, Homo sapiens
CNRSWPFNLEEIKDL

SEQ ID NO: 29 - protein, 7 aa, Homo sapiens
LPEMRAY

SEQ ID NO: 30 - protein, 11 aa, Homo sapiens
WPDVIHSFPNR

SEQ ID NO: 31 - protein, 10 aa, Homo sapiens
SRFWKHEWEK

SEQ ID NO: 32 - protein, 14 aa, Homo sapiens
HGTCAAQVDALNSQ

SEQ ID NO: 33 - protein, 7 aa, Homo sapiens
LNSVLLK

SEQ ID NO: 34 - protein, 27 aa, Homo sapiens
PSINYYQVADFKDALARVYGVIPKIQC

SEQ ID NO: 35 - protein, 30 aa, Homo sapiens
LPPSQDEEVQTIGQIELCLTKQDQQLQNCT

SEQ ID NO: 36 - protein, 51 aa, Homo sapiens
KQDQQLQNCTEPGEQPSPKQEVWLANGAAESRGLRVCEDGPVFYPPPKKTK

SEQ ID NO: 37 - protein, 7 aa, Homo sapiens
LNSVLLK

SEQ ID NO: 38 - protein, 12 aa, Homo sapiens
VADFKDALARVY

SEQ ID NO: 39 - protein, 7 aa, Canis lupus
LNSMLQK

SEQ ID NO: 40 - protein, 7 aa, Sus scrofa
LNSTLQK

SEQ ID NO: 41 - protein, 7 aa, Felis catus
LNSMLQK

SEQ ID NO: 42 - protein, 7 aa, Cricetulus griseus
LNSVLLK

SEQ ID NO: 43 - protein, 7 aa, Mus musculus
LNSVLQK

SEQ ID NO: 44 - protein, 7 aa, Gallus gallus
LNGFLLK

SEQ ID NO: 45 - protein, 7 aa, Crotalus adamanteus
LNSFLLK

SEQ ID NO: 46 - protein, 7 aa, Anolis carolinensis
LNSYLLK

SEQ ID NO: 47 - protein, 6 aa, Xenopus laevis
LNSVLE

SEQ ID NO: 48 - protein, 6 aa, Danio rerio
LNSVLL

SEQ ID NO: 49 - protein, 6 aa, Oncorhynchus keta
LDGVMK

SEQ ID NO: 50 - protein, 11 aa, Aspergillus niger
SYTALSDAGIT

SEQ ID NO: 51 - protein, 8 aa, Rhizopus niveus
VYKAFSSN

SEQ ID NO: 52 - protein, 8 aa, Escherichia coli
EAGKFLAD

SEQ ID NO: 53 - protein, 8 aa, Lycopsersicum esculentum
LLSILGGA

SEQ ID NO: 54 - protein, 11 aa, Momordica charantie
IIGALRPHAAG

SEQ ID NO: 55 - protein, 8 aa, Calystegia sepium
ISEILSES

SEQ ID NO: 56 - protein, 8 aa, Pyrus pyrifolia
VSRILSKA

SEQ ID NO: 57 - protein, 26 aa, Homo sapiens
KKYFGRSLELYRELDLNSVLLKLGIK

SEQ ID NO: 58 - protein, 22 aa, Homo sapiens
ELDLNSVLLKLGIKPSINYYQV

SEQ ID NO: 59 - protein, 7 aa, Homo sapiens
SINYYQV

SEQ ID NO: 60 - protein, 57 aa, Homo sapiens
ALNSQKKYFGRSLELYRELDLNSVLLKLGIKPSINYYQVADFKDALARVYGVIPKIQ

SEQ ID NO: 61 - protein, 17 aa, Homo sapiens
ALNSQKKYFGRSLELYR

SEQ ID NO: 62 - protein, 12 aa, Homo sapiens
KKYFGRSLELYR

SEQ ID NO: 63 - protein, 39 aa, Homo sapiens
ALNSQKKYFGRSLELYRELDLNSVLLKLGIKPSINYYQV

SEQ ID NO: 64 - protein, 13 aa, Homo sapiens
KKYFGRSLELYRE

SEQ ID NO: 65 - protein, 14 aa, Homo sapiens
KKYFGRSLELYREL

SEQ ID NO: 66 - protein, 15 aa, Homo sapiens
KKYFGRSLELYRELD

SEQ ID NO: 67 - protein, 16 aa, Homo sapiens
KKYFGRSLELYRELDL

SEQ ID NO: 68 - protein, 17 aa, Homo sapiens
KKYFGRSLELYRELDLN

SEQ ID NO: 69 - protein, 18 aa, Homo sapiens
KKYFGRSLELYRELDLNS

SEQ ID NO: 70 - protein, 19 aa, Homo sapiens
KKYFGRSLELYRELDLNSV

SEQ ID NO: 71 - protein, 20 aa, Homo sapiens
KKYFGRSLELYRELDLNSVL

SEQ ID NO: 72 - protein, 21 aa, Homo sapiens
KKYFGRSLELYRELDLNSVLL

SEQ ID NO: 73 - protein, 22 aa, Homo sapiens
KKYFGRSLELYRELDLNSVLLK

SEQ ID NO: 74 - protein, 23 aa, Homo sapiens
KKYFGRSLELYRELDLNSVLLKL

SEQ ID NO: 75 - protein, 23 aa, Homo sapiens
KKYFGRSLELYRENSVLLKLGIK

SEQ ID NO: 76 - protein, 22 aa, Homo sapiens
GRSLELYRELDLNSVLLKLGIK

SEQ ID NO: 77 - protein, 21 aa, Homo sapiens
RSLELYRELDLNSVLLKLGIK

SEQ ID NO: 78 - protein, 20 aa, Homo sapiens
SLELYRELDLNSVLLKLGIK

SEQ ID NO: 79 - protein, 25 aa, Homo sapiens
KKYFGRSLELYRELDLNSVLLKLGI

SEQ ID NO: 80 - protein, 24 aa, Homo sapiens
KKYFGRSLELYRELDLNSVLLKLG

SEQ ID NO: 81 - protein, 25 aa, Homo sapiens
KYFGRSLELYRELDLNSVLLKLGIK

SEQ ID NO: 82 - protein, 24 aa, Homo sapiens
YFGRSLELYRELDLNSVLLKLGIK

SEQ ID NO: 83 - protein, 23 aa, Homo sapiens
FGRSLELYRELDLNSVLLKLGIK

SEQ ID NO: 84 - protein, 184 aa, Homo sapiens
EGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 85 - DNA, 78 bp, Homo sapiens
aagaagtactttggcagaagcctggaactctacagggagctggacctcaacagtgtgcttctaaaattggggataaaa

SEQ ID NO: 86 - DNA, 75 bp, Homo sapiens
aagtactttggcagaagcctggaactctacagggagctggacctcaacagtgtgcttctaaaattggggataaaa

SEQ ID NO: 87 - DNA, 72 bp, Homo sapiens
tactttggcagaagcctggaactctacagggagctggacctcaacagtgtgcttctaaaattggggataaaa

SEQ ID NO: 88 - DNA, 69 bp, Homo sapiens
tttggcagaagcctggaactctacagggagctggacctcaacagtgtgcttctaaaattggggataaaa

SEQ ID NO: 89 - protein, 11 aa, Artificial sequence
  Artificial
YGRKKRRQRRR

SEQ ID NO: 90 - protein, 9 aa, Artificial sequence
  Artificial
RKKRRQRRR

SEQ ID NO: 91 - protein, 34 aa, Artificial sequence
  Artificial
DAATATRGRSAASRPTERPRAPARSASRPRRPVE

SEQ ID NO: 92 - protein, 16 aa, Artificial sequence
  Artificial
RRWRRWWRRWWRRWRR

SEQ ID NO: 93 - protein, 7 aa, Artificial sequence
  Artificial
RRRRRRR

SEQ ID NO: 94 - protein, 10 aa, Artificial sequence
  Artificial
RPKKRKVRRR

SEQ ID NO: 95 - protein, 11 aa, Artificial sequence
  Artificial
KKKKKKKKGGC

SEQ ID NO: 96 - protein, 11 aa, Artificial sequence
  Artificial
KWKKKWKKGCC

SEQ ID NO: 97 - protein, 11 aa, Artificial sequence
  Artificial
RWRRRWRRGGC

SEQ ID NO: 98 - protein, 28 aa, Artificial sequence
  Artificial
GALFLGFLGGAAGSTMGAWSQPKSKRKV

SEQ ID NO: 99 - protein, 26 aa, Artificial sequence
  Artificial
DPKGDPKGVTVTVTVTVTGKGDPKPD

SEQ ID NO: 100 - protein, 18 aa, Artificial sequence
  Artificial
KLALKLALKALKAALKLA

SEQ ID NO: 101 - protein, 18 aa, Artificial sequence
  Artificial
LLIILRRRIRKQAHAHSK

SEQ ID NO: 102 - protein, 16 aa, Artificial sequence
  Artificial
RVIRVWFQNKRCKDKK

SEQ ID NO: 103 - protein, 21 aa, Artificial sequence
  Artificial
KETWWETWWTEWSQPKKKRKV

SEQ ID NO: 104 - protein, 28 aa, Artificial Sequence
  artificial
MANLGYWLLALFVTMWTDVGLCKKRPKP

SEQ ID NO: 105 - protein, 26 aa, Otolemur garnetti
KKYFGKSLALYQKLDLNSVLLKLGIK

SEQ ID NO: 106 - protein, 26 aa, Canis lupus
KKYFGGSLDLYRDLDLNSMLQKLGIK

SEQ ID NO: 107 - protein, 26 aa, Equus caballus
KKYFGKSLDLYKELSLNSMLQKLGIK

SEQ ID NO: 108 - protein, 25 aa, Sus scrofa
KYFGKTLDLYKELALNSTLQKLGIK

SEQ ID NO: 109 - protein, 26 aa, Felis catus
KRYFGGGLDLYQKLALNSMLQKLGIK

SEQ ID NO: 110 - protein, 26 aa, Cricetulus griseus
KKYFGKSLDLYKQLDLNSVLLKFGIK

SEQ ID NO: 111 - protein, 26 aa, Mus musculus
KKYFGKSLDLYKQIDLNSVLQKFGIK

SEQ ID NO: 112 - protein, 25 aa, Rattus norvegicus
KYFGKSLDLYKQIDLNSVLQKFEIK

SEQ ID NO: 113 - protein, 26 aa, Gallus gallus
KKYFSKTLELYQLVNLNGFLLKAGIK

SEQ ID NO: 114 - protein, 26 aa, Crotalus adamanteus
KKYFQKALELYRKIDLNSFLLKVGIK

SEQ ID NO: 115 - protein, 26 aa, Anolis carolinensis
KKYFNKALELYKKLDLNSYLLKLGIK

SEQ ID NO: 116 - protein, 24 aa, Xenopus laevis
KYFSKGLEIYKQVDLNSVLEKSGI

SEQ ID NO: 117 - protein, 21 aa, Danio rerio
KYFGKALELYHKFDLNSVLLK

SEQ ID NO: 118 - protein, 24 aa, Oncorhynchus keta
KYFGKVLELYHMVDLDGVMKKFNI

SEQ ID NO: 119 - protein, 29 aa, Aspergillus niger
EEVQDFFQQVVDLFKTLDSYTALSDAGIT

SEQ ID NO: 120 - protein, 26 aa, Rhizopus niveus
EDIVDYFQKAMDLRSQYNVYKAFSSN

SEQ ID NO: 121 - protein, 25 aa, Escherichia coli
DAYFGTMVRLNGEIKESEAGKFLAD

SEQ ID NO: 122 - protein, 24 aa, Lycopsersicum esculentum
QHAYFKKALDLKNGIDLLSILGGA

SEQ ID NO: 123 - protein, 28 aa, Momordica charantie
QAAYFKLAVDMRNNYSDIIGALRPHAAG

SEQ ID NO: 124 - protein, 28 aa, Nicotiana glutinosa
QHQYFKKALDLKNQINLLEILQQAQINP

SEQ ID NO: 125 - protein, 24 aa, Calystegia sepium
QYEYFSTTLMLYFKYNISEILSES

SEQ ID NO: 126 - protein, 26 aa, Pyrus pyrifolia
ENHYFETVIKMYISKKQNVSRILSKA

SEQ ID NO: 127 - protein, 11 aa, Homo sapiens
YFGRSLELYRE

SEQ ID NO: 128 - protein, 26 aa, Artificial sequence
  the isolated peptide of the invention
  MISC_FEATURE (1)..(2): Each position independently represents  polar, positively charged amino acid residues, His, Arg, Lys or absent
  MISC_FEATURE (1)..(2): present providing residue 3 is present
  MISC_FEATURE (3)..(3): Each position independently represents aromatic residues, Phe, Tyr , Trp or absent
  MISC_FEATURE (4)..(4): Each position independently represents aromatic residues, Phe, Tyr or Trp
  MISC_FEATURE (5)..(5): Represent small, aliphatic, non-polar or slightly polar amino acid residues, Ala, Ser, Thr, Pro orGly
  MISC_FEATURE (6)..(6): polar, positively charged amino acid residues, His, Arg, Lys
  MISC_FEATURE (7)..(7): Represent small, aliphatic, non-polar or slightly polar amino acid residues, Ala, Ser, Thr, Pro orGly
  MISC_FEATURE (8)..(8): large, aliphatic non-polar amino acid residues, Met, Leu, Ile, Val or Cys
  MISC_FEATURE (9)..(9): negatively charged amino acid residues and their (uncharged) amides, Asp, Asn, Glu or Gln
  MISC_FEATURE (10)..(10): large, aliphatic non-polar amino acid residues, Met, Leu, Ile, Val or Cys
  MISC_FEATURE (11)..(11): Each position independently represents aromatic residues, Phe, Tyr or Trp
  MISC_FEATURE (12)..(12): polar, positively charged amino acid residues, His, Arg, Lys
  MISC_FEATURE (13)..(13): negatively charged amino acid residues and their (uncharged) amides, Asp, Asn, Glu or Gln
  MISC_FEATURE (14)..(14): large, aliphatic non-polar amino acid residues, Met, Leu, Ile, Val or Cys
  MISC_FEATURE (15)..(15): negatively charged amino acid residues and their (uncharged) amides, Asp, Asn, Glu or Gln
  MISC_FEATURE (16)..(16): large, aliphatic non-polar amino acid residues, Met, Leu, Ile, Val or Cys
  MISC_FEATURE (17)..(17): negatively charged amino acid residues and their (uncharged) amides, Asp, Asn, Glu or Gln
  MISC_FEATURE (18)..(18): Represent small, aliphatic, non-polar or slightly polar amino acid residues, Ala, Ser, Thr, Pro orGly
  MISC_FEATURE (19)..(21): large, aliphatic non-polar amino acid residues, Met, Leu, Ile, Val or Cys
  MISC_FEATURE (22)..(22): polar, positively charged amino acid residues, His, Arg, Lys
  MISC_FEATURE (23)..(23): large, aliphatic non-polar amino acid residues, Met, Leu, Ile, Val or Cys
  MISC_FEATURE (24)..(24): Represent small, aliphatic, non-polar or slightly polar amino acid residues, Ala, Ser, Thr, Pro orGly
  MISC_FEATURE (25)..(25): large, aliphatic non-polar amino acid residues, Met, Leu, Ile, Val or Cys
  MISC_FEATURE (26)..(26): polar, positively charged amino acid residues, His, Arg, Lys
XXXXXXXXXXXXXXXXXXXXXXXXXX

SEQ ID NO: 129 - protein, 162 aa, homo sapiens
EGCNRSWPFNLEEIKDLLPEMRAYWPDVIHSFPNRSRFWKHEWEKHGTCAAQVDALNSQKKYFGRSLELYRADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 130 - protein, 26 aa, Homo sapiens
  MISC_FEATURE (23)..(23): D-leu
KKYFGRSLELYRELDLNSVLLKLGIK

SEQ ID NO: 131 - protein, 10 aa, Homo sapiens
KKYFGRSLEL

SEQ ID NO: 132 - protein, 5 aa, Homo sapiens
LYREL

SEQ ID NO: 133 - protein, 24 aa, Homo sapiens
KKYFGRSLELYRADFKDALARVYG